(19) United States
(12) Patent Application Publication
(10) Pub. No.: US 2024/0132955 A1
(43) Pub. Date: Apr. 25, 2024

(54) BENIGN SCAR-FORMING CLEAVABLE LINKERS

(71) Applicant: Ultima Genomics, Inc., Newark, CA (US)

(72) Inventors: Linda LEE, Palo Alto, CA (US); Theo Nikiforov, Carlsbad, CA (US); Alexander Joseph Limardo, Pleasanton, CA (US); Maya H. Reamey, Palo Alto, CA (US); Steven Menchen, Fremont, CA (US); Gilad Almogy, Palo Alto, CA (US)

(21) Appl. No.: 18/475,532

(22) Filed: Sep. 27, 2023

Related U.S. Application Data
(63) Continuation of application No. PCT/US2022/022393, filed on Mar. 29, 2022.
(60) Provisional application No. 63/168,048, filed on Mar. 30, 2021.

(51) Int. Cl. C12Q 1/6874; C12Q 1/6806; G01N 21/64

(52) U.S. Cl. CPC C12Q 1/6874; C12Q 1/6806; G01N 21/6486

(57) ABSTRACT

Provided herein are reagents, compositions, and methods for sequencing nucleic acids. Among the reagents disclosed are nucleotides labeled with cleavable linkers. Certain linkers are configured to undergo immolation reactions following cleavage, which can generate scars with enhanced properties for nucleic acid sequencing and synthesis. The present disclosure also provides reagents, compositions, and methods for capping scars generated through linker cleavage, which can alter scar properties, and in some cases can increase the rate and accuracy of nucleotide incorporations during polymerization.

### CROSS-REFERENCE

This application is a continuation of International Patent Application No. PCT/US2022/022393, filed Mar. 29, 2022, which claims benefit of U.S. Provisional Application No. 63/168,048, filed Mar. 30, 2021, which application is entirely incorporated herein by reference.

### STATEMENT AS TO FEDERALLY SPONSORED RESEARCH

This invention was made with government support under Project No. TR44HG011060 awarded by the National Institutes of Health (NIH). The government has certain rights in the invention.

### BACKGROUND

Nucleic acid sequencing is central to a wide range of disciplines. While the speed and accuracy of nucleic acid sequencing methods have improved in recent decades, a number of capabilities lie beyond the scope of modern sequencing methods. In particular, rapid and accurate sequence determination for homopolymeric and repeat regions constitutes a major unmet need within the nucleic acid sequencing space.

### SUMMARY

Sequencing homopolymeric regions with detectably labeled nucleotides presents a wide range of challenges. Sequencing individual nucleotides, for example with reversibly terminated nucleotides, can be prohibitively slow, especially when sequencing large portions of a genome. Conversely, simultaneously detecting multiple adjacent nucleotides can generate quenching interactions between detectable sequencing reagents (e.g., dye-coupled nucleotides). Cleaving detectable labels from reagents can diminish quenching interactions, but can also generate scars which inhibit detection, reagent activity, and nucleic acid polymerization. Therefore, new reagents and methods are needed for sequencing homopolymeric and repeat regions in nucleic acids.

The present disclosure provides reagents, compositions, and methods for efficient nucleic acid sequencing. Numerous aspects of the present disclosure provide detectably labeled nucleotides with low self-quenching efficiencies, and cleavable linkers which generate benign scars. Some labeled nucleotides of the present disclosure comprise self-immolating linkers which spontaneously react following cleavage to generate small, non-inhibitory (e.g., non-polymerase inhibitory) scars. The present disclosure further provides reagents, compositions, and methods for capping nucleotide disposed scars, which can minimize scar inhibitory activity. Accordingly, the reagents, compositions, and methods of the present disclosure can be effective for sequencing long homopolymeric and repeat regions of nucleic acids.

Various aspects of the present disclosure provide a composition comprising a labeled substrate comprising: (a) a nucleotide or an analogue thereof, (b) a detectable moiety; and (c) a linker coupled to said detectable moiety and coupled to said nucleotide or said analogue thereof, wherein said linker comprises a first moiety and a second moiety, and wherein said first moiety is configured to immolate upon or subsequent to cleavage of said second moiety to yield a cleaved linker, a molecular fragment comprising said detectable moiety, and at least two molecular fragments. In some embodiments, said cleaved linker comprises an alcohol or an amine. In some embodiments, said cleaved linker comprises an amine. In some embodiments, said amine is an allyl amine, a vinyl amine, a propargyl amine, an alkynyl amine, an aniline, or a benzyl amine. In some embodiments, said amine is an allyl amine or a propargyl amine. In some embodiments, said amine is a propargyl amine. In some embodiments, said cleaved linker comprises an alcohol. In some embodiments, said alcohol is an allyl alcohol, a vinyl alcohol, a propargyl alcohol, an alkynyl alcohol, a phenol, or a benzyl alcohol. In some embodiments, said alcohol is an allyl alcohol or a propargyl alcohol. said alcohol is a propargyl alcohol. In some embodiments, said cleaved linker terminates in said alcohol or said amine.

In some embodiments, said first moiety is selected from the group consisting of an ester, a thioester, an amide, an imine, a carbonate, a thiocarbonate, a dithiocarbonate, a trithiocarbonate, a carbamate, a thiocarbamate, a dithiocarbamate, a urea, and a thiourea. In some embodiments, said first moiety is selected from the group consisting of a carbonate, a thiocarbonate, a dithiocarbonate, a trithiocarbonate, a carbamate, a thiocarbamate, a dithiocarbamate, a urea, and a thiourea. In some embodiments, said first moiety comprises a carbonate or a carbamate. In some embodiments, said at least two molecular fragments comprises carbon dioxide (CO₂), a thiirane, a cyclohexadienone, a cyclohexadiene thione, or any combination thereof.

In some embodiments, said first moiety is configured to immolate upon or subsequent to cleavage of said second moiety to yield said cleaved linker, said molecular fragment comprising said detectable moiety, and at least three molecular fragments. In some embodiments, said first moiety is configured to immolate upon or subsequent to cleavage of said second moiety to yield said cleaved linker, said molecular fragment comprising said detectable moiety, and at least four molecular fragments. In some embodiments, said first moiety is configured to immolate upon or subsequent to cleavage of said second moiety to yield said cleaved linker, said molecular fragment comprising said detectable moiety, and at least five molecular fragments. In some embodiments, said molecular fragments comprise molecular masses of at most 150 Daltons (Da). In some embodiments, said molecular fragments comprise molecular masses of at most 130 Daltons (Da). In some embodiments, said molecular fragments comprise molecular masses of at most 110 Daltons (Da).

In some embodiments, said cleavage of said second moiety comprises reduction. In some embodiments, said reduction is via one or more reagents selected from the group consisting of tris(3-hydroxypropyl) phosphine (THP), β-mercaptoethanol (β-ME), dithiothreitol (DTT), tris(2-carboxyethyl)phosphine (TCEP), Ellman's reagent, hydroxylamine, glutathione, and cyanoborohydride. In some embodiments, said cleaved linker is coupled to said nucleotide or said analogue thereof.

In some embodiments, said second moiety comprises a chemical group selected from the group consisting of a disulfide group, an azidomethyl group, a hydrocarbyldithiomethyl group, and a 2-nitrobenzyloxy group. In some embodiments, said second moiety comprises a disulfide group. In some embodiments, said disulfide group is selected from the group consisting of a phenylic disulfide, a benzylic disulfide, a pyridyl disulfide, a naphthyl disulfide, a quinolinyl disulfide, a halo disulfide, a nitro disulfide, and an allylic disulfide. In some embodiments, said disulfide group comprises

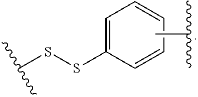

In some embodiments, said linker is coupled to a nucleobase of said nucleotide or said analogue thereof. In some embodiments, said linker is coupled to said nucleobase or said analogue thereof by a propargyl group.

In some embodiments, said linker comprises at least 5 amino acids. In some embodiments, said linker comprises at least 10 amino acids. In some embodiments, said linker comprises at least 15 amino acids. In some embodiments, said linker comprises at least 20 amino acids. In some embodiments, said amino acids comprise a non-proteinogenic amino acid. In some embodiments, said non-proteinogenic amino acid comprises a hydroxyproline. In some embodiments, at least 20%, at least 25%, at least 30, at least 40%, at least 50%, at least 60%, at least 70%, at least 80%, or at least 90% of said amino acids are non-proteinogenic amino acids.

In some embodiments, said linker further comprises a water soluble group. In some embodiments, said water soluble group is coupled to an amino acid of said linker. In some embodiments, said water soluble group is selected from the group consisting of a pyridinium group, an imidazolium group, a quaternary ammonium group, a sulfonate, a phosphate, a hydroxyl, an amine, an imine, a nitrile, an amide, a thiol, a carboxylic acid, a polyether, an aldehyde, a boronic acid, and a boronic ester. In some embodiments, said linker comprises a length of at least about 10, at least about 15, at least about 20, at least about 25, at least about 30, at least about 35, at least about 40, at least about 45, at least about 50, at least about 60, at least about 70, at least about 80, at least about 90, or at least about 100 Angstroms (Å). In some embodiments, said labeling reagent comprises an aqueous solubility of at least 0.01, at least 0.05, at least 0.1, at least 0.5, at least 1, at least 5, at least 10, at least 50, or at least 100 g/L.

In some embodiments, said detectable moiety is selected from the group consisting of a fluorescent dye, a phosphorescent moiety, a luminescent moiety, an electrochemically detectable moiety, and a mass tag. In some embodiments, said detectable moiety comprises a fluorescent dye. In some embodiments, said labeling reagent comprises a half-life of at least 1 day, at least 2 days, at least 3 days, at least 4 days, at least 5 days, at least 7 days, at least 10 days, at least 2 weeks, at least 3 weeks, at least 4 weeks, at least 5 weeks, at least 6 weeks, at least 8 weeks, at least 10 weeks, at least 12 weeks, at least 15 weeks, at least 18 weeks, at least 24 weeks, at least 36 weeks, at least 45 weeks, at least 1 year, at least 1.5 years, at least 2 years, at least 3 years, at least 5 years, or at least 10 years in aqueous solution, in absence of light and reducing agents, and at 25° C.

Various aspects of the present disclosure provide a composition comprising a structure having formula Ia:

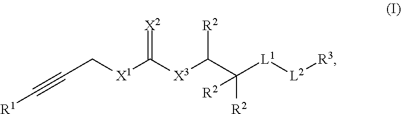

wherein R¹ comprises a substrate selected from the group consisting of a nucleotide, a nucleoside, a nucleobase, an amino acid, a protein, a lipid, a cell, and an antibody; X¹, X², and X³ are independently selected from the group consisting of NR⁴, O, and S; each instance of R² is independently selected from the group consisting of hydrogen, halogen, optionally substituted phenyl, and optionally substituted alkyl; L¹ comprises a cleavable moiety; L² comprises a linker with a length of at least 10 Angstroms (Å); R³ comprises a detectable moiety; and each instance of R⁴ is independently selected from the group consisting of hydrogen, optionally substituted alkyl, and optionally substituted alkoxide.

In some embodiments, said cleavable substrate is configured to form

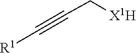

upon cleavage of L¹. In some embodiments said cleavable substrate is configured to form

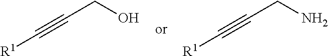

upon cleavage of L¹. In some embodiments, R¹ comprises a nucleotide. In some embodiments, X¹ is NH and X³ is O. In some embodiments, X² is O.

In some embodiments, L¹ comprises a reductively cleavable group, an oxidatively cleavable group, or an enzymatically cleavable group. In some embodiments, L¹ comprises a reductively cleavable group. In some embodiments, said reductively cleavable group comprises a disulfide group. In some embodiments, said disulfide group is selected from the group consisting of a phenylic disulfide, a benzylic disulfide, a pyridyl disulfide, a naphthyl disulfide, a quinolinyl disulfide, a halo disulfide, a nitro disulfide, and an allylic disulfide. In some embodiments, said disulfide group comprises

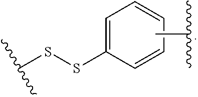

In some embodiments, L² comprises at least 5 amino acids. In some embodiments, L² comprises at least 10 amino acids. In some embodiments, L² comprises at least 20 amino acids. In some embodiments, said amino acids comprise non-proteinogenic amino acids. In some embodiments, said non-proteinogenic amino acids comprise hydroxyproline or pyroglutamic acid. In some embodiments, said non-proteinogenic amino acids are selected from the group consisting of hydroxyproline and pyroglutamic acid. In some embodiments, said at least 5 amino acids comprise a secondary structural feature. In some embodiments, said secondary structural feature comprises a helical structure. In some embodiments, said helical structure comprises a backbone dihedral angle within 20 degrees of planar. In some embodiments, said helical structure comprises a polyproline helix or a polyproline II helix.

In some embodiments, L² comprises a water soluble group. In some embodiments, said water soluble group is selected from the group consisting of a pyridinium group, an imidazolium group, a quaternary ammonium group, a sulfonate, a phosphate, a hydroxyl, an amine, an imine, a nitrile, an amide, a thiol, a carboxylic acid, a polyether, an aldehyde, a boronic acid, and a boronic ester. In some embodiments, said water soluble group is coupled to an amino acid of said at least 5 amino acids.

In some embodiments, L² comprises a length of at least about 15, at least about 20, at least about 25, at least about 30, at least about 35, at least about 40, at least about 45, at least about 50, at least about 60, at least about 70, at least about 80, at least about 90, or at least about 100 Angstroms (Å).

In some embodiments, R³ comprises one or more members selected from the group consisting of an optically detectable moiety, an electrochemically detectable moiety, and a mass tag. In some embodiments, R³ comprises an optically detectable moiety. In some embodiments, said optically detectable moiety comprises a fluorescent dye. In some embodiments, said cleavable substrate comprises an aqueous solubility of at least 0.01, at least 0.05, at least 0.1, at least 0.5, at least 1, at least 5, at least 10, at least 50, or at least 100 g/L.

In some embodiments, said cleavable substrate comprises the structure of formula Ib:

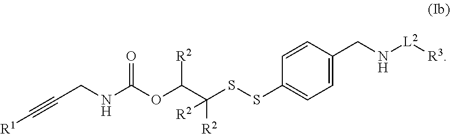

In some embodiments, said cleavable substrate comprises a half-life of at least 1 day, at least 2 days, at least 3 days, at least 4 days, at least 5 days, at least 7 days, at least 10 days, at least 2 weeks, at least 3 weeks, at least 4 weeks, at least 5 weeks, at least 6 weeks, at least 8 weeks, at least 10 weeks, at least 12 weeks, at least 15 weeks, at least 18 weeks, at least 24 weeks, at least 36 weeks, at least 45 weeks, at least 1 year, at least 1.5 years, at least 2 years, at least 3 years, at least 5 years, or at least 10 years in aqueous solution, in absence of light and reducing agents, and at 25° C.

Various aspects of the present disclosure provide a method comprising: contacting a nucleic acid molecule with a first nucleotide, wherein said first nucleotide is coupled to a dye via a cleavable linker, to incorporate said first nucleotide into said nucleic acid molecule; detecting said dye; cleaving said cleavable linker, wherein said cleaving generates a first unlabeled nucleotide comprising a primary amine or a primary hydroxyl moiety; and contacting a second nucleotide to said nucleic acid molecule to incorporate said second nucleotide into said nucleic acid molecule adjacent to said first unlabeled nucleotide, wherein a rate of incorporation of said second nucleotide incorporating adjacent to said first unlabeled nucleotide is at least 2% of a rate of incorporation of said second nucleotide incorporating adjacent to a nucleotide (i) of a same canonical type as said first nucleotide and (ii) that lacks said primary amine or primary hydroxyl moiety.

In some embodiments, said rate of incorporation of said second nucleotide incorporating adjacent to said first unlabeled nucleotide is at least 4%, at least 6%, at least 8%, at least 10%, at least 12%, at least 15%, at least 20%, at least 25%, at least 30%, at least 40%, or at least 50% of a rate of incorporation of said second nucleotide incorporating adjacent to a nucleotide (i) of a same canonical type as said first nucleotide and (ii) that lacks said primary amine or primary hydroxyl moiety. In some embodiments, said primary amine or primary hydroxyl moiety comprises a propargyl amine or propargyl alcohol.

In some embodiments, said cleaving comprises contacting said first nucleotide with a reducing agent. In some embodiments, said reducing reagent comprises one or more members selected from the group consisting of: tetrahydropyran, β-mercaptoethanol (β-ME), dithiothreitol (DTT), tris(2-carboxyethyl)phosphine (TCEP), Ellman's reagent, hydroxylamine, and cyanoborohydride. In some embodiments, said cleaving uncouples said dye from said first nucleotide.

In some embodiments, said contacting said nucleic acid molecule with said first nucleotide comprises contacting said nucleic acid molecule with a mixture comprising a first population of nucleotides coupled to dyes via cleavable linkers and a second population of nucleotides not coupled to dyes via cleavable linkers. In some embodiments, said mixture comprises a single canonical type of nucleotide. In some embodiments, said contacting said nucleic acid molecule with said first nucleotide comprises contacting said nucleic acid molecule with a single population of nucleotides, wherein about 100% of nucleotides from said single population of nucleotides are coupled to dyes via cleavable linkers.

In some embodiments, said cleavable linker comprises a first moiety and a second moiety, and wherein said cleaving comprises cleavage of said second moiety, and wherein said first moiety is configured to immolate upon or subsequent to said cleavage of said second moiety. In some embodiments, said first moiety is selected from the group consisting of an ester, a thioester, an amide, an imine, a carbonate, a thiocarbonate, a dithiocarbonate, a trithiocarbonate, a carbamate, a thiocarbamate, a dithiocarbamate, a urea, and a thiourea. In some embodiments, said first moiety comprises a carbonate or a carbamate. In some embodiments, immolation of said first moiety generates carbon dioxide (CO₂), a thiirane, or any combination thereof. In some embodiments, said second moiety comprises a disulfide group, an azidomethyl group, a hydrocarbyldithiomethyl group, a 2-nitrobenyloxy group, or any combination thereof. In some embodiments, said second moiety comprises a disulfide group. In some embodiments, said disulfide group comprises

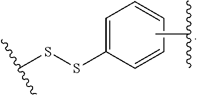

In some embodiments, said incorporation of said first nucleotide into said nucleic acid molecule comprises a rate constant of at least 0.01 M⁻¹s⁻¹, at least 0.1 M⁻¹s⁻¹, or at least 1 M⁻¹s⁻¹ at 25° C. In some embodiments, said conditions suitable for hybridization are multiple turnover conditions. In some embodiments, said rate of incorporation of said second nucleotide incorporating adjacent to said first unlabeled nucleotide is increased by at least 5-fold, 10-fold, at least 50-fold, 100-fold, at least 500-fold, at least 1000-fold, or at least 5000-fold upon said cleaving of said cleavable linker of said first nucleotide. In some embodiments, said second nucleotide is coupled to a dye via a cleavable linker, and wherein said dye coupled to said first nucleotide is different than said dye coupled to said second nucleotide.

In some embodiments, said contacting said nucleic acid molecule with said second nucleotide comprises contacting said nucleic acid molecule with a second mixture comprising a third population of nucleotides coupled to dyes via cleavable linkers and a fourth population of nucleotides not coupled to dyes via cleavable linkers. In some embodiments, said second mixture comprises a single canonical type of nucleotide. In some embodiments, said contacting said nucleic acid molecule with said second nucleotide comprises contacting said nucleic acid molecule with a single population of nucleotides, wherein about 100% of nucleotides from said single population of nucleotides are coupled to dyes via cleavable linkers.

In some embodiments, said nucleic acid molecule is immobilized to a support. In some embodiments, said support comprises a bead, sphere, particle, granule, gel, porous matrix, surface, or any combination thereof. In some embodiments, said nucleic acid molecule is immobilized to said support by streptavidin. In some embodiments, said detecting comprises imaging.

In some embodiments, said incorporation of said second nucleotide comprises a misincorporation rate of less than 10%. In some embodiments, said incorporation of said second nucleotide comprises a misincorporation rate of less than 5%. In some embodiments, said incorporation of said second nucleotide comprises a misincorporation rate of less than 1%.

Various aspects of the present disclosure provide a method for sequencing, comprising: (i) incorporating a nucleotide into a growing nucleic acid strand hybridized to a nucleic acid molecule, wherein said nucleotide is coupled to a dye via a cleavable linker; (ii) detecting a signal or change thereof from said dye; (iii) cleaving said cleavable linker to generate a reactive moiety on said nucleotide; (iv) contacting a capping reagent to said reactive moiety to generate a capped moiety; and (v) incorporating an additional nucleotide adjacent to said nucleotide in said growing nucleic acid strand. In some embodiments, said method further comprises repeating (i)-(v). In some embodiments, said nucleotide is a terminated nucleotide. In some embodiments, said nucleotide is a non-terminated nucleotide.

In some embodiments, (i) comprises contacting said growing nucleic acid strand with a reaction mixture comprising a plurality of nucleotides of a same canonical base type. In some embodiments, said contacting to incorporates at least two nucleotides into said growing nucleic acid strand, wherein said at least two nucleotides are coupled to dyes via cleavable linkers, and wherein said at least two nucleotides comprises said nucleotide. In some embodiments, said plurality of nucleotides comprises a subset of labeled nucleotides and a subset of unlabeled nucleotides.

In some embodiments, said cleavable linker comprises a length of at least at least about 10, about 15, about 20, about 25, about 30, about 35, about 40, about 45, about 50, about 60, about 70, about 80, about 90, or about 100 Angstroms (Å). In some embodiments, said cleaving comprises splitting a disulfide of said cleavable linker. In some embodiments, said cleaving comprises reduction of said cleavable linker. In some embodiments, said reduction is via one or more reducing agents selected from the group consisting of tetrahydropyran, β-mercaptoethanol (β-ME), dithiothreitol (DTT), tris(2-carboxyethyl)phosphine (TCEP), Ellman's reagent, hydroxylamine, glutathione, and cyanoborohydride. In some embodiments, said cleaving comprises a rate constant between 0.05 and 5, 0.1 and 10, or 1 and 100 mM⁻¹min⁻¹ at 25° C. In some embodiments, said reactive moiety comprises a thiol. In some embodiments, said capping reagent comprises a reversible capping reagent.

In some embodiments, said cleaving comprises removing an additional reversible capping reagent from said growing nucleic acid strand. In some embodiments, said reversible capping reagent is selected from the group consisting of dipyridyl disulfide (DPDS) comprises 4-4′-dipyridyl disulfide, 2,2′-dithiobis(5-nitropyridine), 6,6′-dithiodinicotinic acid, and dipyridyl disulfide. In some embodiments, said capping reagent comprises an irreversible capping reagent. In some embodiments, said irreversible capping reagent is selected from the group consisting of a haloacetamide, a maleimide, and a propioloate. In some embodiments, (v) comprises a rate constant of at least 0.05, at least 0.1, at least 0.5, at least 1, at least 5, at least 10, or at least 50 mM⁻¹min⁻¹ at 25° C. In some embodiments, said capping reagent is inert toward nucleoside moieties of said growing nucleic acid strand and said nucleic acid molecule.

Various aspects of the present disclosure provide a method for sequencing, comprising: (a) incorporating a nucleotide into a growing nucleic acid strand hybridized to a nucleic acid molecule, wherein said nucleotide is coupled to a dye via a cleavable linker; (b) detecting a signal or change thereof from said dye; (c) cleaving said cleavable linker to generate a reactive moiety on said nucleotide; (d) contacting said growing nucleic acid strand with a mixture comprising a capping reagent and an additional nucleotide, wherein said capping reagent is configured to couple to said reactive moiety to generate a capped moiety.

In some embodiments, said mixture comprises a plurality of nucleotides of a same canonical nucleotide type. In some embodiments, at least 75%, at least 80%, at least 85%, at least 90%, at least 95%, at least 98%, or at least 99% nucleotides of said plurality of nucleotides are of a single canonical nucleotide type. In some embodiments, said plurality of nucleotides comprises a subset of labeled nucleotides and a subset of unlabeled nucleotides. In some embodiments, said mixture comprises a plurality of nucleotides, wherein at least 75%, at least 80%, at least 85%, at least 90%, at least 95%, at least 98%, or at least 99% nucleotides of said plurality of nucleotides are of two canonical nucleotide types, and wherein the two canonical nucleotide types are coupled to different detectable moieties. In some embodiments, said different detectable moieties comprise different fluorescent dyes. In some embodiments, said plurality of nucleotides comprises said additional nucleotide. In some embodiments, said mixture comprises a plurality of nucleotides of a same canonical base type, wherein said mixture does not comprise a labeled nucleotide, wherein said plurality of nucleotides comprises said additional nucleotide. In some embodiments, said nucleotide and said additional nucleotide comprise a same type of canonical nucleobase.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 provides a table summarizing relative nucleotide incorporation rates at positions adjacent to multiple types of scarred nucleotides.

FIG. 2 provides a reaction scheme for an example of linker cleavage with a post-cleavage reaction.

FIG. 3 provides theoretical fluorescence intensities as a function of the number of fluorophores coupled to a single molecule (e.g., a nucleic acid molecule) at multiple quenching efficiencies.

FIG. 4 illustrates a method for nucleic acid sequencing by extending a primer with labeled nucleotides.

FIG. 5 provides chemical reaction schemes for exemplary thiol capping methods. Panel A illustrates a reversible thiol capping mechanism with the capping reagent dipyridine disulfide. Panel B illustrates a reversible thiol capping mechanism with a thiosulfate capping reagent. Panel C illustrates a reversible thiol capping mechanism with an alkyne capping reagent. Panel D illustrates an irreversible thiol capping mechanism with the capping reagent iodoacetamide. Panel E illustrates an irreversible thiol capping mechanism with an N-ethylmaleimide capping reagent.

FIG. 6 illustrates a synthesis method for a labeled nucleotide with a pyridine thiolate cap.

FIG. 7 provides a chemical scheme for linker cleavage and immolation for two labeled uridine triphosphate substrates.

FIG. 8 provides mass spectra of the cleavage and immolation products of the two labeled nucleotides presented in FIG. 7. FIGS. 8A and 8B provide the cleavage and immolation products of the two labeled nucleotides immediately following cleavage. FIG. 8C provides the cleavage and immolation products of the slower-immolating nucleotide 10 minutes after cleavage. FIG. 8D provides the cleavage and immolation products of the slower-immolating nucleotide 30 minutes after cleavage.

FIGS. 9A-9E provide structures and homopolymerization results for two types of labeled nucleotides. FIGS. 9A and 9B provide structures for two types of labeled nucleotides, differing primarily in linker length. FIG. 9C provides a fluorescence image of variable length, gel electrophoresis-separated products of homopolymerization reactions performed with the labeled nucleotide of FIG. 9A. FIG. 9D provides a fluorescence image of variable length, gel electrophoresis-separated products of homopolymerization reactions performed with the labeled nucleotide of FIG. 9B. FIG. 9E summarizes the fluorescence intensities of the various length homopolymer products imaged in FIGS. 9C and 9D.

FIGS. 10A-10E provide structures and nucleic acid extension results for two different types of labeled nucleotides. FIG. 10A provides the structures of the labeled adenosine, cytidine, and guanosine nucleotides. FIG. 10B provides the structures of the labeled uridine nucleotides. FIG. 10C summarizes the sequencing results of an assay utilizing flows with 100% labeled U, 10% labeled A, 20% labeled C, 10% labeled G, and no capping reagents. FIG. 10D summarizes the sequencing results of an assay utilizing flows with 100% labeled U, 10% labeled A, 100% labeled C, 10% labeled G, and no capping reagents. FIG. 10E summarizes the sequencing results of an assay utilizing flows with 100% labeled U, 10% labeled A, 20% labeled C, 10% labeled G, and a capping reagent provided in between labeled U and labeled A flows.

FIG. 11 provides results from sequencing assays utilizing different capping reagents.

FIG. 12A provides a structure for a labeled guanosine nucleotide. FIG. 12B provides a chemical scheme for cleavage and capping of the labeled guanosine nucleotide from FIG. 12A.

FIG. 13 provides fluorescence results from nucleic acid homopolymer extension assays utilizing the labeled guanosine nucleotide of FIG. 12A and the cleaved and capped guanosine nucleotide 1202 of FIG. 12B.

FIGS. 14A and 14B provide cleavage and immolation reaction mechanisms for two types of labeled nucleotides. The labeled nucleotides in the embodiments provided in FIGS. 14A and 14B comprise deoxyribonucleotides.

FIG. 15A illustrates a scheme for a nucleotide extension assay utilizing the labeled nucleotides of FIGS. 14A and 14B. FIG. 15B provides fluorescence time course results of the assay outlined in FIG. 15A utilizing the labeled nucleotides of FIG. 14A. FIG. 15C provides fluorescence time course results of the assay outlined in FIG. 15A utilizing the labeled nucleotides of FIG. 14B.

FIG. 16A summarizes fluorescence results from a sequencing assay utilizing flows with 100% labeled U nucleotides, 10% labeled A nucleotides, 20% labeled C nucleotides, 10% labeled G nucleotides, no capping reagent, and non-immolating linkers.

FIG. 16B summarizes fluorescence results from a sequencing assay utilizing flows with 100% labeled U nucleotides, 10% labeled A nucleotides, 20% labeled C nucleotides, 10% labeled G nucleotides, along with a capping reagent provided in separate 100% unlabeled U flows.

FIG. 16C summarizes fluorescence results from a sequencing assay utilizing flows with 100% labeled U nucleotides, 10% labeled A nucleotides, 20% labeled C nucleotides, 10% labeled G nucleotides, no capping reagent, and immolating linkers in the labeled U nucleotides.

FIGS. 17A and 17B provide different labeled nucleotides with cleavable linkers. FIG. 17A provides a labeled nucleotide with a cleavable linker that is not configured for an immolation reaction. FIG. 17B provides a labeled nucleotide with a cleavable linker configured to undergo an immolation reaction following cleavage.

FIGS. 17C and 17D provide fluorescence results from nucleic acid sequencing assays utilizing the labeled nucleotides from FIGS. 17A and 17B, respectively.

FIG. 18 provides a chemical scheme for cleavage and immolation reactions for a linker in which the cleavable group is spaced from a portion of the immolating group by an aromatic moiety.

FIG. 19 shows an example of a method for preparing a labeled nucleotide comprising a dGTP analog.

FIG. 20 provides a scheme for modular labeled nucleotide synthesis.

FIGS. 21A and 21B show exemplary rates of homopolymer detection (e.g., the linearity between recorded signal and homopolymer length) in sequencing assays performed with and without DPDS as a capping agent, respectively.

FIGS. 22A and 22B illustrate labeled nucleotides with variable length linker regions disposed between propargyl amine functionalized guanosine nucleotide substrates and oligonucleotide linker moieties.

FIG. 23 provides extension results for the substrate of FIG. 22A over variable length homopolymeric deoxycytidine phosphate regions. Panel A provides corrected fluorescence intensities of the oligodeoxyguanosine phosphate-containing extension products. Panel B provides the data from Panel A plotted as corrected fluorescence intensities as a function of oligocytidine template length. Panel C provides classification errors for homopolymer length identifications based on the fluorescence data of Panels A and B.

FIG. 24 provides extension results for the substrate of FIG. 22B over variable length homopolymeric deoxycytidine phosphate regions. Panel A provides corrected fluorescence intensities of the oligodeoxyguanosine phosphate-containing extension products. Panel B provides the data from Panel A plotted as corrected fluorescence intensities as a function of oligodeoxycytidine phosphate template length. Panel C provides classification errors for homopolymer length identifications based on the fluorescence data of Panels A and B.

FIG. 25 provides extension results for a labeled cytidine triphosphate substrate over variable length homopolymeric regions. Panel A provides the structure of the labeled nucleotide substrate. Panel B provides corrected fluorescence intensities of the oligocytidine-containing extension products. Panel C provides the data from Panel B plotted as corrected fluorescence intensities as a function of oligoguanidine template length. Panel D provides classification errors for homopolymer length identifications based on the fluorescence data of Panels B and C.

FIG. 26 provides extension results for a labeled adenosine triphosphate substrate over variable length homopolymeric regions. Panel A provides the structure of the labeled nucleotide substrate. Panel B provides corrected fluorescence intensities of the oligoadenosine-containing extension products. Panel C provides the data from Panel B plotted as corrected fluorescence intensities as a function of template length. Panel D provides classification errors for homopolymer length identifications based on the fluorescence data of Panels B and C.

FIG. 27 provides extension results for a labeled uridine triphosphate substrate over variable length homopolymeric regions. Panel A provides the structure of the labeled nucleotide substrate. Panel B provides corrected fluorescence intensities of the oligouridine-containing extension products. Panel C provides the data from Panel B plotted as corrected fluorescence intensities as a function of template length. Panel D provides classification errors for homopolymer length identifications based on the fluorescence data of Panels B and C.

FIG. 28 provides extension results for a labeled guanosine triphosphate substrate over variable length homopolymeric regions. Panel A provides the structure of the labeled nucleotide substrate. Panel B provides corrected fluorescence intensities of the oligoguanosine-containing extension products. Panel C provides the data from Panel B plotted as corrected fluorescence intensities as a function of template length. Panel D provides classification errors for homopolymer length identifications based on the fluorescence data of Panels B and C.

FIG. 29 panels A and B provide cleavage and post-cleavage reaction schemes for two nucleotide substrates labeled with different cleavable linkers.

FIG. 30 provides cleavage and post-cleavage reaction mechanisms for a labeled dUTP product.

FIGS. 31A and 31B provide structures for labeled nucleotide substrates.

FIG. 32 shows a computer system that is programmed or otherwise configured to implement methods provided herein.

FIGS. 33A and 33B provide the chemical structures for the U*-PHH (FIG. 33A) and U*-QHH (FIG. 33B).

FIG. 34 provides extension results for the substrate U*-YHH/DPDS over variable length homopolymeric deoxycytidine phosphate regions. The U*-YHH linker leaves a thiol that is subsequently capped with DPDS. Panel A provides corrected fluorescence intensities of the oligodeoxyguanosine phosphate-containing extension products. Panel B provides the data from Panel A plotted as corrected fluorescence intensities as a function of oligocytidine template length. Panel C provides classification errors for homopolymer length identifications based on the fluorescence data of Panels A and B.

FIG. 35 provides extension results for the substrate of U*-PHH over variable length homopolymeric deoxycytidine phosphate regions. Panel A provides corrected fluorescence intensities of the oligodeoxyguanosine phosphate-containing extension products. Panel B provides the data from Panel A plotted as corrected fluorescence intensities as a function of oligocytidine template length. Panel C provides classification errors for homopolymer length identifications based on the fluorescence data of Panels A and B.

FIG. 36 provides extension results for the substrate of U*-QHH over variable length homopolymeric deoxycytidine phosphate regions. Panel A provides corrected fluorescence intensities of the oligodeoxyguanosine phosphate-containing extension products. Panel B provides the data from Panel A plotted as corrected fluorescence intensities as a function of oligocytidine template length. Panel C provides classification errors for homopolymer length identifications based on the fluorescence data of Panels A and B.

FIG. 37 shows sequencing data for labeled uracil-containing nucleotides including different cleavable moieties.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The terms “about” and “approximately” shall generally mean an acceptable degree of error or variation for a given value or range of values, such as, for example, a degree of error or variation that is within 20 percent (%), within 15%, within 10%, or within 5% of a given value or range of values.

The term “subject,” as used herein, generally refers to an individual or entity from which a biological sample (e.g., a biological sample that is undergoing or will undergo processing or analysis) may be derived. A subject may be an animal (e.g., mammal or non-mammal) or plant. The subject may be a human, dog, cat, horse, pig, bird, non-human primate, simian, farm animal, companion animal, sport animal, or rodent. A subject may be a patient. The subject may have or be suspected of having a disease or disorder, such as cancer (e.g., breast cancer, colorectal cancer, brain cancer, leukemia, lung cancer, skin cancer, liver cancer, pancreatic cancer, lymphoma, esophageal cancer, or cervical cancer) or an infectious disease. Alternatively or in addition to, a subject may be known to have previously had a disease or disorder. The subject may have or be suspected of having a genetic disorder such as achondroplasia, alpha-1 antitrypsin deficiency, antiphospholipid syndrome, autism, autosomal dominant polycystic kidney disease, Charcot-Marie-tooth, cri du chat, Crohn's disease, cystic fibrosis, Dercum disease, down syndrome, Duane syndrome, Duchenne muscular dystrophy, factor V Leiden thrombophilia, familial hypercholesterolemia, familial Mediterranean fever, fragile x syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency, sickle cell disease, spinal muscular atrophy, Tay-Sachs, thalassemia, trimethylaminuria, Turner syndrome, velocardiofacial syndrome, WAGR syndrome, or Wilson disease. A subject may be undergoing treatment for a disease or disorder. A subject may be symptomatic or asymptomatic of a given disease or disorder. A subject may be healthy (e.g., not suspected of having disease or disorder). A subject may have one or more risk factors for a given disease. A subject may have a given weight, height, body mass index, or other physical characteristic. A subject may have a given ethnic or racial heritage, place of birth or residence, nationality, disease or remission state, family medical history, or other characteristic.

As used herein, the term “biological sample” generally refers to a sample obtained from a subject. The biological sample may be obtained directly or indirectly from the subject. A sample may be obtained from a subject via any suitable method, including, but not limited to, spitting, swabbing, blood draw, biopsy, obtaining excretions (e.g., urine, stool, sputum, vomit, or saliva), excision, scraping, and puncture. A sample may be obtained from a subject by, for example, intravenously or intraarterially accessing the circulatory system, collecting a secreted biological sample (e.g., stool, urine, saliva, sputum, etc.), breathing, or surgically extracting a tissue (e.g., biopsy). The sample may be obtained by non-invasive methods including but not limited to: scraping of the skin or cervix, swabbing of the cheek, or collection of saliva, urine, feces, menses, tears, or semen. Alternatively, the sample may be obtained by an invasive procedure such as biopsy, needle aspiration, or phlebotomy. A sample may comprise a bodily fluid such as, but not limited to, blood (e.g., whole blood, red blood cells, leukocytes or white blood cells, platelets), plasma, serum, sweat, tears, saliva, sputum, urine, semen, mucus, synovial fluid, breast milk, colostrum, amniotic fluid, bile, bone marrow, interstitial or extracellular fluid, or cerebrospinal fluid. For example, a sample may be obtained by a puncture method to obtain a bodily fluid comprising blood and/or plasma. Such a sample may comprise both cells and cell-free nucleic acid material. Alternatively, the sample may be obtained from any other source including but not limited to blood, sweat, hair follicle, buccal tissue, tears, menses, feces, or saliva. The biological sample may be a tissue sample, such as a tumor biopsy. The sample may be obtained from any of the tissues provided herein including, but not limited to, skin, heart, lung, kidney, breast, pancreas, liver, intestine, brain, prostate, esophagus, muscle, smooth muscle, bladder, gall bladder, colon, or thyroid. The methods of obtaining provided herein include methods of biopsy including fine needle aspiration, core needle biopsy, vacuum assisted biopsy, large core biopsy, incisional biopsy, excisional biopsy, punch biopsy, shave biopsy or skin biopsy. The biological sample may comprise one or more cells. A biological sample may comprise one or more nucleic acid molecules such as one or more deoxyribonucleic acid (DNA) and/or ribonucleic acid (RNA) molecules (e.g., included within cells or not included within cells). Nucleic acid molecules may be included within cells. Alternatively or in addition to, nucleic acid molecules may not be included within cells (e.g., cell-free nucleic acid molecules). The biological sample may be a cell-free sample.

The term “cell-free sample,” as used herein, generally refers to a sample that is substantially free of cells (e.g., less than 10% cells on a volume basis). A cell-free sample may be derived from any source (e.g., as described herein). For example, a cell-free sample may be derived from blood, sweat, urine, or saliva. For example, a cell-free sample may be derived from a tissue or bodily fluid. A cell-free sample may be derived from a plurality of tissues or bodily fluids. For example, a sample from a first tissue or fluid may be combined with a sample from a second tissue or fluid (e.g., while the samples are obtained or after the samples are obtained). In an example, a first fluid and a second fluid may be collected from a subject (e.g., at the same or different times) and the first and second fluids may be combined to provide a sample. A cell-free sample may comprise one or more nucleic acid molecules such as one or more DNA or RNA molecules.

A sample that is not a cell-free sample (e.g., a sample comprising one or more cells) may be processed to provide a cell-free sample. For example, a sample that includes one or more cells as well as one or more nucleic acid molecules (e.g., DNA and/or RNA molecules) not included within cells (e.g., cell-free nucleic acid molecules) may be obtained from a subject. The sample may be subjected to processing (e.g., as described herein) to separate cells and other materials from the nucleic acid molecules not included within cells, thereby providing a cell-free sample (e.g., comprising nucleic acid molecules not included within cells). The cell-free sample may then be subjected to further analysis and processing (e.g., as provided herein). Nucleic acid molecules not included within cells (e.g., cell-free nucleic acid molecules) may be derived from cells and tissues. For example, cell-free nucleic acid molecules may derive from a tumor tissue or a degraded cell (e.g., of a tissue of a body). Cell-free nucleic acid molecules may comprise any type of nucleic acid molecules (e.g., as described herein). Cell-free nucleic acid molecules may be double-stranded, single-stranded, or a combination thereof. Cell-free nucleic acid molecules may be released into a bodily fluid through secretion or cell death processes, e.g., cellular necrosis, apoptosis, or the like. Cell-free nucleic acid molecules may be released into bodily fluids from cancer cells (e.g., circulating tumor DNA (ctDNA)). Cell free nucleic acid molecules may also be fetal DNA circulating freely in a maternal blood stream (e.g., cell-free fetal nucleic acid molecules such as cffDNA). Alternatively or in addition to, cell-free nucleic acid molecules may be released into bodily fluids from healthy cells.

A biological sample may be obtained directly from a subject and analyzed without any intervening processing, such as, for example, sample purification or extraction. For example, a blood sample may be obtained directly from a subject by accessing the subject's circulatory system, removing the blood from the subject (e.g., via a needle), and transferring the removed blood into a receptacle. The receptacle may comprise reagents (e.g., anti-coagulants) such that the blood sample is useful for further analysis. Such reagents may be used to process the sample or analytes derived from the sample in the receptacle or another receptacle prior to analysis. In another example, a swab may be used to access epithelial cells on an oropharyngeal surface of the subject. Following obtaining the biological sample from the subject, the swab containing the biological sample may be contacted with a fluid (e.g., a buffer) to collect the biological fluid from the swab.

Any suitable biological sample that comprises one or more nucleic acid molecules may be obtained from a subject. A sample (e.g., a biological sample or cell-free biological sample) suitable for use according to the methods provided herein may be any material comprising tissues, cells, degraded cells, nucleic acids, genes, gene fragments, expression products, gene expression products, and/or gene expression product fragments of an individual to be tested. A biological sample may be solid matter (e.g., biological tissue) or may be a fluid (e.g., a biological fluid). In general, a biological fluid may include any fluid associated with living organisms. Non-limiting examples of a biological sample include blood (or components of blood—e.g., white blood cells, red blood cells, platelets) obtained from any anatomical location (e.g., tissue, circulatory system, bone marrow) of a subject, cells obtained from any anatomical location of a subject, skin, heart, lung, kidney, breath, bone marrow, stool, semen, vaginal fluid, interstitial fluids derived from tumorous tissue, breast, pancreas, cerebral spinal fluid, tissue, throat swab, biopsy, placental fluid, amniotic fluid, liver, muscle, smooth muscle, bladder, gall bladder, colon, intestine, brain, cavity fluids, sputum, pus, microbiota, meconium, breast milk, prostate, esophagus, thyroid, serum, saliva, urine, gastric and digestive fluid, tears, ocular fluids, sweat, mucus, earwax, oil, glandular secretions, spinal fluid, hair, fingernails, skin cells, plasma, nasal swab or nasopharyngeal wash, spinal fluid, cord blood, emphatic fluids, and/or other excretions or body tissues. Methods for determining sample suitability and/or adequacy are provided. A sample may include, but is not limited to, blood, plasma, tissue, cells, degraded cells, cell-free nucleic acid molecules, and/or biological material from cells or derived from cells of an individual such as cell-free nucleic acid molecules. The sample may be a heterogeneous or homogeneous population of cells, tissues, or cell-free biological material. The biological sample may be obtained using any method that can provide a sample suitable for the analytical methods described herein.

A sample (e.g., a biological sample or cell-free biological sample) may undergo one or more processes in preparation for analysis, including, but not limited to, filtration, centrifugation, selective precipitation, permeabilization, isolation, agitation, heating, purification, and/or other processes. For example, a sample may be filtered to remove contaminants or other materials. In an example, a sample comprising cells may be processed to separate the cells from other material in the sample. Such a process may be used to prepare a sample comprising only cell-free nucleic acid molecules. Such a process may consist of a multi-step centrifugation process. Multiple samples, such as multiple samples from the same subject (e.g., obtained in the same or different manners from the same or different bodily locations, and/or obtained at the same or different times (e.g., seconds, minutes, hours, days, weeks, months, or years apart)) or multiple samples from different subjects may be obtained for analysis as described herein. In an example, the first sample is obtained from a subject before the subject undergoes a treatment regimen or procedure and the second sample is obtained from the subject after the subject undergoes the treatment regimen or procedure. Alternatively or additionally, multiple samples may be obtained from the same subject at the same or approximately the same time. Different samples obtained from the same subject may be obtained in the same or different manner. For example, a first sample may be obtained via a biopsy and a second sample may be obtained via a blood draw. Samples obtained in different manners may be obtained by different medical professionals, using different techniques, at different times, and/or at different locations. Different samples obtained from the same subject may be obtained from different areas of a body. For example, a first sample may be obtained from a first area of a body (e.g., a first tissue) and a second sample may be obtained from a second area of the body (e.g., a second tissue).

A biological sample as used herein (e.g., a biological sample comprising one or more nucleic acid molecules) may not be purified when provided in a reaction vessel. Furthermore, for a biological sample comprising one or more nucleic acid molecules, the one or more nucleic acid molecules may not be extracted when the biological sample is provided to a reaction vessel. For example, ribonucleic acid (RNA) and/or deoxyribonucleic acid (DNA) molecules of a biological sample may not be extracted from the biological sample when providing the biological sample to a reaction vessel. Moreover, a target nucleic acid (e.g., a target RNA or target DNA molecules) present in a biological sample may not be concentrated when providing the biological sample to a reaction vessel. Alternatively, a biological sample may be purified and/or nucleic acid molecules may be isolated from other materials in the biological sample.

A biological sample as described herein may contain a target nucleic acid. As used herein, the terms “template nucleic acid,” “target nucleic acid,” “nucleic acid molecule,” “nucleic acid sequence,” “nucleic acid fragment,” “oligonucleotide,” “polynucleotide,” and “nucleic acid” generally refer to polymeric forms of nucleotides of any length, such as deoxyribonucleotides (dNTPs) or ribonucleotides (rNTPs), or analogs thereof, and may be used interchangeably. Nucleic acids may have any three-dimensional structure, and may perform any function, known or unknown. A nucleic acid molecule may have a length of at least about 10 nucleic acid bases (“bases”), 20 bases, 30 bases, 40 bases, 50 bases, 100 bases, 200 bases, 300 bases, 400 bases, 500 bases, 1 kilobase (kb), 2 kb, 3, kb, 4 kb, 5 kb, 10 kb, 50 kb, or more. An oligonucleotide is typically composed of a specific sequence of four nucleotide bases: adenine (A); cytosine (C); guanine (G); and thymine (T) (uracil (U) for thymine (T) when the polynucleotide is RNA). Oligonucleotides may include one or more nonstandard nucleotide(s), nucleotide analog(s) and/or modified nucleotides. Non-limiting examples of nucleic acids include DNA, RNA, genomic DNA (e.g., gDNA such as sheared gDNA), cell-free DNA (e.g., cfDNA), synthetic DNA/RNA, coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, complementary DNA (cDNA), recombinant nucleic acids, branched nucleic acids, plasmids, vectors, isolated DNA of any sequence, isolated RNA of any sequence, nucleic acid probes, and primers. A nucleic acid may comprise one or more modified nucleotides, such as methylated nucleotides and nucleotide analogs. If present, modifications to the nucleotide structure may be made before or following assembly of the nucleic acid. The sequence of nucleotides of a nucleic acid may be interrupted by non-nucleotide components. A nucleic acid may be further modified following polymerization, such as by conjugation or binding with a reporter agent.

A target nucleic acid or sample nucleic acid as described herein may be amplified to generate an amplified product. A target nucleic acid may be a target RNA or a target DNA. When the target nucleic acid is a target RNA, the target RNA may be any type of RNA, including types of RNA described elsewhere herein. The target RNA may be viral RNA and/or tumor RNA. A viral RNA may be pathogenic to a subject. Non-limiting examples of pathogenic viral RNA include human immunodeficiency virus I (HIV I), human immunodeficiency virus n (HIV 11), orthomyxoviruses, Ebola virus. Dengue virus, influenza viruses (e.g., H1N1, H3N2, H7N9, or H5N1), herpesvirus, hepatitis A virus, hepatitis B virus, hepatitis C (e.g., armored RNA-HCV virus) virus, hepatitis D virus, hepatitis E virus, hepatitis G virus, Epstein-Barr virus, mononucleosis virus, cytomegalovirus, SARS virus, West Nile Fever virus, polio virus, and measles virus.

A biological sample may comprise a plurality of target nucleic acid molecules. For example, a biological sample may comprise a plurality of target nucleic acid molecules from a single subject. In another example, a biological sample may comprise a first target nucleic acid molecule from a first subject and a second target nucleic acid molecule from a second subject.

The term “nucleotide,” as used herein, generally refers to a substance including a base (e.g., a nucleobase), sugar moiety, and phosphate moiety. A nucleotide may comprise a free base with attached phosphate groups. A nucleotide may comprise a nucleoside monophosphate, a nucleoside diphosphate, or a nucleoside triphosphate. When a nucleotide is being added to a growing nucleic acid molecule strand, the formation of a phosphodiester bond between the proximal phosphate of the nucleotide and the growing chain may be accompanied by hydrolysis of a high-energy phosphoanhydride bond with release of the two distal phosphates as a pyrophosphate. The nucleotide may be naturally occurring or non-naturally occurring (e.g., a modified or engineered nucleotide).

The term “nucleotide analog,” as used herein, may include, but is not limited to, a nucleotide that may or may not be a naturally occurring nucleotide. For example, a nucleotide analog may be derived from and/or include structural similarities to a canonical nucleotide such as adenosine—(A), thymidine—(T), cytidine—(C), uridine—(U), or guanosine—(G) including nucleotide. A nucleotide analog may comprise one or more differences or modifications relative to a natural nucleotide. Examples of nucleotide analogs include inosine, diaminopurine, 5-fluorouracil, 5-bromouracil, 5-chlorouracil, 5-iodouracil, hypoxanthine, xanthine, deazaxanthine, deazaguanine, isocytosine, isoguanine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine, ethynyl nucleotide bases, 1-propynyl nucleotide bases, azido nucleotide bases, phosphoroselenoate nucleic acids, and modified versions thereof (e.g., by oxidation, reduction, and/or addition of a substituent such as an alkyl, hydroxyalkyl, hydroxyl, or halogen moiety). Nucleic acid molecules (e.g., polynucleotides, double-stranded nucleic acid molecules, single-stranded nucleic acid molecules, primers, adapters, etc.) may be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety, or phosphate backbone. In some cases, a nucleotide may include a modification in its phosphate moiety, including a modification to a triphosphate moiety. Additional, non-limiting examples of modifications include phosphate chains of greater length (e.g., a phosphate chain having, 4, 5, 6, 7, 8, 9, 10 or more phosphate moieties), modifications with thiol moieties (e.g., alpha-thio triphosphate and beta-thiotriphosphates), and modifications with selenium moieties (e.g., phosphoroselenoate nucleic acids). A nucleotide or nucleotide analog may comprise a sugar selected from the group consisting of ribose, deoxyribose, and modified versions thereof (e.g., by oxidation, reduction, and/or addition of a substituent such as an alkyl, hydroxyalkyl, hydroxyl, or halogen moiety). A nucleotide analog may also comprise a modified linker moiety (e.g., in lieu of a phosphate moiety). Nucleotide analogs may also contain amine-modified groups, such as aminoallyl-dUTP (aa-dUTP) and aminohexylacrylamide-dCTP (aha-dCTP) to allow covalent attachment of amine reactive moieties, such as N-hydroxysuccinimide esters (NHS). Alternatives to standard DNA base pairs or RNA base pairs in the oligonucleotides of the present disclosure may provide, for example, higher density in bits per cubic mm, higher safety (resistant to accidental or purposeful synthesis of natural toxins), easier discrimination in photo-programmed polymerases, and/or lower secondary structure. Nucleotide analogs may be capable of reacting or bonding with detectable moieties for nucleotide detection.

The term “homopolymer,” as used herein, generally refers to a polymer or a portion of a polymer comprising identical monomer units. A homopolymer may have a homopolymer sequence. A nucleic acid homopolymer may refer to a polynucleotide or an oligonucleotide comprising consecutive repetitions of a same nucleotide or any nucleotide variants thereof. For example, a homopolymer can be poly(dA), poly(dT), poly(dG), poly(dC), poly(rA), poly(U), poly(rG), or poly(rC). A homopolymer can be of any length. For example, the homopolymer can have a length of at least 2, 3, 4, 5, 10, 20, 30, 40, 50, 100, 200, 300, 400, 500, or more nucleic acid bases. The homopolymer can have from 10 to 500, or 15 to 200, or 20 to 150 nucleic acid bases. The homopolymer can have a length of at most 500, 400, 300, 200, 100, 50, 40, 30, 20, 10, 5, 4, 3, or 2 nucleic acid bases. A molecule, such as a nucleic acid molecule, can include one or more homopolymer portions and one or more non-homopolymer portions. The molecule may be entirely formed of a homopolymer, multiple homopolymers, or a combination of homopolymers and non-homopolymers. In nucleic acid sequencing, multiple nucleotides can be incorporated into a homopolymeric region of a nucleic acid strand. Such nucleotides may be non-terminated to permit incorporation of consecutive nucleotides (e.g., during a single nucleotide flow).

The terms “amplifying,” “amplification,” and “nucleic acid amplification” are used interchangeably and generally refer to generating one or more copies of a nucleic acid or a template. For example, “amplification” of DNA generally refers to generating one or more copies of a DNA molecule. An amplicon may be a single-stranded or double-stranded nucleic acid molecule that is generated by an amplification procedure from a starting template nucleic acid molecule. Such an amplification procedure may include one or more cycles of an extension or ligation procedure. The amplicon may comprise a nucleic acid strand, of which at least a portion may be substantially identical or substantially complementary to at least a portion of the starting template. Where the starting template is a double-stranded nucleic acid molecule, an amplicon may comprise a nucleic acid strand that is substantially identical to at least a portion of one strand and is substantially complementary to at least a portion of either strand. The amplicon can be single-stranded or double-stranded irrespective of whether the initial template is single-stranded or double-stranded. Amplification of a nucleic acid may linear, exponential, or a combination thereof. Amplification may be emulsion based or may be non-emulsion based. Non-limiting examples of nucleic acid amplification methods include reverse transcription, primer extension, polymerase chain reaction (PCR), ligase chain reaction (LCR), helicase-dependent amplification, asymmetric amplification, rolling circle amplification, and multiple displacement amplification (MDA). Where PCR is used, any form of PCR may be used, with non-limiting examples that include real-time PCR, allele-specific PCR, assembly PCR, asymmetric PCR, digital PCR, emulsion PCR, dial-out PCR, helicase-dependent PCR, nested PCR, hot start PCR, inverse PCR, methylation-specific PCR, miniprimer PCR, multiplex PCR, nested PCR, overlap-extension PCR, thermal asymmetric interlaced PCR, and touchdown PCR. Moreover, amplification can be conducted in a reaction mixture comprising various components (e.g., a primer(s), template, nucleotides, a polymerase, buffer components, co-factors, etc.) that participate or facilitate amplification. In some cases, the reaction mixture comprises a buffer that permits context independent incorporation of nucleotides. Non-limiting examples include magnesium-ion, manganese-ion and isocitrate buffers. Additional examples of such buffers are described in Tabor, S. et al. C. C. PNAS, 1989, 86, 4076-4080 and U.S. Pat. Nos. 5,409,811 and 5,674,716, each of which is herein incorporated by reference in its entirety.

Amplification may be clonal amplification. The term “clonal,” as used herein, generally refers to a population of nucleic acids for which a substantial portion (e.g., greater than about 50%, 60%, 70%, 80%, 90%, 95%, or 99%) of its members have sequences that are at least about 50%, 60%, 70%, 80%, 90%, 95%, or 99% identical to one another. Members of a clonal population of nucleic acid molecules may have sequence homology to one another. Such members may have sequence homology to a template nucleic acid molecule. The members of the clonal population may be double stranded or single stranded. Members of a population may not be 100% identical or complementary, e.g., “errors” may occur during the course of synthesis such that a minority of a given population may not have sequence homology with a majority of the population. For example, at least 50% of the members of a population may be substantially identical to each other or to a reference nucleic acid molecule (i.e., a molecule of defined sequence used as a basis for a sequence comparison). At least 60%, at least 70%, at least 80%, at least 90%, at least 95%, at least 99%, or more of the members of a population may be substantially identical to the reference nucleic acid molecule. Two molecules may be considered substantially identical (or homologous) if the percent identity between the two molecules is at least 60%, 70%, 75%, 80%, 85%, 90%, 95%, 98%, 99%, 99.9% or greater. Two molecules may be considered substantially complementary if the percent complementarity between the two molecules is at least 60%, 70%, 75%, 80%, 85%, 90%, 95%, 98%, 99%, 99.9% or greater. A low or insubstantial level of mixing of non-homologous nucleic acids may occur, and thus a clonal population may contain a minority of diverse nucleic acids (e.g., less than 30%, e.g., less than 10%).

Useful methods for clonal amplification from single molecules include rolling circle amplification (RCA) (Lizardi et al., Nat. Genet. 19:225-232 (1998)), which is incorporated herein by reference), bridge PCR (Adams and Kron, Method for Performing Amplification of Nucleic Acid with Two Primers Bound to a Single Solid Support, Mosaic Technologies, Inc. (Winter Hill, Mass.); Whitehead Institute for Biomedical Research, Cambridge, Mass., (1997); Adessi et al., Nucl. Acids Res. 28:E87 (2000); Pemov et al., Nucl. Acids Res. 33:e11(2005); or U.S. Pat. No. 5,641,658, each of which is incorporated herein by reference), polony generation (Mitra et al., Proc. Natl. Acad. Sci. USA 100:5926-5931 (2003); Mitra et al., Anal. Biochem. 320:55-65(2003), each of which is incorporated herein by reference), and clonal amplification on beads using emulsions (Dressman et al., Proc. Natl. Acad. Sci. USA 100:8817-8822 (2003), which is incorporated herein by reference) or ligation to bead-based adapter libraries (Brenner et al., Nat. Biotechnol. 18:630-634 (2000); Brenner et al., Proc. Natl. Acad. Sci. USA 97:1665-1670 (2000); Reinartz, et al., Brief Funct. Genomic Proteomic 1:95-104 (2002), each of which is incorporated herein by reference). The enhanced signal-to-noise ratio provided by clonal amplification more than outweighs the disadvantages of the cyclic sequencing requirement.

The term “polymerizing enzyme” or “polymerase,” as used herein, generally refers to any enzyme capable of catalyzing a polymerization reaction. A polymerizing enzyme may be used to extend a nucleic acid primer paired with a template strand by incorporation of nucleotides or nucleotide analogs. A polymerizing enzyme may add a new strand of DNA by extending the 3′ end of an existing nucleotide chain, adding new nucleotides matched to the template strand one at a time via the creation of phosphodiester bonds. The polymerase used herein can have strand displacement activity or non-strand displacement activity. Examples of polymerases include, without limitation, a nucleic acid polymerase. An example polymerase is a Φ29 DNA polymerase or a derivative thereof. A polymerase can be a polymerization enzyme. In some cases, a transcriptase or a ligase is used (i.e., enzymes which catalyze the formation of a bond). Examples of polymerases include a DNA polymerase, an RNA polymerase, a thermostable polymerase, a wild-type polymerase, a modified polymerase, E. coli DNA polymerase I, T7 DNA polymerase, bacteriophage T4 DNA polymerase Φ29 (phi29) DNA polymerase, Taq polymerase, Tth polymerase, Tli polymerase, Pfu polymerase, Pwo polymerase, VENT polymerase, DEEPVENT polymerase, EX-Taq polymerase, LA-Taq polymerase, Sso polymerase, Poc polymerase, Pab polymerase, Mth polymerase, ES4 polymerase, Tru polymerase, Tac polymerase, Tne polymerase, Tma polymerase, Tea polymerase, Tih polymerase, Tfi polymerase, Platinum Taq polymerases, Tbr polymerase, Tfl polymerase, Pfu-turbo polymerase, Pyrobest polymerase, Pwo polymerase, KOD polymerase, Bst polymerase, Sac polymerase, Klenow fragment, polymerase with 3′ to 5′ exonuclease activity, and variants, modified products and derivatives thereof. In some cases, the polymerase is a single subunit polymerase. The polymerase can have high processivity, namely the capability of the polymerase to consecutively incorporate nucleotides into a nucleic acid template without releasing the nucleic acid template. In some cases, a polymerase is a polymerase modified to accept dideoxynucleotide triphosphates, such as for example, Taq polymerase having a 667Y mutation (see e.g., Tabor et al, PNAS, 1995, 92, 6339-6343, which is herein incorporated by reference in its entirety for all purposes). In some cases, a polymerase is a polymerase having a modified nucleotide binding, which may be useful for nucleic acid sequencing, with non-limiting examples that include ThermoSequenas polymerase (GE Life Sciences), AmpliTaq FS (ThermoFisher) polymerase and Sequencing Pol polymerase (Jena Bioscience). In some cases, the polymerase is genetically engineered to have discrimination against dideoxynucleotides, such as for example, Sequenase DNA polymerase (ThermoFisher).

A polymerase may be Family A polymerase or a Family B DNA polymerase. Family A polymerases include, for example, Taq, Klenow, and Bst polymerases. Family B polymerases include, for example, Vent(exo-) and Therminator™ polymerases. Family B polymerases are known to accept more varied nucleotide substrates than Family A polymerases. Family A polymerases are used widely in sequencing by synthesis methods, likely due to their high processivity and fidelity.

The term “complementary sequence,” as used herein, generally refers to a sequence that hybridizes to another sequence. Hybridization between two single-stranded nucleic acid molecules may involve the formation of a double-stranded structure that is stable under certain conditions. Two single-stranded polynucleotides may be considered to be hybridized if they are bonded to each other by two or more sequentially adjacent base pairings. A substantial proportion of nucleotides in one strand of a double-stranded structure may undergo Watson-Crick base-pairing with a nucleoside on the other strand. Hybridization may also include the pairing of nucleoside analogs, such as deoxyinosine, nucleosides with 2-aminopurine bases, and the like, that may be employed to reduce the degeneracy of probes, whether or not such pairing involves formation of hydrogen bonds.

The term “denaturation,” as used herein, generally refers to separation of a double-stranded molecule (e.g., DNA) into single-stranded molecules. Denaturation may be complete or partial denaturation. In partial denaturation, a single-stranded region may form in a double-stranded molecule by denaturation of the two deoxyribonucleic acid (DNA) strands flanked by double-stranded regions in DNA.

The term “melting temperature” or “melting point,” as used herein, generally refers to the temperature at which at least a portion of a strand of a nucleic acid molecule in a sample has separated from at least a portion of a complementary strand. The melting temperature may be the temperature at which a double-stranded nucleic acid molecule has partially or completely denatured. The melting temperature may refer to a temperature of a sequence among a plurality of sequences of a given nucleic acid molecule, or a temperature of the plurality of sequences. Different regions of a double-stranded nucleic acid molecule may have different melting temperatures. For example, a double-stranded nucleic acid molecule may include a first region having a first melting point and a second region having a second melting point that is higher than the first melting point. Accordingly, different regions of a double-stranded nucleic acid molecule may melt (e.g., partially denature) at different temperatures. The melting point of a nucleic acid molecule or a region thereof (e.g., a nucleic acid sequence) may be determined experimentally (e.g., via a melt analysis or other procedure) or may be estimated based upon the sequence and length of the nucleic acid molecule. For example, a software program such as MELTING may be used to estimate a melting temperature for a nucleic acid sequence (Dumousseau M, Rodriguez N, Juty N, Le Novere N, MELTING, a flexible platform to predict the melting temperatures of nucleic acids. BMC Bioinformatics. 2012 May 16; 13:101. doi: 10.1186/1471-2105-13-101). Accordingly, a melting point as described herein may be an estimated melting point. A true melting point of a nucleic acid sequence may vary based upon the sequences or lack thereof adjacent to the nucleic acid sequence of interest as well as other factors.

The term “sequencing,” as used herein, generally refers to a process for generating or identifying a sequence of a biological molecule, such as a nucleic acid molecule or a polypeptide. Such sequence may be a nucleic acid sequence, which may include a sequence of nucleic acid bases (e.g., nucleobases). Sequencing may be, for example, single molecule sequencing, sequencing by synthesis, sequencing by hybridization, or sequencing by ligation. Sequencing may be performed using template nucleic acid molecules immobilized on a support, such as a flow cell or one or more beads. A sequencing assay may yield one or more sequencing reads corresponding to one or more template nucleic acid molecules.

The term “read,” as used herein, generally refers to a nucleic acid sequence, such as a sequencing read. A sequencing read may be an inferred sequence of nucleic acid bases (e.g., nucleotides) or base pairs obtained via a nucleic acid sequencing assay. A sequencing read may be generated by a nucleic acid sequencer, such as a massively parallel array sequencer (e.g., Illumina or Pacific Biosciences of California). A sequencing read may correspond to a portion, or in some cases all, of a genome of a subject. A sequencing read may be part of a collection of sequencing reads, which may be combined through, for example, alignment (e.g., to a reference genome), to yield a sequence of a genome of a subject.

The term “detector,” as used herein, generally refers to a device that is capable of detecting or measuring a signal, such as a signal indicative of the presence or absence of an incorporated nucleotide or nucleotide analog. A detector may include optical and/or electronic components that may detect and/or measure signals. Non-limiting examples of detection methods involving a detector include optical detection, spectroscopic detection, electrostatic detection, and electrochemical detection. Optical detection methods include, but are not limited to, fluorimetry and UV-vis light absorbance. Spectroscopic detection methods include, but are not limited to, mass spectrometry, nuclear magnetic resonance (NMR) spectroscopy, and infrared spectroscopy. Electrostatic detection methods include, but are not limited to, gel-based techniques, such as, for example, gel electrophoresis. Electrochemical detection methods include, but are not limited to, electrochemical detection of amplified product after high-performance liquid chromatography separation of the amplified products.

The term “support,” as used herein, generally refers to any solid or semi-solid article on which reagents such as nucleic acid molecules may be immobilized. Nucleic acid molecules may be synthesized, attached, ligated, or otherwise immobilized. Nucleic acid molecules may be immobilized on a support by any method including, but not limited to, physical adsorption, by ionic or covalent bond formation, or combinations thereof. A support may be 2-dimensional (e.g., a planar 2D support) or 3-dimensional. In some cases, a support may be a component of a flow cell and/or may be included within or adapted to be received by a sequencing instrument. A support may include a polymer, a glass, or a metallic material. Examples of supports include a membrane, a planar support, a microtiter plate, a bead (e.g., a magnetic bead), a filter, a test strip, a slide, a cover slip, and a test tube. A support may comprise organic polymers such as polystyrene, polyethylene, polypropylene, polyfluoroethylene, polyethyleneoxy, and polyacrylamide (e.g., polyacrylamide gel), as well as co-polymers and grafts thereof. A support may comprise latex or dextran. A support may also be inorganic, such as glass, silica, gold, controlled-pore-glass (CPG), or reverse-phase silica. The configuration of a support may be, for example, in the form of beads, spheres, particles, granules, a gel, a porous matrix, or a support. In some cases, a support may be a single solid or semi-solid article (e.g., a single particle), while in other cases a support may comprise a plurality of solid or semi-solid articles (e.g., a collection of particles). Supports may be planar, substantially planar, or non-planar. Supports may be porous or non-porous and may have swelling or non-swelling characteristics. A support may be shaped to comprise one or more wells, depressions, or other containers, vessels, features, or locations. A plurality of supports may be configured in an array at various locations. A support may be addressable (e.g., for robotic delivery of reagents), or by detection approaches, such as scanning by laser illumination and confocal or deflective light gathering. For example, a support may be in optical and/or physical communication with a detector. Alternatively, a support may be physically separated from a detector by a distance. An amplification support (e.g., a bead) can be placed within or on another support (e.g., within a well of a second support). A nucleic acid or an enzyme may be immobilized to a support (e.g., by a streptavidin-biotin linkage or by a streptactin-strep tag interaction). A nucleic acid immobilized to a support may undergo extension (e.g., chemical extension or polymerase mediated extension), or may serve as a template for a second nucleic acid which undergoes extension.

The term “label,” as used herein, generally refers to a moiety that is capable of coupling with a species, such as, for example a nucleotide analog. A label may include an affinity moiety. In some cases, a label may be a detectable label that emits a signal (or reduces an already emitted signal) that can be detected. In some cases, such a signal may be indicative of incorporation of one or more nucleotides or nucleotide analogs. In some cases, a label may be coupled to a nucleotide or nucleotide analog, which nucleotide or nucleotide analog may be used in a primer extension reaction. The label may be coupled to a nucleobase of the nucleotide or nucleotide analog. The label may be coupled to a sugar (e.g., ribosyl moiety) of the nucleotide or nucleotide analog. The label may be coupled to a phosphate of the nucleotide or nucleotide analog. In some cases, the label may be coupled to a nucleotide analog after a primer extension reaction. The label, in some cases, may be reactive specifically with a nucleotide or nucleotide analog. Coupling may be covalent or non-covalent (e.g., via ionic interactions, Van der Waals forces, etc.). In some cases, coupling may be via a linker, which may be cleavable, such as photo-cleavable (e.g., cleavable under ultra-violet light), chemically-cleavable (e.g., via a reducing agent, such as dithiothreitol (DTT), tris(2-carboxyethyl)phosphine (TCEP), tris(hydroxypropyl)phosphine (THP) or enzymatically cleavable (e.g., via an esterase, lipase, peptidase, or protease).

A label may be detectable. A label may comprise a detectable moiety. In some cases, the label or detectable moiety may be optically detectable. The label or detectable moiety may be luminescent; for example, fluorescent, phosphorescent, or chemiluminescent. The label or detectable moiety may comprise a dye (e.g., a fluorescent moiety). The label or detectable moiety may absorb, disperse, polarize, or rotate light, which may enable identification of the label. For example, a label or detectable moiety may comprise a characteristic absorbance band that enables its detection in a complex sample. In some cases, the label or detectable moiety may be electrochemically detectable. For example, the label may comprise an electrochemically detectable moiety, which may comprise a characteristic oxidation or reduction potential that may be used to identify the label. A label may comprise a mass tag, which may enable mass spectrometric identification of the label. A plurality of labels may comprise a plurality of different detectable moieties. For example, each label from among a set of four labels may comprise a different fluorescent dye.

Dyes and labels may be incorporated into nucleic acid sequences. Dyes and labels may also be incorporated into or attached to linkers, such as linkers for linking one or more beads to one another. For example, labels such as fluorescent moieties may be linked to nucleotides or nucleotide analogs via a linker (e.g., as described herein). Non-limiting examples of dyes include SYBR green, SYBR blue, DAPI, propidium iodine, Hoechst, SYBR gold, ethidium bromide, acridine, proflavine, acridine orange, acriflavine, fluorcoumanin, ellipticine, daunomycin, chloroquine, distamycin D, chromomycin, homidium, mithramycin, ruthenium polypyridyls, anthramycin, phenanthridines and acridines, propidium iodide, hexidium iodide, dihydroethidium, ethidium homodimer-1 and -2, ethidium monoazide, ACMA, Hoechst 33258, Hoechst 33342, Hoechst 34580, DAPI, acridine orange, 7-AAD, actinomycin D, LDS751, hydroxystilbamidine, SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO labels (e.g., SYTO-40, -41, -42, -43, -44, and -45 (blue); SYTO-13, -16, -24, -21, -23, -12, -11, -20, -22, -15, -14, and -25 (green); SYTO-81, -80, -82, -83, -84, and -85 (orange); and SYTO-64, -17, -59, -61, -62, -60, and -63 (red)), fluorescein, fluorescein isothiocyanate (FITC), tetramethyl rhodamine isothiocyanate (TRITC), rhodamine, tetramethyl rhodamine, R-phycoerythrin, Cy-2, Cy-3, Cy-3.5, Cy-5, Cy5.5, Cy-7, Texas Red, Phar-Red, allophycocyanin (APC), Sybr Green I, Sybr Green II, Sybr Gold, CellTracker Green, 7-AAD, ethidium homodimer I, ethidium homodimer II, ethidium homodimer III, ethidium bromide, umbelliferone, eosin, green fluorescent protein, erythrosin, coumarin, methyl coumarin, pyrene, malachite green, stilbene, lucifer yellow, cascade blue, dichlorotriazinylamine fluorescein, dansyl chloride, fluorescent lanthanide complexes such as those including europium and terbium, carboxy tetrachloro fluorescein, 5 and/or 6-carboxy fluorescein (FAM), VIC, 5- (or 6-) iodoacetamidofluorescein, 5-{[2(and 3)-5-(Acetylmercapto)-succinyl]amino} fluorescein (SAMSA-fluorescein), lissamine rhodamine B sulfonyl chloride, 5 and/or 6 carboxy rhodamine (ROX), 7-amino-methyl-coumarin, 7-Amino-4-methylcoumarin-3-acetic acid (AMCA), BODIPY fluorophores, 8-methoxypyrene-1,3,6-trisulfonic acid trisodium salt, 3,6-Disulfonate-4-amino-naphthalimide, phycobiliproteins, AlexaFluor labels (e.g., AlexaFluor 350, 405, 430, 488, 532, 546, 555, 568, 594, 610, 633, 635, 647, 660, 680, 700, 750, and 790 dyes), DyLight labels (e.g., DyLight 350, 405, 488, 550, 594, 633, 650, 680, 755, and 800 dyes), Black Hole Quencher Dyes (Biosearch Technologies) (e.g., BH1-0, BHQ-1, BHQ-3, and BHQ-10), QSY Dye fluorescent quenchers (Molecular Probes/Invitrogen) (e.g., QSY7, QSY9, QSY21, and QSY35), Dabcyl, Dabsyl, Cy5Q, Cy7Q, Dark Cyanine dyes (GE Healthcare), Dy-Quenchers (Dyomics) (e.g., DYQ-660 and DYQ-661), ATTO fluorescent quenchers (ATTO-TEC GmbH) (e.g., ATTO 540Q, ATTO 580Q, ATTO 612Q, Atto532 [e.g., Atto 532 succinimidyl ester], and Atto633), and other fluorophores and/or quenchers. Additional examples are included in structures provided herein. Dyes included in structures provided herein are contemplated for use in combination with any linker and substrate described herein. A fluorescent dye may be excited by application of energy corresponding to the visible region of the electromagnetic spectrum (e.g., between about 430-770 nanometers (nm)). Excitation may be done using any useful apparatus, such as a laser and/or light emitting diode. Optical elements including, but not limited to, mirrors, waveplates, filters, monochromators, gratings, beam splitters, and lenses may be used to direct light to or from a fluorescent dye. A fluorescent dye may emit light (e.g., fluoresce) in the visible region of the electromagnetic spectrum ((e.g., between about 430-770 nm). A fluorescent dye may be excited over a single wavelength or a range of wavelengths. A fluorescent dye may be excitable by light in the red region of the visible portion of the electromagnetic spectrum (about 625-740 nm) (e.g., have an excitation maximum in the red region of the visible portion of the electromagnetic spectrum). Alternatively or in addition to, fluorescent dye may be excitable by light in the green region of the visible portion of the electromagnetic spectrum (about 500-565 nm) (e.g., have an excitation maximum in the green region of the visible portion of the electromagnetic spectrum). A fluorescent dye may emit signal in the red region of the visible portion of the electromagnetic spectrum (about 625-740 nm) (e.g., have an emission maximum in the red region of the visible portion of the electromagnetic spectrum). Alternatively or in addition to, fluorescent dye may emit signal in the green region of the visible portion of the electromagnetic spectrum (about 500-565 nm) (e.g., have an emission maximum in the green region of the visible portion of the electromagnetic spectrum).

Labels may be quencher molecules. The term “quencher,” as used herein, generally refers to molecules that may be energy acceptors. A quencher may be a molecule that can reduce an emitted signal. For example, a template nucleic acid molecule may be designed to emit a detectable signal. Incorporation of a nucleotide or nucleotide analog comprising a quencher can reduce or eliminate the signal, which reduction or elimination is then detected. Luminescence from labels (e.g., fluorescent moieties, such as fluorescent moieties linked to nucleotides or nucleotide analogs) may also be quenched (e.g., by incorporation of other nucleotides that may or may not comprise labels). In some cases, as described elsewhere herein, labelling with a quencher can occur after nucleotide or nucleotide analog incorporation (e.g., after incorporation of a nucleotide or nucleotide analog comprising a fluorescent moiety). In some cases, the label may be a type that does not self-quench or exhibit proximity quenching. Non-limiting examples of a label type that does not self-quench or exhibit proximity quenching include Bimane derivatives such as Monobromobimane. The term “proximity quenching,” as used herein, generally refers to a phenomenon where one or more dyes near each other may exhibit lower fluorescence as compared to the fluorescence they exhibit individually. In some cases, the dye may be subject to proximity quenching wherein the donor dye and acceptor dye are within 1 nm to 50 nm of each other. Examples of quenchers include, but are not limited to, Black Hole Quencher Dyes (Biosearch Technologies) (e.g., BH1-0, BHQ-1, BHQ-3, and BHQ-10), QSY Dye fluorescent quenchers (Molecular Probes/Invitrogen) (e.g., QSY7, QSY9, QSY21, and QSY35), Dabcyl, Dabsyl, Cy5Q, Cy7Q, Dark Cyanine dyes (GE Healthcare), Dy-Quenchers (Dyomics) (e.g., DYQ-660 and DYQ-661), and ATTO fluorescent quenchers (ATTO-TEC GmbH) (e.g., ATTO 540Q, ATTO 580Q, and ATTO 612Q). Fluorophore donor molecules may be used in conjunction with a quencher. Examples of fluorophore donor molecules that can be used in conjunction with quenchers include, but are not limited to, fluorophores such as Cy3B, Cy3, or Cy5; Dy-Quenchers (Dyomics) (e.g., DYQ-660 and DYQ-661); and ATTO fluorescent quenchers (ATTO-TEC GmbH) (e.g., ATTO 540Q, 580Q, and 612Q).

The term “labeling fraction,” as used herein, generally refers to the ratio of dye-labeled nucleotide or nucleotide analog to natural/unlabeled nucleotide or nucleotide analog of a single canonical type in a flow solution. The labeling fraction can be expressed as the concentration of the labeled nucleotide or nucleotide analog divided by the sum of the concentrations of labeled and unlabeled nucleotide or nucleotide analog. The labeling fraction may be expressed as a % of labeled nucleotides included in a solution (e.g., a nucleotide flow). The labeling fraction may be at least about 0.5%, 1%, 2%, 3%, 4%, 5%, 10%, 15%, 20%, 30%, 40%, 50%, 60%, 70%, 80%, 90%, or higher. For example, the labeling fraction may be at least about 20%. The labeling fraction may be about 100%. The labeling fraction may also be expressed as a ratio of labeled nucleotides to unlabeled nucleotides included in a solution. For example, the ratio of labeled nucleotides to unlabeled nucleotides may be at least about 1:5, 1:4, 1:3, 1:2, 1:1, 2:1, 3:1, 4:1, 5:1, or higher. For example, the ratio of labeled nucleotides to unlabeled nucleotides may be at least about 1:4. For example, the ratio of labeled nucleotides to unlabeled nucleotides may be at least about 1:1. For example, the ratio of labeled nucleotides to unlabeled nucleotides may be at least about 5:1.

The term “labeled fraction,” as used herein, generally refers to the actual fraction of labeled nucleic acid (e.g., DNA) resulting after treatment of a primer-template with a mixture of the dye-labeled and natural nucleotide or nucleotide analog. The labeled fraction may be about the same as the labeling fraction. For example, if 20% of nucleotides in a nucleotide flow are labeled, about 20% of nucleotides incorporated into a growing nucleic acid strand (e.g., during nucleic acid sequencing) may be labeled. Alternatively, the labeled fraction may be greater than the labeled fraction. For example, if 20% of nucleotides in a nucleotide flow are labeled, greater than 20% of nucleotides incorporated into a growing nucleic acid strand (e.g., during nucleic acid sequencing) may be labeled. Alternatively, the labeled fraction may be less than the labeled fraction. For example, if 20% of nucleotides in a nucleotide flow are labeled, less than 20% of nucleotides incorporated into a growing nucleic acid strand (e.g., during nucleic acid sequencing) may be labeled.

When a solution including less than 100% labeled nucleotides or nucleotide analogs is used in an incorporation process such as a sequencing process (e.g., as described herein), both labeled (“bright”) and unlabeled (“dark”) nucleotides or nucleotide analogs may be incorporated into a growing nucleic acid strand. The term “tolerance,” as used herein, generally refers to the ratio of the labeled fraction (e.g., “bright” incorporated fraction) to the labeling fraction (e.g., “bright” fraction in solution). For example, if a labeling fraction of 0.2 is used resulting in a labeled fraction of 0.4 the tolerance is 2. Similarly, if an incorporation process such as a sequencing process is performed using 2.5% labeled fraction in solution (b_{f}, bright solution fraction) and 5% is labeled (bᵢ, bright incorporated fraction), the tolerance may be 2 (e.g., tolerance). This model may be linear for low labeling fractions (e.g., 10% or lower labeling fraction). For higher labeling fractions, tolerance may take into account competing dark incorporation. Tolerance may refer to a comparison of the ratio of bright incorporated fraction to dark incorporated fraction (bᵢ/dᵢ) to the ratio of bright solution fraction to dark solution fraction (b_{f}/d_{f}):

$${b}_{i} = \frac{tol ( {b}_{f} / {d}_{f} )}{1 + tol ( {b}_{f} / {d}_{f} )}$$

where dᵢ=1−bᵢ (e.g., dark incorporated fraction and bright incorporated fraction sum to 1 assuming 100% bright fraction is normalized to 1)

Though dᵢ cannot easily be measured, bᵢ, the bright incorporated fraction, can be measured (e.g., as described herein) and used to determine tolerance (tol) by fitting a curve of bright solution fraction (b_{f}) vs. bright incorporated fraction (bᵢ):

$$Tolerance = \frac{{b}_{i} / {d}_{i}}{{b}_{f} / {d}_{f}}$$

A “positive” tolerance number (>1) indicates that at 50% labeling fraction, more than 50% is labeled. A “negative” tolerance number (<1) indicates that at 50% labeling fraction, less than 50% is labeled.

The term “context,” as used herein, generally refers to the sequence of the neighboring nucleotides, or context, has been observed to affect the tolerance in an incorporation reaction. The nature of the enzyme, the pH and other factors may also affect the tolerance. Reducing context effects to a minimum greatly simplifies base determination.

The term “scar,” as used herein, generally refers to a residue left on a previously labeled nucleotide or nucleotide analog after cleavage of an optical (e.g., fluorescent) dye and, optionally, all or a portion of a linker attaching the optical dye to the nucleotide or nucleotide analog. Examples of scars include, but are not limited to, hydroxyl moieties (e.g., resulting from cleavage of an azidomethyl group, hydrocarbyldithiomethyl linkage, or 2-nitrobenzyloxy linkage), thiol moieties (e.g., resulting from cleavage of a disulfide linkage), propargyl moieties (e.g., propargyl alcohol, propargyl amine, or propargyl thiol), and benzyl moieties. For example, a scar may comprise an aromatic group such as a phenyl or benzyl group. The size and nature of a scar may affect subsequent incorporations.

The term “misincorporation,” as used herein, generally refers to occurrences when the DNA polymerase incorporates a nucleotide, either labeled or unlabeled, that is not the correct Watson-Crick partner for the template base. Misincorporation can occur more frequently in methods that lack competition of all four bases in an incorporation event, and leads to strand loss, and thus limits the read length of a sequencing method.

The term “mispair extension,” as used herein, generally refers to occurrences when the DNA polymerase incorporates a nucleotide, either labeled or unlabeled, that is not the correct Watson-Crick partner for the template base, then subsequently incorporates the correct Watson-Crick partner for the following base. Mispair extension generally results in lead phasing and limits the read length of a sequencing method.

Regarding quenching, dye-dye quenching between two dye moieties linked to different nucleotides (e.g., adjacent nucleotides in a growing nucleic acid strand, or nucleotides in a nucleic acid strand that are separated by one or more other nucleotides) may be strongly dependent on the distance between the two dye moieties. The distance between two dye moieties may be at least partially dependent on the properties of linkers connecting the two dye moieties to respective nucleotides or nucleotide analogs, including the linker compositions and functional lengths. Features of the linkers, including composition and functional length, may be affected by temperature, solvent, pH, and salt concentration (e.g., within a solution). Quenching may also vary based on the nature of the dyes used. Quenching may also take place between dye moieties and nucleobase moieties (e.g., between a fluorescent dye and a nucleobase of a nucleotide with which it is associated). Controlling quenching phenomena may be a key feature of the methods described herein.

Regarding flows, a nucleotide flow can consist of a mixture of labeled and unlabeled nucleotides or nucleotide analogs (e.g., nucleotides or nucleotide analogs of a single canonical type). For example, a solution comprising a plurality of optically (e.g., fluorescently) labeled nucleotides and a plurality of unlabeled nucleotides may be contacted with, e.g., a sequencing template (as described herein). The plurality of optically labeled nucleotides and a plurality of unlabeled nucleotides may each comprise the same canonical nucleotide or nucleotide analog. A flow may include only labeled nucleotides or nucleotide analogs. Alternatively, a flow may include only unlabeled nucleotides or nucleotide analogs. A flow may include a mixture of nucleotide or nucleotide analogs of different types (e.g., A and G).

A wash flow (e.g., a solution comprising a buffer) may be used to remove any nucleotides that are not incorporated into a nucleic acid complex (e.g., a sequencing template, as described herein). A cleavage flow (e.g., a solution comprising a cleavage reagent) may be used to remove dye moieties (e.g., fluorescent dye moieties) from optically (e.g., fluorescently) labeled nucleotides or nucleotide analogs. In some cases, different dyes (e.g., fluorescent dyes) may be removable using different cleavage reagents. In other cases, different dyes (e.g., fluorescent dyes) may be removable using the same cleavage reagents. Cleavage of dye moieties from optically labeled nucleotides or nucleotide analogs may comprise cleavage of all or a portion of a linker connecting a nucleotide or nucleotide analog to a dye moiety.

The term “cycle,” as used herein, generally refers to a process in which a nucleotide flow, a wash flow, and a cleavage flow corresponding to each canonical nucleotide (e.g., dATP, dCTP, dGTP, and dTTP or dUTP, or modified versions thereof) are used (e.g., provided to a sequencing template, as described herein). Multiple cycles may be used to sequence and/or amplify a nucleic acid molecule. The order of nucleotide flows can be varied.

Phasing can be lead or lag phasing. Lead phasing generally refers to the phenomenon in which a population of strands show incorporation of a nucleotide a flow ahead of the expected cycle (e.g., due to contamination in the system). Lag phasing refers to the phenomenon in which a population of strands shows incorporation of a nucleotide a flow behind the expected cycle (e.g., due to incompletion of extension in an earlier cycle).

Compounds and chemical moieties described herein, including linkers, may contain one or more asymmetric centers and thus give rise to enantiomers, diastereomers, and other stereoisomeric forms that are defined, in terms of absolute stereochemistry, as (R)- or (S)-, and, in terms of relative stereochemistry, as (D)- or (L)-. The D/L system relates molecules to the chiral molecule glyceraldehyde and is commonly used to describe biological molecules including amino acids. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a phenyl ring. Separation of stereoisomers may be performed by chromatography or by forming diastereomers and separating by recrystallization, or chromatography, or any combination thereof. (Jean Jacques, Andre Collet, Samuel H. Wilen, “Enantiomers, Racemates and Resolutions,” John Wiley and Sons, Inc., 1981, herein incorporated by reference for this disclosure). Stereoisomers may also be obtained by stereoselective synthesis.

Compounds and chemical moieties described herein, including linkers, may exist as tautomers. A “tautomer” refers to a molecule wherein a proton shift from one atom of a molecule to another atom of the same molecule is possible. In circumstances where tautomerization is possible, a chemical equilibrium of the tautomers will exist. Unless otherwise stated, chemical structures depicted herein are intended to include structures which are different tautomers of the structures depicted. For example, the chemical structure depicted with an enol moiety also includes the keto tautomer form of the enol moiety. The exact ratio of the tautomers depends on several factors, including physical state, temperature, solvent, and pH. Some examples of tautomeric equilibrium include:

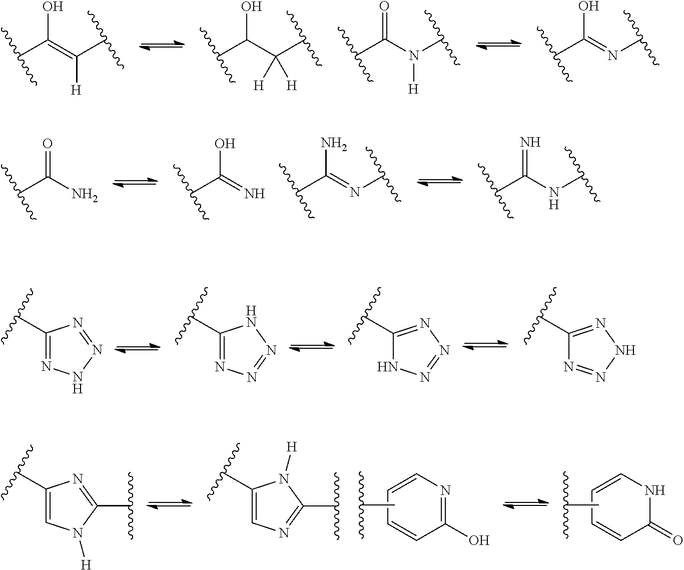

Compounds and chemical moieties described herein, including linkers and dyes, may be provided in different enriched isotopic forms. For example, compounds may be enriched in the content of ²H, ³H, ¹¹C, ¹³C and/or ¹⁴C. For example, a linker, substrate (e.g., nucleotide or nucleotide analog), or dye may be deuterated in at least one position. In some examples, a linker, substrate (e.g., nucleotide or nucleotide analog), or dye may be fully deuterated. Such deuterated forms can be made by the procedure described in U.S. Pat. Nos. 5,846,514 and 6,334,997, each of which are herein incorporated by reference in their entireties. As described in U.S. Pat. Nos. 5,846,514 and 6,334,997, deuteration can improve the metabolic stability and or efficacy, thus increasing the duration of action of drugs.

Unless otherwise stated, structures depicted and described herein are intended to include compounds which differ only in the presence of one or more isotopically enriched atoms. For example, compounds and chemical moieties having the present structures except for the replacement of a hydrogen by a deuterium or tritium, or the replacement of a carbon by ¹³C- or ¹⁴C-enriched carbon are within the scope of the present disclosure.

The compounds and chemical moieties of the present disclosure may contain unnatural proportions of atomic isotopes at one or more atoms that constitute such compounds. For example, a compound or chemical moiety such as a linker, substrate (e.g., nucleotide or nucleotide analog), or dye, or a combination thereof, may be labeled with one or more isotopes, such as deuterium (²H), tritium (H), iodine-125 (¹²⁵I) or carbon-14 (¹⁴C). Isotopic substitution with ²H, ¹¹C, ¹³C, ¹⁴C, ¹⁵C, ¹²N ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, and ¹²⁵I are all contemplated. All isotopic variations of the compounds and chemical moieties described herein, whether radioactive or not, are encompassed within the scope of the present disclosure.

### Linkers for Optical Detection

The present disclosure provides an optical (e.g., fluorescent) labeling reagent comprising a dye (e.g., fluorescent dye) and a linker that is connected to the dye and capable of associating with a substrate to be optically (e.g., fluorescently) labeled. The substrate can be any suitable molecule, analyte, cell, tissue, or surface that is to be optically labeled. Examples include cells, including eukaryotic cells, prokaryotic cells, healthy cells, and diseased cells; cellular receptors; antibodies; proteins; lipids; saccharides (e.g., oligosaccharides and polysaccharides); metabolites; probes; reagents; nucleotides and analogues thereof, and nucleic acid molecules. In some cases, a substrate is selected from the group consisting of a nucleotide, a protein, a lipid, a cell, a polysaccharide, and an antibody. In some cases, a substrate comprises a nucleotide. In some cases, the substrate is isolated from a biological sample, such as a blood, plasma, tissue, urine, or buccal swabbing. For example, the substrate may comprise a circulating tumor cell isolated from a blood sample. The association between the linker and the substrate can be any suitable association including a covalent or non-covalent bond, such as an association between a purine-containing nucleotide and a pyrimidine-containing nucleotide in a nucleic acid molecule. In some cases, such an association may be a biotin-streptavidin interaction. In other cases, the association between the linker and the substrate may be via a propargylamino moiety. In some cases, the association between the linker and the substrate may be via an amide bond (e.g., a peptide bond).

A substrate may be a species configured to bind to an enzyme (e.g., bind to an enzyme active site). A substrate may be a species configured to be acted upon by an enzyme. For example, a substrate may be configured to act as a reagent for an enzyme catalyzed reaction, such as a DNA polymerization reaction. The reaction may be an intermolecular reaction or an intermolecular reaction. The substrate may comprise a substrate configured to be acted upon by a polymerase, a transcriptase, a reverse transcriptase, or any combination thereof.

A linker can be semi-rigid. The semi-rigid nature of the linker can be most readily achieved by use of structure that comprises a series of ring systems (e.g., aliphatic and aromatic rings). As used herein, a ring (e.g., ring structure) is a cyclic moiety comprising any number of atoms connected in a closed, essentially circular fashion, as used in the field of organic chemistry. A ring may be defined by any number of atoms. For example, a ring may include between 3-12 atoms, such as between 3-12 carbon atoms. In certain examples, a ring may be a five-membered ring (i.e., a pentagon) or a six-membered ring (i.e., a hexagon). A ring can be aromatic or non-aromatic. A ring may be aliphatic. A ring may comprise one or more double bonds.

A ring (e.g., ring structure) may be a component of a ring system that may comprise one or more ring structures (e.g., a multi-cycle system). For example, a ring system may comprise a monocycle. In another example, a ring system may be a bicycle or bridged system. A ring structure may be a carbocycle or component thereof formed of carbon atoms. A carbocycle may be a saturated, unsaturated, or aromatic ring in which each atom of the ring is carbon. A carbocycle includes 3- to 10-membered monocyclic rings, 4- to 12-membered bicyclic rings (e.g., 6- to 12-membered bicyclic rings), and 5- to 12-membered bridged rings. Each ring of a bicyclic carbocycle may be selected from saturated, unsaturated, and aromatic rings. For example, a bicyclic carbocycle may include an aromatic ring (e.g., phenyl) fused to a saturated or unsaturated ring (e.g., cyclohexane, cyclopentane, or cyclohexene). A bicyclic carbocycle may include any combination of saturated, unsaturated, and aromatic bicyclic rings, as valence permits. A bicyclic carbocycle may include any combination of ring sizes such as 4-5 fused ring systems, 5-5 fused ring systems, 5-6 fused ring systems, and 6-6 fused ring systems. A carbocycle may be, for example, cyclopropyl, cyclobutyl, cyclopentyl, cyclohexyl, cyclohexenyl, adamantyl, phenyl, indanyl, or naphthyl. A saturated carbocycle includes no multiple bonds (e.g., double or triple bonds). A saturated carbocycle may be, for example, cyclopropane, cyclobutane, cyclopentane, or cyclohexane. An unsaturated carbocycle includes at least one multiple bond (e.g., a double or triple bond) but is not an aromatic carbocycle. An unsaturated carbocycle may be, for example, cyclohexadiene, cyclohexene, or cyclopentene. Other examples of carbocycles include, but are not limited to, cyclopropane, cyclobutane, cyclopentane, cyclopentadiene, cyclohexane, cycloheptane, cycloheptene, naphthalene, and adamantine. An aromatic carbocycle (e.g., aryl moiety) may be, for example, phenyl, naphthyl, or dihydronaphthyl.

In some cases, a ring may include one or more heteroatoms, such as one or more oxygen, nitrogen, silicon, phosphorous, boron, or sulfur atoms. A ring may be a heterocycle or component thereof including one or more heteroatoms. A heterocycle may be a saturated, unsaturated, or aromatic ring in which at least one atom is a heteroatom. A heteroatom includes 3- to 10-membered monocyclic rings, 6- to 12-membered bicyclic rings, and 6- to 12-membered bridged rings. A bicyclic heterocycle may include any combination of saturated, unsaturated, and aromatic bicyclic rings, as valence permits. For example, a heteroaromatic ring (e.g., pyridyl) may be fused to a saturated or unsaturated ring (e.g., cyclohexane, cyclopentane, morpholine, piperidine or cyclohexene). A bicyclic heterocycle may include any combination of ring sizes such as 4-5 fused ring systems, 5-5 fused ring systems, 5-6 fused ring systems, and 6-6 fused ring systems. An unsaturated heterocycle includes at least one multiple bond (e.g., a double or triple bond) but is not an aromatic heterocycle. An unsaturated heterocycle may be, for example, dihydropyrrole, dihydrofuran, oxazoline, pyrazoline, or dihydropyridine. Additional examples of heterocycles include, but are not limited to, indole, benzothiophene, benzothiazole, benzoxazole, benzimidazole, oxazolopyridine, imidazopyridine, thiazolopyridine, furan, oxazole, pyrrole, pyrazole, imidazole, thiophene, thiazole, isothiazole, and isoxazole. A heteroaryl moiety may be an aromatic single ring structure, such as a 5- to 7-membered ring, including at least one heteroatom, such as one to four heteroatoms. Alternatively, a heteroaryl moiety may be a polycyclic ring system having two or more cyclic rings in which two or more atoms are common to two adjoining rings wherein at least one of the rings is heteroaromatic. Heteroaryl groups include, for example, pyrrole, furan, thiophene, imidazole, oxazole, thiazole, pyrazole, pyridine, pyrazine, pyridazine, and pyrimidine, and the like.

A ring can be substituted or un-substituted. A substituent replaces a hydrogen atom on one or more atoms of a ring or a substitutable heteroatom of a ring (e.g., NH or NH₂). Substitution is in accordance with permitted valence of the various components of the ring system and provides a stable compound (e.g., a compound that does not undergo spontaneous transformation by, for example, rearrangement, elimination, or cyclization). A substituent may replace a single hydrogen atom or multiple hydrogen atoms (e.g., on the same ring atom or different ring atoms). A substituent on a ring may be, for example, halogen, hydroxy, oxo, thioxo, thiol, amido, amino, carboxy, nitrilo, cyano, nitro, imino, oximo, hydrazino, alkoxy, alkenyl, alkynyl, aryl, aralkyl, aralkenyl, aralkynyl, cycloalkyl, cycloalkylalkyl, alkylcycloalkyl, heterocycloalkyl, heterocycyl, alkylheterocycyl, or any other useful substituent. A substituent may be water-soluble. Examples of water-soluble substituents include, but are not limited to, a pyridinium, an imidazolium, a quaternary ammonium group, a sulfonate, a phosphate, an alcohol, an amine, an imine, a nitrile, an amide, a thiol, a carboxylic acid, a polyether, an aldehyde, a boronic acid, and a boronic ester.

A linker may comprise a high solubility (e.g., a high aqueous solubility). A linker backbone may comprise an inherently high solubility. For example, a linker backbone may comprise a plurality of water-solubilizing amino acids. A linker may also comprise a water-soluble moiety that increases the linker's solubility. A linker may comprise a solubility of at least 0.01 g/L, at least 0.05 g/L, at least 0.1 g/L, at least 0.5 g/L, at least 1 g/L, at least 5 g/L, at least 10 g/L, at least 50 g/L, or at least 100 g/L The solubility may be an aqueous solubility. A linker may impart a high solubility to labeled reagent. Accordingly, a labeled reagent (e.g., a labeled nucleotide) consistent with the present disclosure may comprise a solubility of at least 0.01 g/L, at least 0.05 g/L, at least 0.1 g/L, at least 0.5 g/L, at least 1 g/L, at least 5 g/L, at least 10 g/L, at least 50 g/L, or at least 100 g/L. A labeled reagent comprising a linker can comprise a solubility that is approximately equal to that of the linker.

A linker can have any number of rings, including at least 1, 2, 3, 4, 5, 6, 7, 8, 9, 10, 11, 12, 13, 14, 15, 16, 17, 18, 19, 20, or more rings. The rings can share an edge in some cases (e.g., be components of a bicyclic ring system). In general, the ring portion of the linker can provide a degree of physical rigidity to the linker and/or can serve to physically separate the dye (e.g., fluorescent dye) on one end of the linker from the substrate to be labeled and/or from a second dye (e.g., fluorescent dye) associated with the substrate and/or associated with the linker. A ring can be a component of an amino acid (e.g., a non-proteinogenic amino acid, as described herein).

In some cases, a linker may be “fully rigid” (e.g., substantially inflexible). For example, ring systems of the linker may not be separated by any sp² or sp³ carbon atoms. In general, sp² and sp³ carbon atoms (e.g., between ring systems) provide the linker with a degree of physical flexibility. sp³ carbon atoms in particular can confer significant flexibility. Without limitation, flexibility can allow a polymerase to accept a substrate (e.g., a nucleotide or nucleotide analog) modified with the linker and the dye (e.g., fluorescent dye), or otherwise improve the performance of a labeled system. However, in a multiple dye system (e.g., a system comprising multiple fluorescent labeling reagents, such as a polynucleotide including two or more nucleotides coupled to two or more fluorescent labeling reagents), an overly flexible linker may defeat the feature of rigidity and allow two dyes (e.g., fluorescent dyes) to come into close association and be quenched. Accordingly, ring systems of a linker may be connected to each other by a limited number of sp³ bonds, such as by no more than two sp³ bonds (e.g., 0, 1, or 2 sp³ bonds). For example, at least two ring systems of a linker may be connected to each other by no more than two sp³ bonds (e.g., by 0, 1, or 2 sp³ bonds). For example, at least two ring systems of a linker may be connected to each other by a no more than two sp² bonds, such as by no more than 1 sp² bond. Ring systems of a linker may be connected to each other by a limited number of atoms, such as by no more than 2 atoms. For example, at least two ring systems of a linker may be connected to each other by no more than 2 atoms, such as by only 1 atom or by no atoms (e.g., directly connected).

The series of ring systems of a linker may comprise aromatic and/or aliphatic rings. At least two ring systems of a linker may be connected to each other directly without an intervening carbon atom. A linker may comprise at least one amino acid that may comprise a ring system. For example, a linker may comprise at least one non-proteinogenic amino acid (e.g., as described herein), such as a hydroxyproline.

Many applications of optical (e.g., fluorescent) labeling reagents (e.g., nucleic acid sequencing reactions) can be performed in aqueous solutions. In some cases, a linker that has too high of a proportion of carbon and hydrogen atoms and/or a lack of charged chemical groups can be insufficiently water-soluble to be useful in an aqueous solution. Therefore, the linkers described herein can have a water-soluble group or groups.

A linker may include a water-soluble group at any useful position. For example, a linker may comprise a water-soluble group at or near a point of attachment to a label (e.g., dye, as described herein). Alternatively or in addition to, a linker may comprise a water-soluble group at or near a point of attachment to a substrate (e.g., a protein or a nucleotide or nucleotide analog). Alternatively or in addition to, a linker may comprise a water-soluble group between points of attachment to a label (e.g., dye, as described herein) and a substrate (e.g., a protein or a nucleotide or nucleotide analog). One or more rings of a linker may comprise a water-soluble group. For example, each of the rings may comprise a water-soluble group, two or more rings may comprise a water-soluble group, only one of the rings may comprise a water-soluble group, or anywhere there between. A given ring may comprise one or more water-soluble moieties. For example, a ring of a linker may comprise two water-soluble moieties. The water-soluble group(s) can be a constituent part of the backbone of a ring of a linker or can be appended to a ring of a linker (e.g., as a substituent). Each water-soluble moiety of a linker may be different. Alternatively, one or more water-soluble moieties of a linker may be the same. For example, each water-soluble moiety of a linker may be the same. In some cases, the water-soluble group is positively charged. Examples of suitable water-soluble groups include, but are not limited to, a pyridinium, an imidazolium, a quaternary ammonium group, a sulfonate, a phosphate, an alcohol, an amine, an imine, a nitrile, an amide, a thiol, a carboxylic acid, a polyether, an aldehyde, and a boronic acid or boronic ester.

A water-soluble group can be any functional group that decreases (including making more negative) the log P of the optical (e.g., fluorescent) labeling reagent. Log P is the partition coefficient for a molecule between water and n-octanol. A greasy molecule is more likely to partition into octanol, giving a positive and large log P value. A formula for Log P can be represented as log P_{octanol/water}=log ([solute]_{octanol}/[solute]_{water}), where [solute]_{octanol} is the concentration of the solute (i.e., the labeling reagent) in octanol and [solute]_{water} is the concentration of the solute in water. Therefore, the more a compound partitions into water compared to octanol, the more negative the log P. Log P can be measured experimentally or predicted using software algorithms. The water-soluble group can have any suitable Log P value. In some cases, the Log P is less than about 2, less than about 1.5, less than about 1, less than about 0.5, less than about 0, less than about −0.5, less than about −1, less than about −1.5, less than about −2, or lower. In some cases, the Log P is between about 2.0 and about −2.0.

A linker may include one or more asymmetric (e.g., chiral) centers (e.g., as described herein). All stereochemical isomers of linkers are contemplated, including racemates and enantiomerically pure linkers.

A linker, and/or a substrate (e.g., protein or nucleotide or nucleotide analog) or dye to which it may be attached, may include one or more isotopic (e.g., radio) labels (e.g., as described herein). All isotopic variations of linkers are contemplated.

The structural features of a linker, including the number of rings, the rigidity of the linker, and the like, can combine to establish a functional distance between a dye (e.g., fluorescent) dye and a substrate (e.g., protein or nucleotide or nucleotide analog) that are linked by the linker. In some cases, the distance corresponds to the length (and/or the functional length) of the linker. In some cases, the functional length varies based on the temperature, solvent, pH, and/or salt concentration of the solution in which the length is measured or estimated. The functional length can be measured in a solution in which an optical (e.g., fluorescent) signal from the substrate is measured. The functional length may an average or ensemble value of a distribution of functional lengths (e.g., over rotational, vibrational, and translational motions) and may differ based on, e.g., temperature, solvent, pH, and/or salt concentrations. The functional length may be estimated (e.g., based on bond lengths and steric considerations, such as by use of a chemical drawing or modeling program) and/or measured (e.g., using molecular imaging and/or crystallographic techniques).

A linker can establish any suitable functional length between a dye (e.g., fluorescent dye) and a substrate (e.g., protein or nucleotide or nucleotide analog). In some cases, the functional length is at most about 500 nanometers (nm), at most about 200 nm, at most about 100 nm, at most about 75 nm, at most about 50 nm, at most about 40 nm, at most about 30 nm, at most about 20 nm, at most about 10 nm, at most about 5 nm, at most about 2 nm, at most about 1.0 nm, at most about 0.5 nm, at most about 0.3 nm, at most about 0.2 nm, or less. In some cases, the functional length is at least about 500 nanometers (nm), at least about 200 nm, at least about 100 nm, at least about 75 nm, at least about 50 nm, at least about 40 nm, at least about 30 nm, at least about 20 nm, at least about 10 nm, at least about 5 nm, at least about 2 nm, at least about 1.0 nm, at least about 0.5 nm, at least about 0.3 nm, at least about 0.2 nm, or less. In some instances, the functional length is at least about 0.2 nanometers (nm), at least about 0.3 nm, at least about 0.5 nm, at least about 1.0 nm, at least about 2 nm, at least about 5 nm, at least about 10 nm, at least about 20 nm, at least about 30 nm, at least about 40 nm, at least about 50 nm, at least about 75 nm, at least about 100 nm, at least about 200 nm, at least about 500 nm, or more. In some instances, the functional length is between about 0.5 nm and about 50 nm.

In some cases, the linker forms a straight and/or contiguous chain. In some instances, the linker is branched. The linker can be capable of forming a bond with a plurality of dyes (e.g., fluorescent dyes) and/or substrates (e.g., nucleotides and/or nucleotide analogs). In some instances, a linker comprises at most one dye per branch. In some instances, a linker comprises multiple dyes coupled to a single branch.

A linker may be a polymer having a regularly repeating unit. Alternatively, a linker may be a co-polymer without a regularly repeating unit. In some cases, the linker is not the result of a polymerization process. In general, a polymerization process can generate products having a variety of degrees of polymerization and molecular weights. In contrast, in some cases, the linkers described herein have a defined (i.e., known) molecular weight.

A linker may be constructed from one or more amino acids. For example, a linker may be constructed from two or more amino acids. A linker may comprise at least 5 amino acids. A linker may comprise at least 10 amino acids. A linker may comprise at least 15 amino acids. A linker may comprise at least 20 amino acids. A linker may comprise at least 25 amino acids. A linker may comprise at least 30 amino acids. A linker may comprise at least 5 non-proteinogenic amino acids. A linker may comprise at least 10 non-proteinogenic amino acids. A linker may comprise at least 15 non-proteinogenic amino acids. A linker may comprise at least 20 non-proteinogenic amino acids. A linker may comprise at least 25 non-proteinogenic amino acids. A linker may comprise at least 30 non-proteinogenic amino acids. A linker may comprise at least 5 prolines or proline analogues. A linker may comprise at least 10 prolines or proline analogues. A linker may comprise at least 15 prolines or proline analogues. A linker may comprise at least 20 prolines or proline analogues. A linker may comprise at least 25 prolines or proline analogues. A linker may comprise at least 30 prolines or proline analogues. A linker may comprise at least 5 contiguous amino acids. A linker may comprise at least 10 contiguous amino acids. A linker may comprise at least 15 contiguous amino acids. A linker may comprise at least 20 contiguous amino acids. A linker may comprise at least 25 contiguous amino acids. A linker may comprise at least 30 contiguous amino acids. A linker may comprise at least 5 contiguous prolines or proline analogues. A linker may comprise at least 10 contiguous prolines or proline analogues. A linker may comprise at least 15 contiguous prolines or proline analogues. A linker may comprise at least 20 contiguous prolines or proline analogues. A linker may comprise at least 25 contiguous prolines or proline analogues. A linker may comprise at least 30 contiguous prolines or proline analogues. A linker may comprise an oligopeptide comprising a glycine coupled to 5-20 consecutive prolines or proline analogs. A linker may comprise an oligopeptide comprising a glycine coupled to 10 consecutive prolines or proline analogs.

A plurality of amino acids in a linker may comprise a stable or semi-stable secondary structural feature, such as an alpha-helix. The secondary structural feature may comprise a helical structure. In some cases, the helical structure comprises a polyproline helix. The secondary structural feature (e.g., a polyproline helical structure) may increase the rigidity of a linker and may diminish dye-dye interactions (e.g., quenching or emission spectrum distortion) in systems with multiple dye-coupled linkers.

An amino acid may be a natural amino acid or a non-natural amino acid. An amino acid may be a proteinogenic amino acid or a non-proteinogenic amino acid. A “proteinogenic amino acid,” as used herein, generally refers to a genetically encoded amino acid that may be incorporated into a protein during translation. Proteinogenic amino acids include arginine, histidine, lysine, aspartic acid, glutamic acid, serine, threonine, asparagine, glutamine, cysteine, selenocysteine, glycine, proline, alanine, isoleucine, leucine, methionine, phenylalanine, tryptophan, tyrosine, valine, selenocysteine, and pyrrolysine. A “non-proteinogenic amino acid,” as used herein, is an amino acid that is not a proteinogenic amino acid. A non-proteinogenic amino acid may be a naturally occurring amino acid or a non-naturally occurring amino acid. Non-proteinogenic amino acids include amino acids that are not found in proteins and/or are not naturally encoded or found in the genetic code of an organism. Examples of non-proteinogenic amino acids include, but are not limited to, hydroxyproline, selenomethionine, hypusine, 2-aminoisobutyric acid, αγ-aminobutyric acid, omithine, citrulline, β-alanine (3-aminopropanoic acid), δ-aminolevulinic acid, 4-aminobenzoic acid, dehydroalanine, carboxyglutamic acid, pyroglutamic acid, norvaline, norleucine, alloisoleucine, t-leucine, pipecolic acid, allothreonine, homocysteine, homoserine, α-amino-n-heptanoic acid, α,β-diaminopropionic acid, α,γ-diaminobutyric acid, β-amino-n-butyric acid, β-aminoisobutyric acid, isovaline, sarcosine, N-ethyl glycine, N-propyl glycine, N-isopropyl glycine, N-methyl alanine, N-ethyl alanine, N-methyl β-alanine, N-ethyl β-alanine, isoserine, and α-hydroxy-γ-aminobutyric acid. Additional examples of non-proteinogenic amino acids include the non-natural amino acids described herein. A non-proteinogenic amino acid may comprise hydroxyproline or pyroglutamic acid. A non-proteinogenic amino acid may comprise hydroxyproline. A non-proteinogenic amino acid may comprise a ring structure. For example, a non-proteinogenic amino acid may be trans-4-aminomethylcyclohexane carboxylic acid or 4-hydrazinobenzoic acid. Such compounds may be FMOC-protected with FMOC (fluorenylmethyloxycarbonyl chloride) and utilized in solid-phase peptide synthesis. The structures of these compounds are shown below:

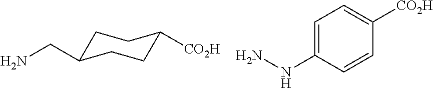 [SMILES: NCC1CCC(C(=O)O)CC1 ; NNc1ccc(C(=O)O)cc1]

Where a linker comprises multiple amino acids, such as multiple non-proteinogenic amino acids, an amine moiety adjacent to a ring moiety (e.g., the amine moiety in the hydrazine moiety) can function as a water-solubilizing group. To synthesize a water-soluble peptide, a hybrid linker can be made that comprises alternating non-water-soluble amino acids and water-soluble amino acids (e.g., hydroxyproline). Other moieties can be used to increase water-solubility. For example, linking amino acids with oxamate moieties can provide water-solubility through the additional hydrogen bonding without adding any sp³ linkages. The structure of the oxamate precursor 2-amino-2-oxoacetic acid is shown below:

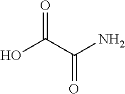

In some cases, a component (e.g., a monomer unit) of a linker may have an amino group, a carboxy group, and a water-solubilizing moiety. In some cases, a monomer may be deconstructed as two “half-monomers.” That is, by using two different units, one that contains two amino groups and another that contains two carboxy groups, an amino acid moiety can be constructed, which amino acid moiety may be a unit (e.g., a repeated unit) of a linker. One or both units may include one or more water solubilizing moieties. For example, at least one unit may include a water-soluble group (e.g., as described herein). For example, 2,5-diaminohydroquinone can be one half-monomer (A), and 2,5-dihydroxyterephthalic acid may be the other half-monomer (B). Such a scheme is shown below:

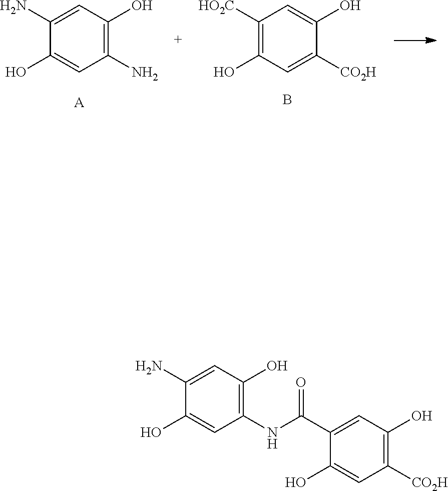

As shown above, A is a diamine and B is a diacid. Accordingly, non-proteinogenic (e.g., non-natural) amino acids may be constructed from diamines and diacids. An additional example of such a construction is shown below:

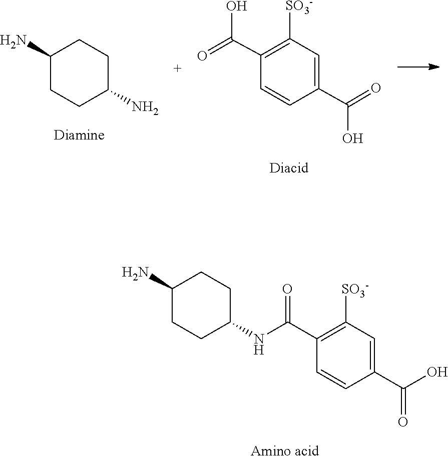

A polymer based on two half-monomers (e.g., as shown above) can be constructed via solid phase synthesis. Because the half-monomers can be homobifunctional in the linking moiety, in some cases no FMOC protection is required. For example, the dicarboxylic acid can be appended to the solid support, then an excess of the diamine added with appropriate coupling reagent (HBTU/HOBT/collidine). After washing away excess reagent, an excess of the dicarboxylic acid can be added with the coupling reagent. Side-products consisting of one molecule of the fluid phase reagent reacting with two solid-phase attached reagent can result in truncation of the synthesis. These side products can be separated from a product after cleavage from the support and purification by HPLC.

An advantage of the half-monomers approach can be increased flexibility in creating polymers. The diamine (A) can be replaced in a subsequent step by a different diamine (A′) to change the properties of the polymer, in a repeating or non-repeating manner. Such a scheme may facilitate construction of a polymer such as ABA′BABA′B.

Additional examples of half-monomers for use according to the schemes described above include 2,5-diaminopyridine and 2,5-dicarboxypyridine, both of which are shown below, as well as the other moieties shown below:

Diamines:

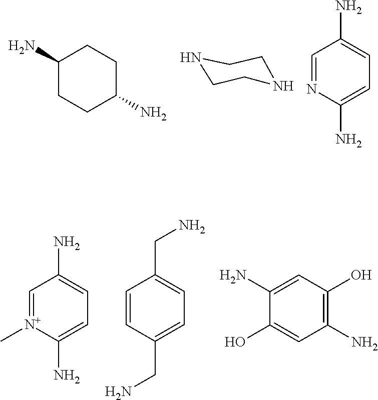

Dicarboxylic Acids:

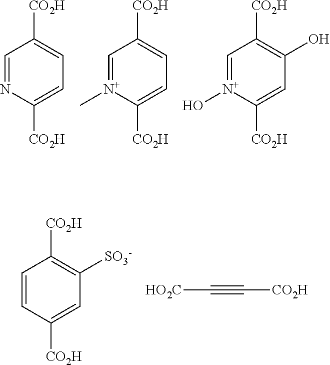

As described above, an amino acid (e.g., a non-proteinogenic amino acid that may be a non-natural amino acid) may be constructed from a diamine and a dicarboxylic acid. An amino acid (e.g., a non-proteinogenic amino acid that may be a non-natural amino acid) may also be constructed from an amino thiol and a thiol carboxylic acid. Examples of amino thiols and thiol carboxylic acids are shown below:

Amino Thiols:

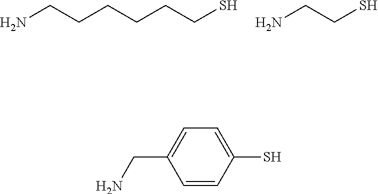

Thiol Carboxylic Acids:

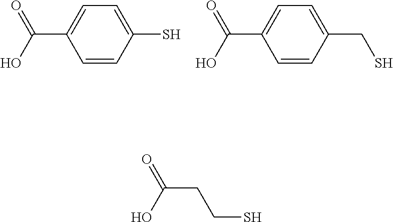 [SMILES: O=C(O)c1ccc(S)cc1 ; O=C(O)c1ccc(CS)cc1 ; O=C(O)CCS]

Examples of amino acids (e.g., non-natural amino acids) constructed from an amino thiol and a thiol carboxylic acid are shown below:

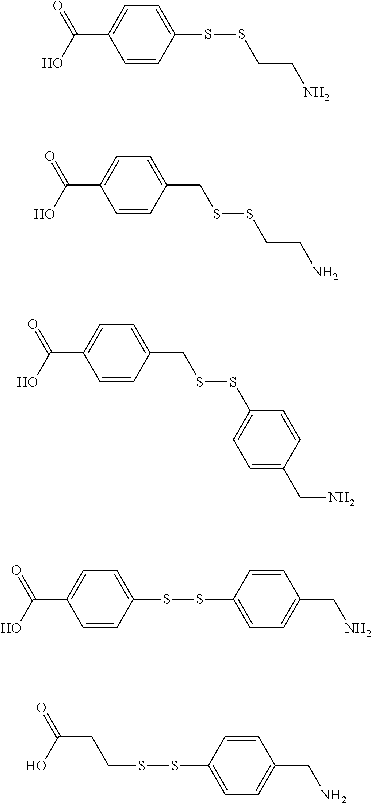 [SMILES: NCCSSc1ccc(C(=O)O)cc1 ; NCCSSCc1ccc(C(=O)O)cc1 ; NCc1ccc(SSCc2ccc(C(=O)O)cc2)cc1 ; NCc1ccc(SSc2ccc(C(=O)O)cc2)cc1 ; NCc1ccc(SSCCC(=O)O)cc1]

As shown above, amino acids constructed using an amino thiol and a thiol carboxylic acid may include a disulfide bond. As described elsewhere herein, a disulfide bond may be cleavable using a cleavage reagent (e.g., as described herein). Accordingly, an amino acid constructed from an amino thiol and a thiol carboxylic acid may serve as a cleavable portion of a linker. An amino acid constructed from an amino thiol and a carboxylic acid may be a component of a linker (e.g., as described herein) that may couple labeling moiety (e.g., a fluorescent dye) to a substrate (e.g., a nucleotide or nucleotide analog). The various structures allow different hydrophobicities for incorporation and may provide different “scar” moieties subsequent to interaction with a cleavage reagent (e.g., as described herein). Two or more amino acids, such as two or more amino acids constructed from an amino thiol and a thiol carboxylic acid, may be included in a linker. For example, two or more amino acids may be included in a linker and separated by no more than 2 sp³ carbon atoms, such as by no more than 2 sp² carbon atoms or by no more than 2 atoms. Where two or more amino acids formed of amino thiols and thiol carboxylic acids are connected to one another within a linker, cleavage may be more rapid as there will be multiple possible sites for cleavage. An example of a portion of a linker including such a component is shown below:

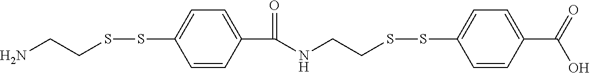 [SMILES: NCCSSc1ccc(C(=O)NCCSSc2ccc(C(=O)O)cc2)cc1]

As described above, two half-monomers may combine to provide an amino acid (e.g., a non-proteinogenic amino acid, such as a non-natural amino acid). Accordingly, a non-natural amino acid may include any known non-natural amino acid, as well as any non-natural amino acid that may be constructed as described herein.

Half-monomers such as those described herein can be constructed into polypeptide polymers. An example of a nucleotide constructed with two repeating units of an amino acid is shown below:

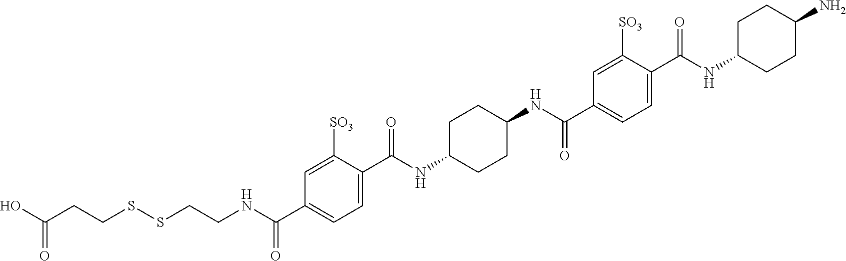

In some cases, before or after peptide coupling, the nitrogen in a nitrogen-containing ring can be quaternized to provide pyridinium moieties, thereby improving water-solubility of the final product. An example linker sequence generated in this manner is shown below:

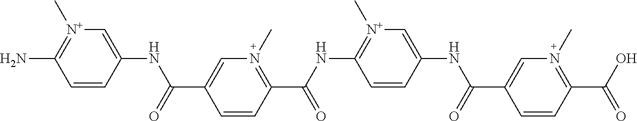

Water-solubilizing linkages that can work with the half-monomer method include, for example, those that have symmetrical functional groups, such as secondary amides, bishydrazides, and ureas. Examples of such moieties are shown below:

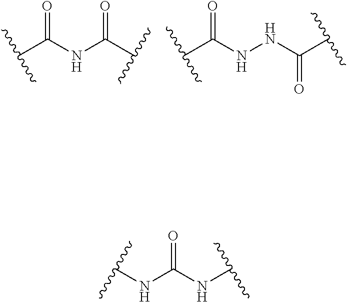 [SMILES: *C(=O)NC(*)=O ; *C(=O)NNC(*)=O ; *NC(=O)N*]

Amino acid linker subunits may be assembled into polymers by peptide synthesis methods. For example, a solid support method known as SPPS (Solid Phase Peptide Synthesis) or by liquid-phase synthesis may be used to assemble amino acids into a linker. SPPS methods can use a solid phase bead where the initial step is attachment of the C-terminal amino acid via its carboxylic acid moiety, leaving its free amine ready for coupling. Peptide synthesis can be initiated by flowing FMOC amine-protected monomers with peptide coupling reagents such as HBTU and an organic base. Excess reagent can be washed away, and the next monomer is then introduced. After one or more amino acids have been appended the final peptide can be cleaved from the beads and purified by HPLC. Liquid phase synthesis can use the same reagents (except the beads), but purification occurs after each step. The advantage of either stepwise polymerization process is that the resultant linkers can have a defined molecular weight that may be confirmed by mass spectrometry.

A linker may include one or more components. For example, a linker may include a first component that includes a polymeric region (e.g., that includes a repeating unit) and a second unit that does not include a polymeric region. The second component may include a cleavable component (e.g., as described herein). Examples of cleavable linkers include, but are not limited to, the structures E and B shown below:

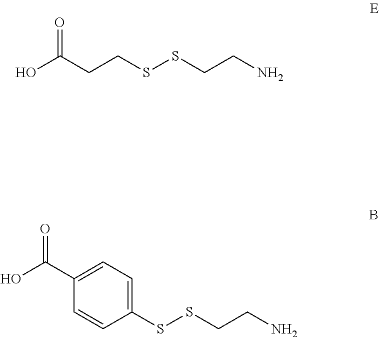

In the structures shown above, the disulfide moieties may be cleaved (e.g., as described herein) to provide thiol scars. The cleavable linkers may be attached to substrates upon reaction between a carboxyl moiety of the linker moiety and an amine moiety attached to a substrate (e.g., protein or nucleotide or nucleotide analog) to provide the substrate attached to the cleavable linker via an amide moiety. For example, the substrate may be a nucleotide or nucleotide analog including a propargylamino moiety, and a fluorescent labeling reagent comprising a dye and a linker described herein may be configured to associate with the substrate via the propargylamino moiety. Examples of such substrates are shown below:

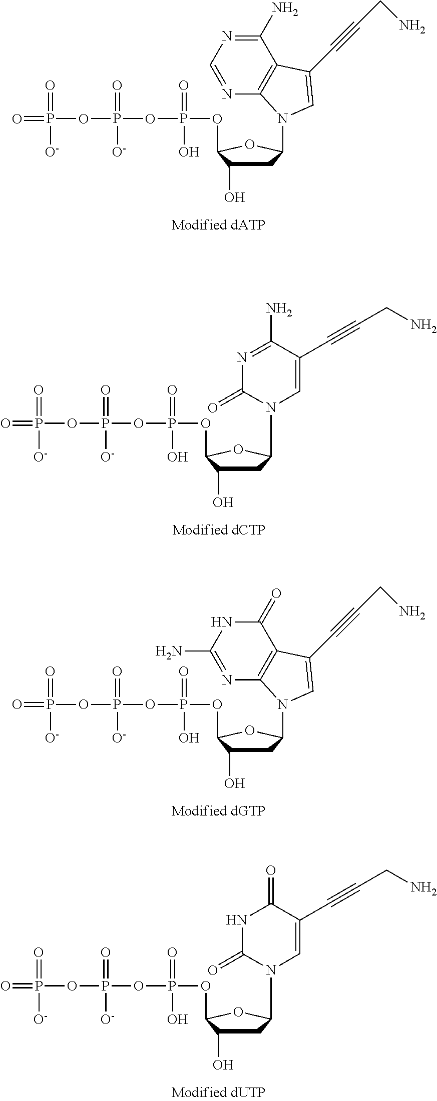

The first component of a linker including first and second components may include a repeating unit. For example, the linker may include a first component including one or more hydroxyproline moieties. An example of such a linker component is shown below:

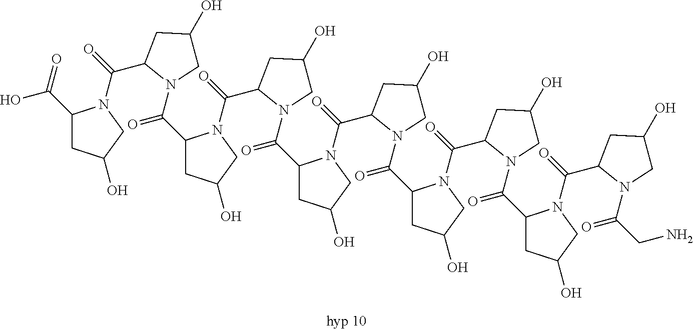

The linker shown above includes 10 hydroxyproline moieties and a glycine moiety and is referred to herein as “H” or “hyp10”. An alternate version of the linker above includes 20 hydroxyproline moieties and a glycine moiety and is referred to herein as “H₂₀” or “hyp20”. As described herein, all stereoisomers of hyp10 and hyp20, as well as combinations thereof, are contemplated. A linker component such as hyp10 can be linked to a cleavable linker via reaction between a free carboxyl moiety of the linker component and an amino moiety of a cleavable linker. A linker component such as hyp10 can be linked to a dye via the free amino moiety of the linker component. Examples of optical labeling reagent including a first linker component including a repeating unit (e.g., hyp10) and a second linker component including a cleavable linker are provided elsewhere herein. A linker may comprise a plurality of branches, wherein one or more of the branches comprises a hyp10 moiety, a hyp20 moiety, or any combination thereof.

A linker comprising an oligopeptide moiety (e.g., a hyp10 or hyp20 moiety) may provide a range of distances between the oligopeptide moiety and a second species, such as a substrate (e.g., a nucleotide), dye, or other species to which it is attached. A unit of measurement for this distance can be the number of atoms along the shortest path connected by contiguous bonds between the oligopeptide and second species (e.g., a substrate or dye), excluding atoms of the oligopeptide and the second species, and counting meta- and para-pathways across phenyl groups as two atoms. A linker may comprise at least 8 atoms, at least 10 atoms, at least 12 atoms, at least 15 atoms, at least 20 atoms, at least 25 atoms, at least 30 atoms, at least 40 atoms, or at least 50 atoms spacing the oligopeptide moiety and the second species. The linker may comprise at most 40 atoms, at most 30 atoms, at most 25 atoms, at most 20 atoms, at most 15 atoms, at most 10 atoms, at most 8 atoms, or at most 6 atoms spacing the oligopeptide moiety and the second species. A linker may be configured to provide an average distance of at least 1 nm, at least 1.2 nm, at least 1.5 nm, at least 2 nm, at least 3 nm, at least 4 nm, at least 5 nm, at least 6 nm, at least 8 nm, at least 10 nm, at least 12 nm, at least 15 nm, or at least 20 nm between the oligopeptide moiety and the second species. A linker may be configured to provide an average distance of at most 20 nm, at most 15 nm, at most 12 nm, at most 10 nm, at most 8 nm, at most 6 nm, at most 5 nm, at most 4 nm, at most 3 nm, at most 2 nm, at most 1.5 nm, at most 1.2 nm, or at most 1 nm between the oligopeptide moiety and the second species.

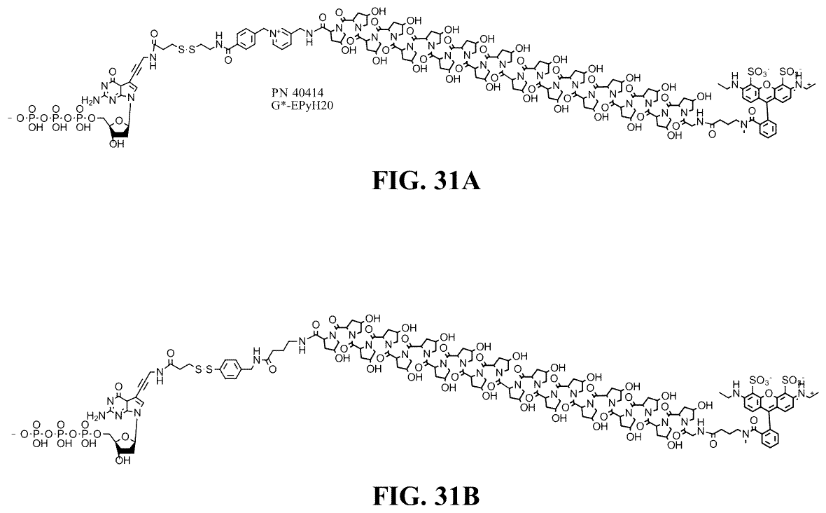

A linker may comprise one or more rings, such as a cycloalkyl group, a heterocycloalkyl group, an aromatic group, or a heteroaromatic group. Examples of linkers with aromatic and heteroaromatic groups are provided in FIGS. 31A and 31B, respectively.

Linkers may provide linkages between fluorescent moieties (e.g., dyes, as described herein) and substrates (e.g., proteins or nucleotides or nucleotide analogs). For example, an optical (e.g., fluorescent) labeling reagent may comprise an optical dye (e.g., fluorescent dye) attached to a linker (e.g., as described herein). Non-limiting examples of dyes (e.g., fluorescent dyes) include SYBR green, SYBR blue, DAPI, propidium iodine, Hoechst, SYBR gold, ethidium bromide, acridine, proflavine, acridine orange, acriflavine, fluorcoumanin, ellipticine, daunomycin, chloroquine, distamycin D, chromomycin, homidium, mithramycin, ruthenium polypyridyls, anthramycin, phenanthridines and acridines, propidium iodide, hexidium iodide, dihydroethidium, ethidium homodimer-1 and -2, ethidium monoazide, ACMA, Hoechst 33258, Hoechst 33342, Hoechst 34580, DAPI, acridine orange, 7-AAD, actinomycin D, LDS751, hydroxystilbamidine, SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO dyes (e.g., SYTO-40, -41, -42, -43, -44, and -45 (blue); SYTO-13, -16, -24, -21, -23, -12, -11, -20, -22, -15, -14, and -25 (green); SYTO-81, -80, -82, -83, -84, and -85 (orange); SYTO-64, -17, -59, -61, -62, -60, and -63 (red)), fluorescein, fluorescein isothiocyanate (FITC), tetramethyl rhodamine isothiocyanate (TRITC), rhodamine, tetramethyl rhodamine, R-phycoerythrin, Cy-2, Cy-3, Cy-3.5, Cy-5, Cy5.5, Cy-7, Texas Red, Phar-Red, allophycocyanin (APC), Sybr Green I, Sybr Green II, Sybr Gold, CellTracker Green, 7-AAD, ethidium homodimer I, ethidium homodimer II, ethidium homodimer III, ethidium bromide, umbelliferone, eosin, green fluorescent protein, erythrosin, coumarin, methyl coumarin, pyrene, malachite green, stilbene, lucifer yellow, cascade blue, dichlorotriazinylamine fluorescein, dansyl chloride, fluorescent lanthanide complexes such as those including europium and terbium, carboxy tetrachloro fluorescein, 5 and/or 6-carboxy fluorescein (FAM), VIC, 5- (or 6-) iodoacetamidofluorescein, 5-{[2(and 3)-5-(Acetylmercapto)-succinyl]amino} fluorescein (SAMSA-fluorescein), lissamine rhodamine B sulfonyl chloride, 5 and/or 6 carboxy rhodamine (ROX), 7-amino-methyl-coumarin, 7-Amino-4-methylcoumarin-3-acetic acid (AMCA), BODIPY fluorophores, 8-methoxypyrene-1,3,6-trisulfonic acid trisodium salt, 3,6-Disulfonate-4-amino-naphthalimide, phycobiliproteins, AlexaFluor dyes (e.g., AlexaFluor 350, 405, 430, 488, 532, 546, 555, 568, 594, 610, 633, 635, 647, 660, 680, 700, 750, and 790 dyes), DyLight dyes (e.g., DyLight 350, 405, 488, 550, 594, 633, 650, 680, 755, and 800 dyes), Black Hole Quencher Dyes (Biosearch Technologies) (e.g., BH1-0, BHQ-1, BHQ-3, and BHQ-10), QSY Dye fluorescent quenchers (from Molecular Probes/Invitrogen)(e.g., QSY7, QSY9, QSY21, and QSY35), Dabcyl, Dabsyl. Cy5Q, Cy7Q, Dark Cyanine dyes (GE Healthcare), Dy-Quenchers (Dyomics) (e.g., DYQ-660 and DYQ-661), ATTO fluorescent quenchers (ATTO-TEC GmbH) (e.g., ATTO 540Q, 580Q, 612Q, 532, and 633), and other fluorophores and quenchers (e.g., as described herein). In some cases, the label may be a type that does not self-quench or exhibit proximity quenching. Non-limiting examples of a label type that does not self-quench or exhibit proximity quenching include Bimane derivatives such as Monobromobimane. Additional dyes included in structures provided herein may also be utilized in combination with any of the linkers provided herein, and with any substrate described herein, regardless of the context of their disclosure.

A linker may comprise a cleavable group. A linker coupled to a detectable moiety (e.g., an optically detectable labeled reagent comprising a fluorescent dye) can comprise a cleavable group or moiety that is capable of being cleaved to separate the cleavable group from the substrate with which the optical labeled reagent is associated. The linker may be coupled to a substrate. In some cases, cleavage of the cleavable group or moiety may decouple the detectable moiety and the substrate. All or a portion of the linker may be part of the cleavable group. In some cases, cleaving a cleavable group may leave a scar group associated with substrate. The cleavable group can be, for example, an azidomethyl group capable of being cleaved by an agent such as tris(2-carboxyethyl)phosphine (TCEP), dithiothreitol (DTT), or tetrahydropyranyl (THP) to leave a hydroxyl scar group. The cleavable group can be, for example, a disulfide bond capable of being cleaved by an agent such as TCEP, DTT or THP to leave a thiol scar group. The cleavable group can be, for example, a hydrocarbyldithiomethyl group capable of being cleaved by an agent such as TCEP, DTT or THP to leave a hydroxyl scar group. The cleavable group may comprise a photocleavable moiety. For example, the cleavable group can be, for example, a 2-nitrobenzyloxy group capable of being cleaved by ultraviolet (UV) light to leave a hydroxyl scar group. A linker or a labeled reagent comprising the linker may be stable in the absence of an agent, light (e.g., ultraviolet light), or condition (e.g., a particular pH range) capable of cleaving a cleavable linker. For example, a linker comprising a cleavable disulfide group may be stable in the absence of a reducing agent. A linker or a labeling reagent comprising the linker may comprise a half-life of at least 1 day, at least 2 days, at least 3 days, at least 4 days, at least 5 days, at least 7 days, at least 10 days, at least 2 weeks, at least 3 weeks, at least 4 weeks, at least 5 weeks, at least 6 weeks, at least 8 weeks, at least 10 weeks, at least 12 weeks, at least 15 weeks, at least 18 weeks, at least 24 weeks, at least 36 weeks, at least 45 weeks, at least 1 year, at least 1.5 years, at least 2 years, at least 3 years, at least 5 years, or at least 10 years. The half-life may pertain to a solubilized (e.g., dissolved in an aqueous solution) or dried form of the linker or the labeling reagent, and may pertain to a temperature of less than 0° C., about 0° C., about 5° C., about 10° C., about 15° C., about 20° C., about 25° C., about 30° C., about 40° C., about 50° C., about 60° C., about 70° C., about 80° C., or above 80° C. A long half-life may confer stability and repeatability to measurements performed using the linker or the labeling reagent.

An optical (e.g., fluorescent) labeling reagent may be configured to associate with a substrate such as a nucleotide or nucleotide analog (e.g., as described herein). Alternatively or additionally, an optical (e.g., fluorescent) labeling reagent may be configured to associate with a substrate such as a protein, cell, lipid, or antibody. For example, the optical labeling reagent may be configured to associate with a protein. A protein substrate may be any protein, and may include any useful modification, mutation, or label, including any isotopic label. For example, a protein may be an antibody such as a monoclonal antibody. A protein associated with one or more optical (e.g., fluorescent) labeling reagents (e.g., as described herein) may be, for example, an antibody (e.g., a monoclonal antibody) useful for labeling a cell, which labeled cell may be analyzed and sorted using flow cytometry.

An optical (e.g., fluorescent) labeling reagent (e.g., as described herein) can decrease quenching (e.g., between dyes coupled to nucleotides or nucleotide analogs incorporated into a growing nucleic acid strand, such as during nucleic acid sequencing). For example, an optical (e.g., fluorescent) signal emitted by a substrate (e.g., a nucleotide or nucleotide analog that may be incorporated into a growing nucleic acid strand) can be proportional to the number of optical (e.g., fluorescent) labels associated with the substrate (e.g., to the number of optical labels incorporated adjacent or in proximity to the substrate). For example, multiple optical labeling reagents including substrates of the same or different types (e.g., nucleotides or nucleotide analogs of a same or different type) may be incorporated in proximity to one another in a growing nucleic acid strand (e.g., during nucleic acid sequencing). In such a system, signal emitted by the collective substrates may be approximately proportional (e.g., linearly proportional) to the number of dye-labeled substrates incorporated. In other words, quenching may not significantly impact the signal emitted. This may be observable in a system in which 100% labeling fractions are used. Where less than 100% of substrates are labeled (e.g., less than 100% of nucleotides in a nucleotide flow are labeled), an optical (e.g., fluorescent) signal emitted by substrates (e.g., nucleotides or nucleotide analogs) incorporated into a plurality of growing nucleic acid strands (e.g., a plurality of growing nucleic acid strands coupled to sequencing templates coupled to a support, as described herein) may be proportional to the length of a homopolymer region of the growing nucleic acid strands. Similarly, where less than 100% of substrates are labeled (e.g., less than 100% of nucleotides in each of successive nucleotide flows are labeled), an optical (e.g., fluorescent) signal emitted by substrates (e.g., nucleotides or nucleotide analogs) incorporated into a plurality of growing nucleic acid strands (e.g., a plurality of growing nucleic acid strands coupled to sequencing templates coupled to a support, as described herein) may be proportional to the length of a heteropolymeric and/or homopolymer region of the growing nucleic acid strands. In some such cases, the intensity of a measured optical (e.g., fluorescent) signal may be linearly proportional to the length of a heteropolymeric and/or homopolymeric region into which substrates have incorporated. For example, a measured optical (e.g., fluorescent) signal may be linearly proportional with a slope of approximately 1.0 when optical (e.g., fluorescent) signal is plotted against the length in substrates of a heteropolymeric and/or homopolymeric region into which substrates have incorporated.

An optical (e.g., fluorescent) labeling reagent (e.g., as described herein) can decrease quenching in a protein system. When labeling proteins, quenching may start to happen at a fluorophore to protein ratio (F/P) of around 3. Using optical labeling reagents provided herein, higher F/P ratios, and thus brighter reagents, may be obtained. This may be useful for analyzing proteins (e.g., using imaging) and/or for analyzing cells labeled with proteins (e.g., antibodies) associated with one or more optical (e.g., fluorescent) labeling reagents.

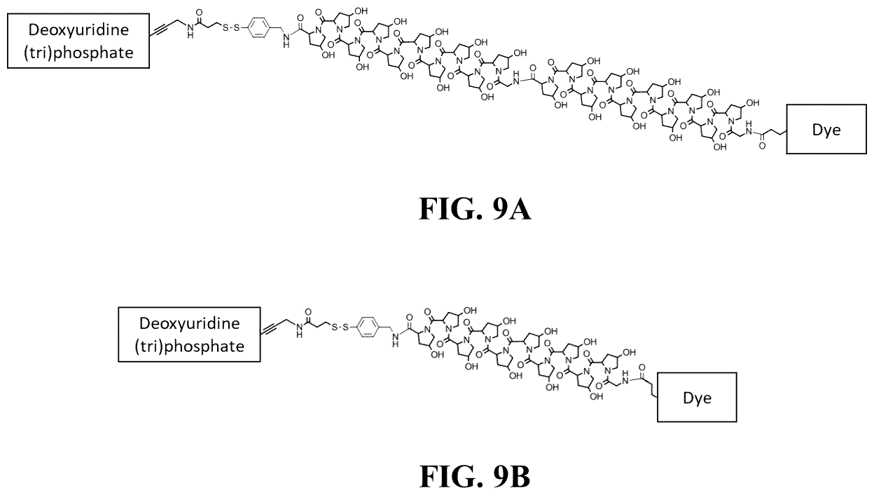

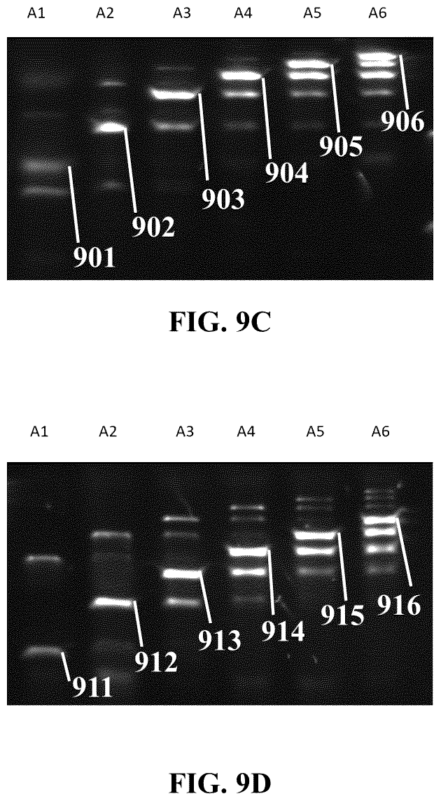

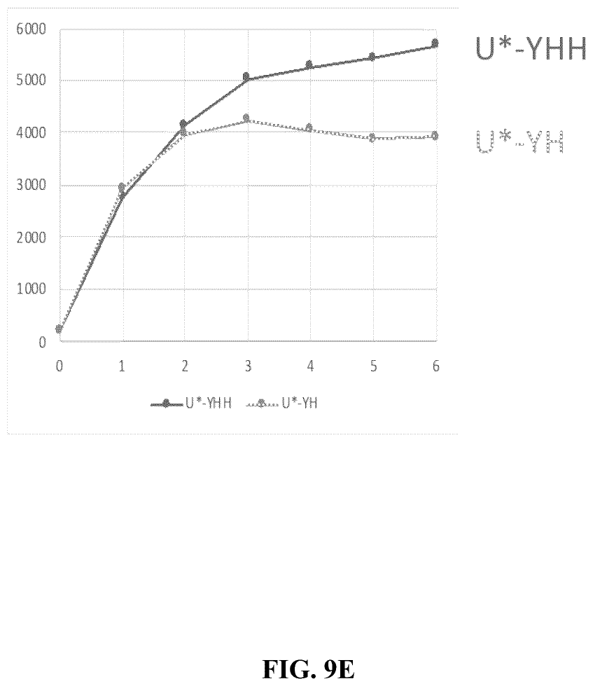

Examples of the linkers described herein are found, e.g., in FIGS. 9A, 9B, 10A, 10B, 12A, 14A, 14B, 17A, and 17B. In some cases, the linker comprises a group which confers water solubility to the labeling reagent. Additional examples are included elsewhere herein, including in the Examples below.

In as aspect, the present disclosure provides an oligonucleotide molecule comprising a fluorescent labeling reagent or derivative thereof (e.g., as described herein). The oligonucleotide molecule may comprise one or more additional fluorescent labeling reagents of a same type (e.g., comprising linkers having the same chemical structure, dyes comprising the same chemical structure, and/or associated with substrates (e.g., nucleotides) of a same type). The fluorescent labeling reagent and one or more additional fluorescent labeling reagents of the oligonucleotide molecule may be associated with nucleotides. For example, the fluorescent labeling reagents may be connected to nucleobases of nucleotides of the oligonucleotide molecule. A fluorescent labeling reagent and one or more additional fluorescent labeling reagent may be connected to adjacent nucleotides of the oligonucleotide molecule. Alternatively or in addition to, the fluorescent labeling reagent and the one or more additional fluorescent labeling reagents may be connected to nucleotides of the oligonucleotide molecule that are separated by one or more nucleotides that are not connected to fluorescent labeling reagents. The oligonucleotide molecule may be a single-stranded molecule. Alternatively, the oligonucleotide molecule may be a double-stranded or partially double-stranded molecule. A double-stranded or partially double-stranded molecule may comprise fluorescent labeling reagents associated with a single strand or both strands. The oligonucleotide molecule may be a deoxyribonucleic acid molecule. The oligonucleotide molecule may a ribonucleic acid molecule. The oligonucleotide molecule may be generated and/or modified via a nucleic acid sequencing process (e.g., as described herein).

The linker of the fluorescent labeling reagent may comprise a cleavable group that is configured to be cleaved to separate the fluorescent dye of the fluorescent labeling reagent from a substrate (e.g., nucleotide) with which it is associated. For example, the linker may comprise a cleavable group comprising an azidomethyl group, a disulfide bond, a hydrocarbyldithiomethyl group, or a 2-nitrobenzyloxy group. The cleavable group may be configured to be cleaved by application of one or more members of the group consisting of tris(2-carboxyethyl)phosphine (TCEP), dithiothreitol (DTT), tetrahydropyranyl (THP), ultraviolet (UV) light, and a combination thereof. The oligonucleotide molecule comprising a fluorescent labeling reagent may be configured to emit a fluorescent signal (e.g., upon excitation at an appropriate range of energy, as described herein).

In another aspect, the present disclosure provides a kit comprising a plurality of linkers (e.g., as described herein). A linker of the plurality of linkers may comprise (i) one or more water soluble groups and (ii) two or more ring systems. At least two of the two or more ring systems may be connected to each other by no more than two sp³ carbon atoms. For example, at least two of the two or more ring systems may be connected to each other by an sp² carbon atom. At least two of the two or more ring systems may be connected to each other by no more than two atoms. The linker may comprise a non-proteinogenic amino acid (e.g., as described herein) comprising a ring system of the two or more ring systems. For example, the linker may comprise a hydroxyproline or an amino acid constructed from, e.g., a diamine and a dicarboxylic acid or an amino thiol and a thiol carboxylic acid. The linker may be connected to a fluorescent dye (e.g., as described herein) and/or associated with a substrate. For example, the linker may be connected to a fluorescent dye and coupled to a substrate selected from a nucleotide, a protein, a lipid, a cell, and an antibody. For example, the linker may be connected to a fluorescent dye and a nucleotide.

The linker may comprise a plurality of amino acids, such as a plurality of non-proteinogenic (e.g., non-natural) amino acids. For example, the linker may comprise a plurality of hydroxyprolines (e.g., a hyp10 moiety). At least one water-soluble group of the one or more water-soluble groups may be appended to a ring structure of the two or more ring systems. The one or more water soluble groups may be selected from the group consisting of a pyridinium, an imidazolium, a quaternary ammonium group, a sulfonate, a phosphate, an alcohol, an amine, an imine, a nitrile, an amide, a thiol, a carboxylic acid, a polyether, an aldehyde, a boronic acid, and a boronic ester. The linker may comprise a cleavable group that is configured to be cleaved to separate a first portion of the linker from a second portion of the linker. The cleavable group may be selected from the group consisting of an azidomethyl group, a disulfide bond, a hydrocarbyldithiomethyl group, and a 2-nitrobenzyloxy group. The cleavable group may be cleavable by application of one or more members of the group consisting of tris(2-carboxyethyl)phosphine (TCEP), dithiothreitol (DTT), tetrahydropyranyl (THP), ultraviolet (UV) light, and a combination thereof. The linker may comprise a moiety selected from the group consisting of

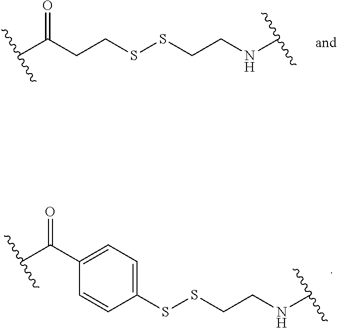 [SMILES: *NCCSSCCC(*)=O ; *NCCSSc1ccc(C(*)=O)cc1]

These moieties both comprise disulfide groups and so may be considered cleavable groups.

The plurality of linkers of the kit may comprise a first linker associated with a first substrate (e.g., a first nucleotide) and a second linker associated with a second substrate (e.g., a second nucleotide). The first substrate and the second substrate may be of different types (e.g., different canonical nucleotides). The first substrate and the second substrate may be nucleotides comprising nucleobases of different types (e.g., A, C, G, U, and T). The first linker and the second linker may comprise the same chemical structure. Similarly, the first linker may be connected to a first fluorescent dye and the second linker may be connected to a second fluorescent dye. The first fluorescent dye and the second fluorescent dye may be of different types. For example, the first and second fluorescent dyes may fluoresce at different wavelengths and/or have different maximum excitation wavelengths. The first and second fluorescent dyes may fluoresce at similar wavelengths and/or have similar maximum excitation wavelengths regardless of whether they share the same chemical structure.

The plurality of linkers of the kit may further comprise a third linker associated with a third substrate and a fourth linker associated with a fourth substrate. The first substrate, the second substrate, the third substrate, and the fourth substrate may be of different types. For example, the first substrate, the second substrate, the third substrate, and the fourth substrate may be nucleotides comprising nucleobases of different types (e.g., A, C, G, and U/T). The first linker and the third linker may comprise different chemical structures. The first and third linker may comprise a same chemical group, such as a same cleavable group (e.g., as described herein). For example, the first linker and the third linker may each comprise a moiety comprising a disulfide bond. Similarly, the first linker and the fourth linker may comprise different chemical structures. The first and fourth linker may comprise a same chemical group, such as a same cleavable group (e.g., as described herein). For example, the first linker and the fourth linker may each comprise a moiety comprising a disulfide bond.

In an example, the first linker comprises a hyp10 moiety and a first cleavable moiety, the second linker comprises a hyp10 moiety and a second cleavable moiety, the third linker comprises a third cleavable moiety and does not comprise a hyp10 moiety, and the fourth linker comprises a fourth cleavable moiety and does not comprise a hyp10 moiety. The second cleavable moiety may have a chemical structure that is different than the first cleavable moiety. Alternatively, the second cleavable moiety and the first cleavable moiety may have the same chemical structures. The third cleavable moiety and the fourth cleavable moiety may have the same chemical structure. Alternatively, the third cleavable moiety and the fourth cleavable moiety may have different chemical structures. In an example, the first linker and the second linker each have a first chemical structure and the third linker and the fourth linker each have a second chemical structure, which second structure is different than the first chemical structure. In another example, the first linker, the second linker, the third linker, and the fourth linker all have the same chemical structure. In another example, the first linker, the second linker, the third linker, and the fourth linker all have different chemical structures.

### Scarred Substrates

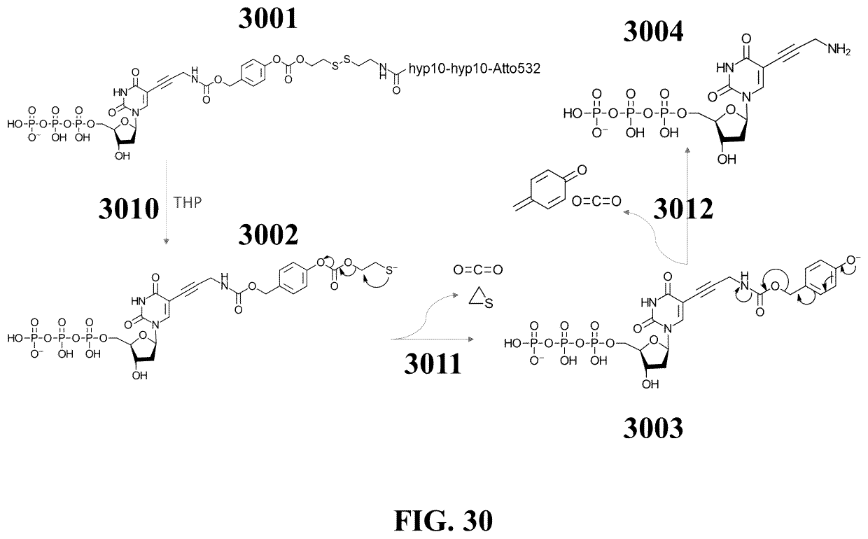

Following cleavage, a portion of a linker may remain bound to a substrate. Such a residual portion of a linker may be referred to as a scar or as a cleaved linker. In an example, prior to labeling, a substrate may be functionalized to include a functional handle that is subsequently used to couple the substrate to a linker. Following cleavage and a post-cleavage reaction (e.g., an immolation reaction), such a functional handle may be part of a scar or a cleaved linker. For example, the propargyl amine of the post-cleavage reaction product 3004 of FIG. 30 may be referred to as a scar, even if the labeled substrate synthesis for generating the labeled dUTP utilized propargyl amine functionalized dUTP. A scar of a biomolecule may comprise a portion of the biomolecule not typically associated with a canonical biomolecule of the same type.

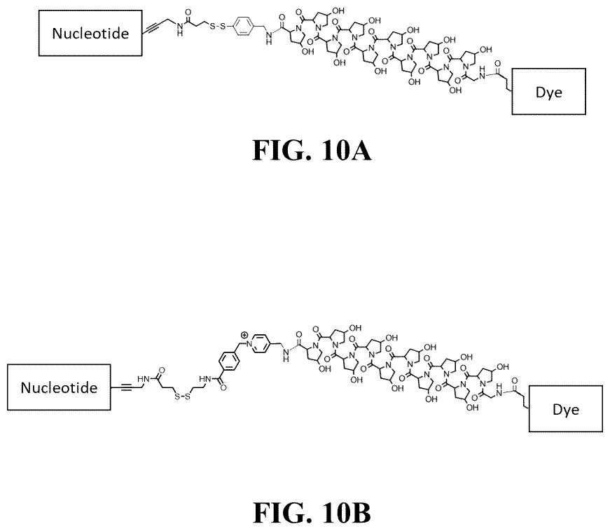

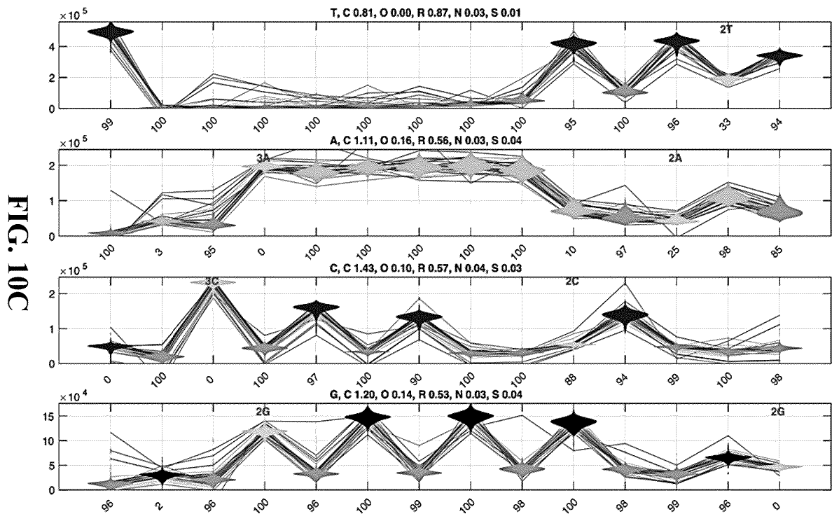

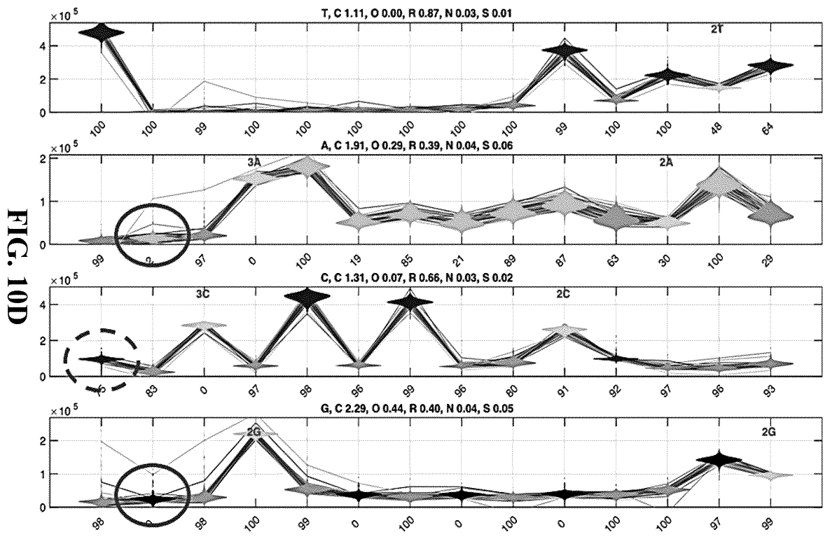

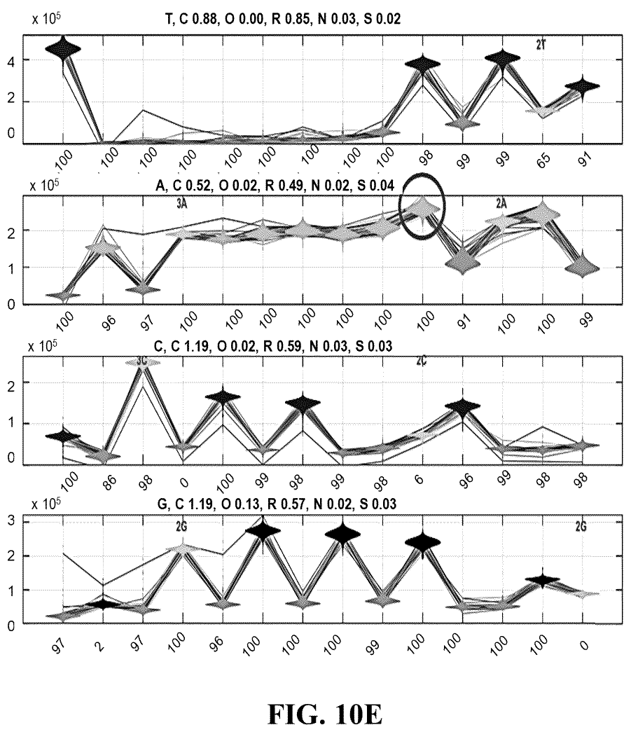

In some cases, a scar may alter a property of a substrate. For example, a scarred (i.e., scar-containing) nucleotide within a nucleic acid may inhibit further nucleotide incorporations into the nucleic acid. The scarred nucleotide may inhibit nucleotide incorporations at an immediately adjacent open position or may inhibit multiple subsequent nucleotide additions. For example, the sequencing data in FIG. 10C suggest that certain scars may inhibit nucleotide incorporations at any position within three nucleotides of the scar. In some cases, a scar may affect an optical property of a substrate. For example, a scar may quench fluorescence activity. In some cases, a scar may be reactive toward another species in a system, which may alter the performance of a system. For example, a nucleotide-bound scar may comprise a reactivity toward lysines, and thereby inhibit polymerase activity in a system. Thus, in some cases, cleavable linker performance can be enhanced by optimizing a scar's structure and properties. A scar may be stable upon cleavage.

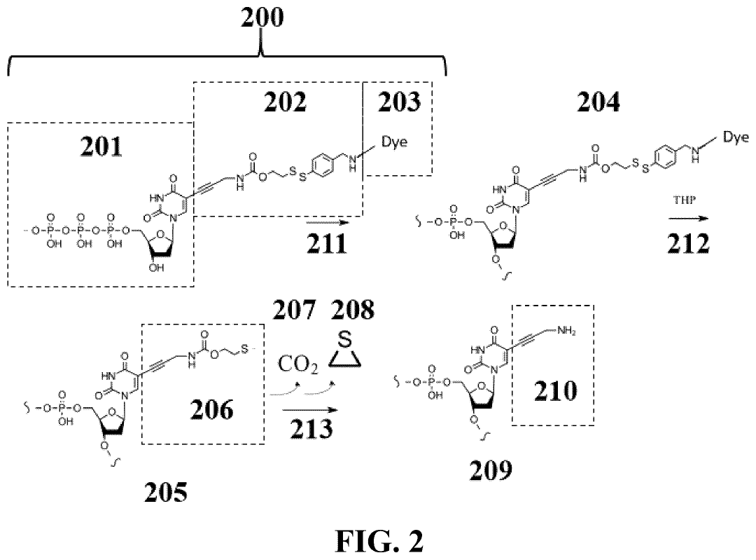

A scar may also be reactive. The scar's reactivity may be an intramolecular reactivity. In such cases, a scar may undergo a post-cleavage reaction to form a structure distinct from the initial scar formed upon cleavage. Such a post-cleavage reaction may be referred to as “immolation,” and scars which have undergone immolation may be referred to as “immolated scars.” A linker may spontaneously immolate (i.e., undergo immolation) upon cleavage, or may form a first scar that is stable until it is contacted with a reagent or a specific condition (e.g., a specific pH range). Immolation may change a physical or chemical property of the scar group, and further may diminish its size. An example of a post-cleavage reaction (e.g., an immolation reaction) is provided in FIG. 2. A labeling reagent 200 may comprise a substrate (e.g., a substrate for nucleic acid polymerization such as a nucleoside triphosphate) 201 coupled (e.g., covalently or non-covalently attached) to a cleavable linker 202. The cleavable linker may be coupled to a detectable moiety 203, such as a dye. The labeling reagent may be subjected to a chemical or enzymatic process 211, such as incorporation into a nucleic acid. The chemical or enzymatic process may chemically alter the labeling reagent 204, for example by forming a bond between the labeling reagent and a nucleic acid while removing a pyrophosphate group. The cleavable linker may be cleaved 212, yielding a truncated labeling reagent 205. The cleavage 212 may result in the loss of a detectable moiety from the labeling reagent. The cleavage may also generate a scar 206 on the labeling reagent. The scar may comprise a portion of the cleavable linker.

In some cases, a scar may be stable, such that the scar does not further react after forming. In some cases, a scar may undergo an immolation reaction 213, resulting in the loss of one or more moieties from the labeling reagent 207 and 208, and yielding an immolated labeling reagent 209. The immolated labeling reagent may comprise a new scar 210 or may comprise no scar at all.

A post-cleavage reaction (e.g., an immolation reaction) may comprise an intramolecular step (e.g., a decomposition step), an intermolecular step, such as a hydrolysis step, or any combination thereof. A post-cleavage reaction may be purely intramolecular. An intramolecular step may comprise nucleophilic substitution. For example, a thiolate scar may react with a carbonyl on the scarred substrate, which may result in scarred substrate cleavage at the carbonyl carbon. A post-cleavage reaction may solely comprise intermolecular steps. An intermolecular step may comprise a reaction between the scarred substrate and a solvent molecule (e.g., water, methanol, or ethanol), a proton donor (e.g., the scar is protonated during a step of the post-cleavage reaction), a proton acceptor (e.g., the scar is deprotonated during a step of the post-cleavage reaction), an oxidizing agent (e.g., the scar is oxidized during a step of the post-cleavage reaction), a reducing agent (e.g., the scar is reduced during a step of the post-cleavage reaction), an ion, a nucleophile, an electrophile, or any combination thereof. A post-cleavage reaction may be photoactivated (e.g., require or be accelerated by absorption of a photon). A post-cleavage reaction may be catalyzed.

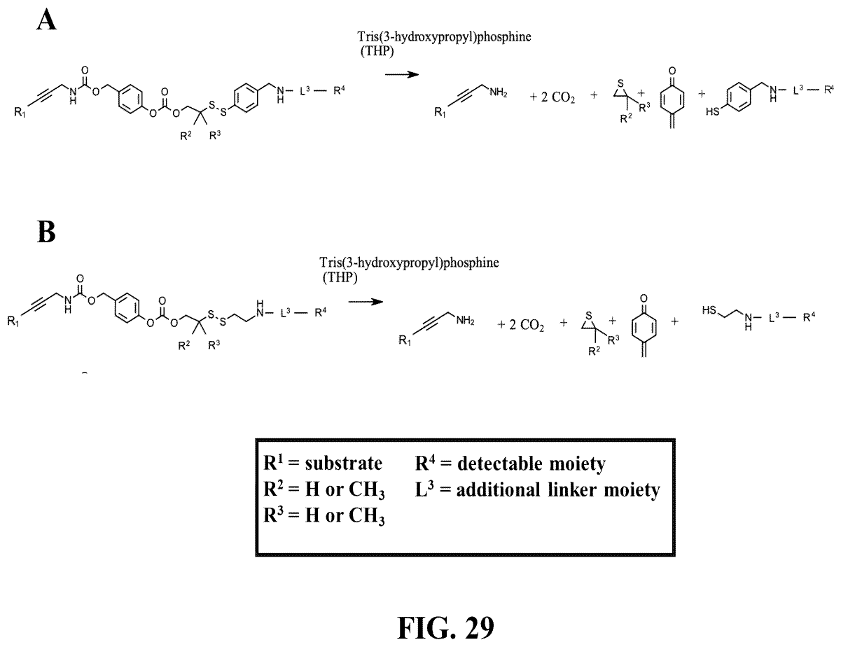

A post-cleavage reaction may comprise a decomposition step which liberates a moiety from a scarred substrate. For example, a post-cleavage reaction may comprise a CO₂ or thiirane liberating step, such as the thiirane decomposition of 213 in FIG. 2. A post cleavage reaction may also liberate a dearomatized moiety, such as a cyclohexadiene, a cyclohexadienone, a methylene cyclohexadienone, a cyclohexadiene thione, etc. Examples of dearomatizing decomposition reactions are provided in FIG. 29 panels A and B, which outline cleavage steps followed by post-cleavage decomposition reactions to liberate 4-methylenecyclohexadienone. A post-cleavage reaction may liberate at least 1, at least 2, at least 3, at least 4, at least 5, or at least moieties from the scarred substrate. The liberated molecules may limit post-cleavage reaction reversibility and may provide an entropic driving force to accelerate the post-cleavage reaction.

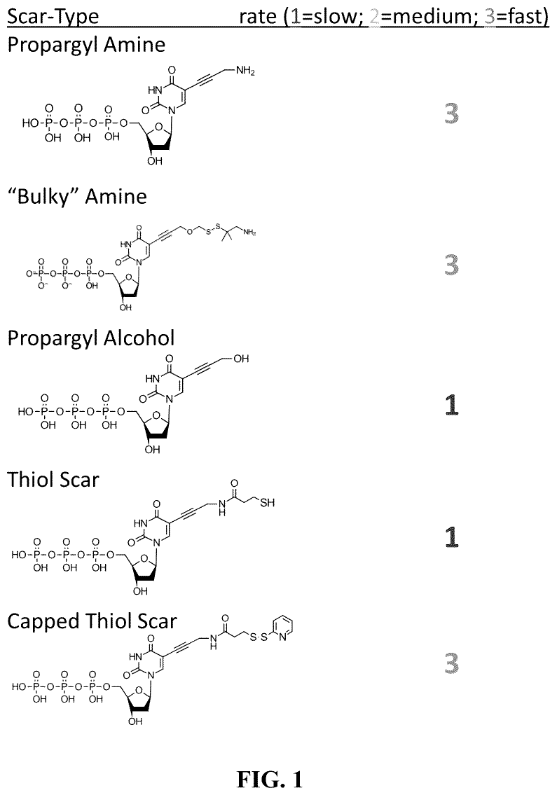

An immolated scar may comprise different properties than the post-cleavage scar from which it formed, which may make the immolated scar more favorable for a particular assay. In some cases, an immolated scar may inhibit an enzymatic activity (e.g., polymerase activity) less than the post-cleavage scar from which it formed. This concept is illustrated in FIG. 1, which compares BST type polymerase mediated nucleotide incorporation rates at positions directly adjacent to nucleotides comprising a variety of scars. As can be seen from the figure, thiol and propargyl alcohol scars (which can form directly from linker cleavage) can inhibit polymerization more than propargyl amine and primary aliphatic amine scars (which may be formed through scar immolation). In some cases, a less acidic scar (e.g., a scar comprising a higher pH) may inhibit an enzymatic activity less than a more acidic scar. In some cases, a smaller (e.g., lower mass, volume, or length) scar may inhibit an enzymatic activity less than a more acidic scar.

A linker may comprise a cleavable moiety that is configured to immolate following cleavage. A linker may comprise a first moiety that is configured to undergo cleavage (e.g., chemical or photocleavage) and a second moiety configured to immolate following cleavage of the first moiety. Cleavage of the first moiety may generate a reactive group that participates in immolation of the second moiety. For example, the first moiety may comprise a disulfide that forms a thiol upon cleavage, which itself reacts with an electrophilic center (e.g., a carbonyl) of the second moiety during immolation. Cleavage of the first moiety may generate a scar configured for immolation. In some cases, the immolation of the scar of the first moiety may promote immolation of the second moiety. For example, the first moiety may comprise a disulfide that forms a thiol upon cleavage, which may be configured to oxidatively eliminate as a thiirane or thietane, thereby promoting decarboxylation of a carbamate within the second moiety.

The post-cleavage reaction (e.g., generation of an immolated scar following cleavage) may occur at a range of rates. A linker may be configured to undergo a cleavage reaction followed by a slower post-cleavage reaction, and thereby to build up a pre-immolation scar intermediate. A linker may be configured for a cleavage reaction followed by a faster post-cleavage reaction. The rates of the cleavage and post-cleavage reactions may respond differently to changes in conditions. For example, an increase in pH may increase the rate of a cleavage reaction and decrease the rate of a post-cleavage reaction. The post-cleavage (e.g., immolation) reaction may comprise a first order rate constant of at least 0.01 hour⁻¹, at least 0.02 hour⁻¹, at least 0.03 hour⁻¹, at least 0.04 hour⁻¹, at least 0.05 hour⁻¹, at least 0.06 hour⁻¹, at least 0.08 hour⁻¹, at least 0.1 hour⁻¹, at least 0.12 hour⁻¹, at least 0.15 hour⁻¹, at least 0.2 hour⁻¹, at least 0.25 hour⁻¹, at least 0.3 hour⁻¹, at least 0.4 hour⁻¹, at least 0.5 hour⁻¹, at least 0.6 hour⁻¹, at least 0.7 hour⁻¹, at least 0.8 hour⁻¹, at least 0.9 hour⁻¹, at least 1 hour⁻¹, at least 2 hour⁻¹, at least 3 hour⁻¹, at least 4 hour⁻¹, at least 5 hour⁻¹, at least 6 hour⁻¹, at least 0.12 minute⁻¹, at least 0.15 minute⁻¹, at least 0.2 minute⁻¹, at least 0.25 minute⁻¹, at least 0.3 minute⁻¹, at least 0.4 minute⁻¹, at least 0.5 minute⁻¹, at least 0.6 minute⁻¹, at least 0.7 minute⁻¹, at least 0.8 minute⁻¹, at least 0.9 minute⁻¹, at least 1 minute⁻¹, at least 2 minute⁻¹, at least 3 minute⁻¹, at least 4 minute⁻¹, at least 5 minute⁻¹, at least 6 minute⁻¹, at least 0.12 second⁻¹, at least 0.15 second⁻¹, at least 0.2 second⁻¹, at least 0.3 second⁻¹, at least 0.4 second⁻¹, at least 0.5 second⁻¹, at least 0.6 second⁻¹, at least 0.7 second⁻¹, at least 0.8 second⁻¹, at least 0.9 second⁻¹, at least 1 second⁻¹, at least 1.5 second⁻¹, at least 2 second⁻¹, at least 3 second⁻¹, at least 5 second⁻¹, at least 8 second⁻¹, at least 10 second⁻¹, at least 12 second⁻¹, at least 15 second⁻¹, at least 20 second⁻¹, or at least 30 second⁻¹ at 25° C.

The post-cleavage (e.g., immolation) reaction may comprise a second order rate constant. For example, the post-cleavage reaction may require a second reducing equivalent from a separate reagent. The post-cleavage reaction may comprise a second order rate constant of at least 0.001 per millimolar (mM⁻¹)hour⁻¹, at least 0.002 mM⁻¹hour⁻¹, at least 0.003 mM⁻¹hour⁻¹, at least 0.004 mM⁻¹hour⁻¹, at least 0.005 mM⁻¹hour⁻¹, at least 0.006 mM⁻¹hour⁻¹, at least 0.008 mM⁻¹hour⁻¹, at least 0.01 mM⁻¹hour⁻¹, at least 0.01 mM⁻¹hour⁻¹, at least 0.02 mM⁻¹hour⁻¹, at least 0.03 mM⁻¹hour⁻¹, at least 0.04 mM⁻¹hour⁻¹, at least 0.05 mM⁻¹hour⁻¹, at least 0.06 mM⁻¹hour⁻¹, at least 0.08 mM⁻¹hour⁻¹, at least 0.1 mM⁻¹hour⁻¹, at least 0.12 mM⁻¹hour⁻¹, at least 0.15 mM⁻¹hour⁻¹, at least 0.2 mM⁻¹hour⁻¹, at least 0.25 mM⁻¹hour⁻¹, at least 0.3 mM⁻¹hour⁻¹, at least 0.4 mM⁻¹hour⁻¹, at least 0.5 mM⁻¹hour⁻¹, at least 0.6 mM⁻¹hour⁻¹, at least 0.7 mM⁻¹hour⁻¹, at least 0.8 mM⁻¹hour⁻¹, at least 0.9 mM⁻¹hour⁻¹, at least 1 mM⁻¹hour⁻¹, at least 2 mM⁻¹hour⁻¹, at least 3 mM⁻¹hour⁻¹, at least 4 mM⁻¹hour⁻¹, at least 5 mM⁻¹hour⁻¹, at least 6 mM⁻¹hour⁻¹, at least 0.12 mM⁻¹minute⁻¹, at least 0.15 mM⁻¹minute⁻¹, at least 0.2 mM⁻¹minute⁻¹, at least 0.25 mM⁻¹minute⁻¹, at least 0.3 mM⁻¹ minute⁻¹, at least 0.4 mM⁻¹minute⁻¹, at least 0.5 mM⁻¹minute⁻¹, at least 0.6 mM⁻¹minute⁻¹, at least 0.7 mM⁻¹minute⁻¹, at least 0.8 mM⁻¹minute⁻¹, at least 0.9 mM⁻¹minute⁻¹, at least 1 mM⁻¹minute⁻¹, at least 2 mM⁻¹minute⁻¹, at least 3 mM⁻¹minute⁻¹, at least 4 mM⁻¹minute⁻¹, at least 5 mM⁻¹minute⁻¹, at least 6 mM⁻¹minute⁻¹, at least 0.12 mM⁻¹ second⁻¹, at least 0.15 mM⁻¹second⁻¹, at least 0.2 mM⁻¹second⁻¹, at least 0.3 mM⁻¹second⁻¹, at least 0.4 mM⁻¹second⁻¹, at least 0.5 mM⁻¹second⁻¹, at least 0.6 mM⁻¹second⁻¹, at least 0.7 mM⁻¹second⁻¹, at least 0.8 mM⁻¹second⁻¹, at least 0.9 mM^{-l}second⁻¹, at least 1 mM⁻¹second⁻¹, at least 1.5 mM⁻¹second⁻¹, at least 2 mM⁻¹second⁻¹, at least 3 mM⁻¹second⁻¹, at least 5 mM⁻¹second⁻¹, at least 8 mM⁻¹second⁻¹, at least 10 mM⁻¹second⁻¹, at least 12 mM⁻¹second⁻¹, at least 15 mM⁻¹second⁻¹, at least 20 mM⁻¹second⁻¹, at least 30 mM⁻¹second⁻¹, 40 mM⁻¹second⁻¹, at least 50 mM⁻¹second⁻¹, at least 60 mM⁻¹second⁻¹, at least 80 mM⁻¹second⁻¹, at least 100 mM⁻¹second⁻¹, at least 120 mM⁻¹second⁻¹, at least 150 mM⁻¹second⁻¹, at least 180 mM⁻¹second⁻¹, at least 200 mM⁻¹second⁻¹, at least 250 mM⁻¹second⁻¹, or at least 300 mM⁻¹ second⁻¹. An immolation reaction may comprise a first order rate constant between 0.01 mM⁻¹min⁻¹ and 0.05 mM⁻¹min⁻¹, between 0.05 mM⁻¹min⁻¹ and 5 mM⁻¹min⁻¹, between 0.1 mM⁻¹min⁻¹ and 10 mM⁻¹min⁻¹, between 1 mM⁻¹min⁻¹ and 20 mM⁻¹min⁻¹, between 10 mM⁻¹min⁻¹ and 50 mM⁻¹min⁻¹, between 1 mM⁻¹min⁻¹ and 100 mM⁻¹min⁻¹, between 10 mM⁻¹min⁻¹ and 200 mM⁻¹min⁻¹, or between 50 mM⁻¹min⁻¹ and 500 mM⁻¹min⁻¹ at 25° C.

A linker may be configured to undergo immolation at less than 0.00001 times the rate of cleavage, at less than 0.00005 times the rate of cleavage, at less than 0.0001 times the rate of cleavage, at less than 0.0005 times the rate of cleavage, at less than 0.001 times the rate of cleavage, at less than 0.005 times the rate of cleavage, at less than 0.01 times the rate of cleavage, at less than 0.05 times the rate of cleavage, at less than 0.1 times the rate of cleavage, at less than 0.5 times the rate of cleavage, at a rate that is about comparable to the rate of cleavage, or at a rate that is faster than cleavage. A rate (e.g., a first or second order rate) may pertain to room temperature, amplification conditions, 1° C. to 10° C., 1° C. to 25° C., 10° C. to 30° C., 1° C. to 45° C., 30° C. to 45° C., 30° C. to 50° C., 50° C. to 80° C., or above 80° C. In some cases, a first or second order rate constant is a room temperature (e.g., 25° C.) rate constant.

A scar resulting from a post-cleavage reaction may comprise favorable properties relative to the scar initially formed post-cleavage. The scar resulting from a post-cleavage reaction may be smaller than the initial scar formed through linker cleavage. The scar resulting from the post-cleavage reaction may be at least 5 Å, at least 10 Å, at least 15 Å, at least 20 Å, or at least 25 Å shorter than the initial scar. The scar resulting from the post-cleavage reaction may comprise no more than 90%, no more than 80%, no more than 70%, no more than 60%, no more than 50%, no more than 40%, or no more than 30% of the length of the initial scar. The scar resulting from the post-cleavage reaction may comprise no more than 90%, no more than 80%, no more than 70%, no more than 60%, no more than 50%, no more than 40%, or no more than 30% of the mass of the initial scar. The scar resulting from the post-cleavage reaction may comprise a molecular weight of less than 300 Daltons, less than 250 Daltons, less than 200 Daltons, 150, less than 100, less than 80, less than 60 Daltons, less than 50 Daltons, less than 40 Daltons, or less than 30 Daltons.

A scar formed from the post-cleavage reaction may further have different properties than the initial scar formed through cleavage. The scar resulting from the post cleavage reaction may comprise a pH that is at least 0.2, at least 0.5, at least 1, at least 1.5, at least 2, at least 2.5, at least 3, at least 3.5, at least 4, at least 4.5, at least 5, at least 5.5, or at least 6 pH units lower than that of the initial scar formed post-cleavage. The scar resulting from the post cleavage reaction may comprise a pH that is at least 0.2, at least 0.5, at least 1, at least 1.5, at least 2, at least 2.5, at least 3, at least 3.5, at least 4, at least 4.5, at least 5, at least 5.5, or at least 6 pH units higher than that of the initial scar formed post-cleavage. The scar resulting from the post cleavage reaction may comprise a dipole moment that is at least 0.2, at least 0.4, at least 0.6, at least 0.8, at least 1, at least 1.2, at least 1.4, at least 1.6, at least 1.8, at least 2.0, at least 2.2, at least 2.4, or at least 2.6 Debye greater than that of the initial scar formed post-cleavage. The scar resulting from the post cleavage reaction may comprise a dipole moment that is at least 0.2, at least 0.4, at least 0.6, at least 0.8, at least 1, at least 1.2, at least 1.4, at least 1.6, at least 1.8, at least 2.0, at least 2.2, at least 2.4, or at least 2.6 Debye lower than that of the initial scar formed post-cleavage. The scar resulting from the post cleavage reaction may comprise a greasy moiety (e.g., a moiety which increases the log P of the scar-containing species), such as a phenyl, an acetylene, a or a naphthalene. The scar resulting from the post cleavage reaction may be configured to couple (e.g., form a bond with or non-covalently adsorb) to a second species. For example, a propargyl amine scar may be configured to chemically couple to a maleimide reagent comprising a greasy substituent.

The activity of an enzyme on a scar-containing species may be dependent on the distribution of positive and negative charges within the scar. For example, an enzyme may comprise a low affinity for negatively charged substrates, or may be inhibited by negatively charged moieties disposed at a specific location on a substrate. Accordingly, a substrate of the present disclosure may be configured to generate a scar with a specific net charge or charge distribution (e.g., a positive charge and a negative charge separated by a defined mean distanced). The scar resulting from the post cleavage reaction may comprise a positive charge. The scar resulting from the post cleavage reaction may comprise a positive net charge. The scar resulting from the post cleavage reaction may comprise a negative charge. The scar resulting from the post cleavage reaction may comprise a net negative charge. The scar resulting from the post cleavage reaction may comprise zwitterionicity. The scar resulting from the post cleavage reaction may be uncharged.

In an aspect, the present disclosure provides a compound of Formula (I):

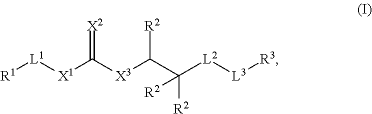

wherein L¹ comprises a linker with a length of at most 50 Angstroms (Å);
R¹ comprises a substrate selected from the group consisting of a nucleotide (e.g., a nucleoside triphosphate or a nucleoside monophosphate), a nucleobase, an amino acid, a protein, a lipid, a cell, and an antibody;
X¹, X², and X³ are independently selected from the group consisting of NR⁴, O, and S;
each instance of R² is independently selected from the group consisting of hydrogen, halogen, ═O, ═S, —CN, —OH, —OR⁵, —N(R⁵)₂, —C(═O)R⁵, —C(═O)OR⁵, —OC(═O)R⁵, —C(═O)N(R⁵)₂, —NR⁵C(═O)R⁵, —NR⁵C(═O)N(R⁵)₂, —OC(═O)N(R⁵)₂, —NR⁵C(═O)OR⁵, —OC(═O)OR⁵, —SRS, —S(═O)R⁵, —SO₂R⁵, —SO₂N(R⁵)₂, —NO₂, optionally substituted C₁-C₆ alkyl, optionally substituted C₂-C₆ alkenyl, optionally substituted C₂-C₆ alkynyl, optionally substituted C₃-C₆ carbocycle, and optionally substituted 3- to 6-membered heterocycle;
or two R² are taken together with the atom or atoms to which they are attached to form an optionally substituted 3- to 8-membered heterocycle or carbocycle;
or two R² on neighboring atoms are absent, such that the neighboring atoms comprise a double bond;
L² comprises a cleavable moiety;
L³ comprises a linker with a length of at least 10 Angstroms (Å);
R³ comprises a detectable moiety;
each instance of R⁴ is independently selected from the group consisting of hydrogen, optionally substituted C₁-C₆ alkyl, optionally substituted C₁-C₆ alkenyl, and optionally substituted C₁-C₆ alkynyl;
each instance of R⁵ is independently selected from the group consisting of halogen, ═O, ═S, —CN, —OH, —OR⁶, —S(═O)R⁶, —SO₂R⁶, —SO₂N(R⁶)₂, —NO₂, optionally substituted C₁-C₆ alkyl, optionally substituted C₂-C₆ alkenyl, optionally substituted C₂-C₆ alkynyl;
or two R⁵ are taken together with the atom or atoms to which they are attached to form a 3- to 6-membered carbocycle or heterocycle; and
each R⁶ is independently selected from hydrogen, halogen, C₁-C₆ alkyl, C₂-C₆ alkenyl, C₂-C₆ alkynyl, and C₁-C₆ haloalkyl.

In some cases, R¹ comprises a substrate selected from the group consisting of a nucleotide (e.g., a nucleoside triphosphate or a nucleoside monophosphate), a nucleobase, an amino acid, and an antibody. In some cases, R¹ comprises a nucleotide. In some cases, L¹ is attached to a nucleobase of R¹ by a bond.

In some cases, L¹ comprises a linker with a length of at least 5 Angstroms (Å). In some cases, L¹ comprises a linker with a length of at least 10 Angstroms (Å). In some cases, L¹ comprises a linker with a length of at least 15 Angstroms (Å). In some cases, L¹ comprises a linker with a length of at least 20 Angstroms (Å). In some cases, L¹ comprises a linker with a length of at least 25 Angstroms (Å). In some cases, L¹ comprises a linker with a length of at least 30 Angstroms (Å). In some cases, L¹ comprises a linker with a length of at least 35 Angstroms (Å). In some cases, L¹ comprises a linker with a length of at least 40 Angstroms (Å). In some cases, L¹ comprises a linker with a length of at most 40 Angstroms (Å). In some cases, L¹ comprises a linker with a length of at most 35 Angstroms (Å). In some cases, L¹ comprises a linker with a length of at most 30 Angstroms (Å). In some cases, L¹ comprises a linker with a length of at most 25 Angstroms (Å). In some cases, L¹ comprises a linker with a length of at most 20 Angstroms (Å). In some cases, L¹ comprises a linker with a length of at most 15 Angstroms (Å). In some cases, L¹ comprises a linker with a length of at most 10 Angstroms (Å). In some cases, L¹ comprises a linker with a length of at most 8 Angstroms (Å). In some cases, L¹ comprises a linker with a length of at most 5 Angstroms (Å). In some cases, L¹ is an optionally substituted C₁-C₈ alkyl, an optionally substituted C₁-C₈ alkenyl, an optionally substituted C₁-C₈ alkynyl, —N(R⁶)—R⁶—, —C(═O)—R⁶—, —C(═O)OR⁶—, —OC(═O)R⁶—, —C(═O)N(R⁶)—R⁶—, —NR⁶C(═O)R⁶—, —NR⁶C(═O)N(R⁶)—R⁶—, —OC(═O)N(R⁶)—R⁶—, —NR⁶C(═O)OR⁶—, —OC(═O)OR⁶—, —SR⁶—, —R⁶—N(R⁶)—R⁶—, —R⁶—O—R⁶—, —R⁶—S—R⁶—, —R⁶—S(═O)—R⁶—, —S(═O)R⁶, —SO₂R⁷, or —SO₂N(R⁶)₂. In some cases, L¹ is an optionally substituted C₁-C₈ alkyl, an optionally substituted C₁-C₈ alkenyl, or an optionally substituted C₁-C₈ alkynyl. In some cases, L¹ is an optionally substituted C₁-C₅ alkyl, an optionally substituted C₁-C₅ alkenyl, or an optionally substituted C₁-C₅ alkynyl. In some cases, L¹ is an optionally substituted C₁-C₃ alkyl, an optionally substituted C₁-C₃ alkenyl, or an optionally substituted C₁-C₃ alkynyl. In some cases, L¹ comprises a propargyl group. In some cases, L¹ is

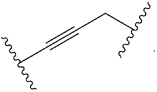 [SMILES: *C#CC*]

In some cases, X₂ is O or S. In some cases, X₂ is O. In some cases, X₂ is O, and X₁ and X₃ are each independently selected from the group consisting of O, NH, and N(CH₃). In some cases, each of X₁, X₂, and X₃ are O. In some cases, X₁ and X₂ are O and X₃ is NH. In some cases, X₂ and X₃ are O, and X₁ is NH.

In some cases, each instance of R² is independently selected from the group consisting of hydrogen, halogen, ═O, ═S, —CN, —OH, —OR⁵, —NH₂, optionally substituted C₁-C₆ alkyl, optionally substituted C₂-C₆ alkenyl, and optionally substituted C₂-C₆ alkynyl, or two R² are taken together with the atom or atoms to which they are attached to form an optionally substituted 3- to 8-membered heterocycle or carbocycle. In some cases, each instance of R² is independently selected from the group consisting of hydrogen, optionally substituted C₁-C₆ alkyl, optionally substituted C₂-C₆ alkenyl, and optionally substituted C₂-C₆ alkynyl. In some cases, each instance of R² is independently selected from the group consisting of hydrogen and C₁-C₄ alkyl. In some cases, only one instance of R² is non-hydrogen. In some cases, two instances of R² are non-hydrogen.

In some cases, L² comprises a reductively cleavable group, an oxidatively cleavable group, an acid-cleavable group, a base-cleavable group, or an enzymatically cleavable group. In some cases, L² comprises a reductively cleavable group, an oxidatively cleavable group, or an enzymatically cleavable group. In some cases, L² comprises a reductively cleavable group. In some cases, L² comprises a disulfide. In some cases, the disulfide is selected from the group consisting of a phenylic disulfide, a benzylic disulfide, a pyridyl disulfide, a naphthyl disulfide, a quinolinyl disulfide, a halo disulfide, a nitro disulfide, and an allylic disulfide. In some cases, the disulfide is selected from the group comprises —S—S—Ar¹—, wherein Ar is selected from the group consisting of optionally substituted aryl and optionally substituted heteroaryl. In some cases, the disulfide comprises

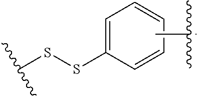

In some cases, L² is stable in the presence of cellular lysate (e.g., bacterial cell lysate or human mesenchymal stromal cell lysate).

In some cases, L³ comprises at least 5 amino acids. In some cases, L³ comprises at least 10 amino acids. In some cases, L³ comprises at least 15 amino acids. In some cases, L³ comprises at least 20 amino acids. In some cases, L³ comprises at least 25 amino acids. In some cases, L³ comprises at least 30 amino acids. In some cases, L³ comprises at least 5 contiguous amino acids. In some cases, L³ comprises at least contiguous 10 amino acids. In some cases, L³ comprises at least 15 contiguous amino acids. In some cases, L³ comprises at least contiguous 20 amino acids. In some cases, L³ comprises at least 25 contiguous amino acids. In some cases, L³ comprises at least 30 contiguous amino acids.

In some cases, the amino acids comprise a non-proteinogenic amino acid. In some cases, L³ comprises at least 5 non-proteinogenic amino acids. In some cases, L³ comprises at least 10 non-proteinogenic amino acids. In some cases, L³ comprises at least 15 non-proteinogenic amino acids. In some cases, L³ comprises at least 20 non-proteinogenic amino acids. In some cases, L³ comprises at least 25 non-proteinogenic amino acids. In some cases, L³ comprises at least 30 non-proteinogenic amino acids. In some cases, L³ comprises at least 5 contiguous non-proteinogenic amino acids. In some cases, L³ comprises at least contiguous 10 non-proteinogenic amino acids. In some cases, L³ comprises at least 15 contiguous non-proteinogenic amino acids. In some cases, L³ comprises at least contiguous 20 non-proteinogenic amino acids. In some cases, L³ comprises at least 25 contiguous non-proteinogenic amino acids. In some cases, L³ comprises at least 30 contiguous non-proteinogenic amino acids. In some cases, the non-proteinogenic amino acids are selected from the group consisting of hydroxyproline and pyroglutamate. In some cases, the non-proteinogenic amino acids are hydroxyprolines.

In some cases, L³ comprises a plurality of amino acids comprising a secondary structural feature. The secondary structural feature may increase the average distance between R¹ and R³ (e.g., between a dye disposed at a first end of the cleavable substrate and a nucleotide disposed at a second end of the cleavable substrate). In some cases, the secondary structural feature comprises a helical structure. In some cases, the helical structure comprises a backbone dihedral angle within 20 degrees of planar. In some cases, the secondary structural feature comprises a helical structure lacking internal hydrogen bonds. In some cases, the secondary structural feature comprises a polyproline helix or a polyproline II helix.

In some cases, L³ comprises a length of at least about 10, at least about 15, at least about 20, at least about 25, at least about 30, at least about 35, at least about 40, at least about 45, at least about 50, at least about 60, at least about 70, at least about 80, at least about 90, or at least about 100 Angstroms (Å). In some cases, L³ comprises a length between about 6 and 12, between about 12 and 24, between about 24 and 36, between about 30 and 50, or between about 40 and 60 Angstroms (Å).

In some cases, L³ comprises a water soluble group. In some cases, the water soluble group is attached to an amino acid of L³. In some cases, the water soluble group is selected from the group consisting of a pyridinium group, an imidazolium group, a quaternary ammonium group, a sulfonate, a phosphate, a hydroxyl, an amine, an imine, a nitrile, an amide, a thiol, a carboxylic acid, a polyether, an aldehyde, a boronic acid, and a boronic ester.

In some cases, R³ comprises an optically detectable moiety, an electrochemically detectable moiety, a chemically detectable moiety, or a mass tag. In some cases, R³ comprises a fluorescent dye, a phosphorescent moiety, a luminescent moiety, an electrochemically detectable moiety, or a mass tag. In some cases, R³ comprises an optically detectable moiety. In some cases, R³ comprises a fluorescent dye or phosphorescent moiety. In some cases, R³ comprises a fluorescent dye. In some cases, R³ comprises a detectable property (e.g., a fluorescence band) that enables identification of R¹.

In some cases, the cleavable substrate is configured to form R¹-L¹-X¹ upon cleavage of L². In some cases, the cleavable substrate is configured to form

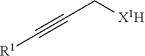

upon cleavage of L². In some cases, the cleavable substrate is configured to form

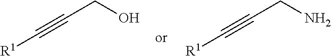

upon cleavage of L¹. In some cases, the cleavable substrate is configured to form

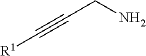

upon cleavage of L¹.

In some cases, a cleavable substrate comprising a structure of Formula (I) comprises the structure of Formula (Ia):

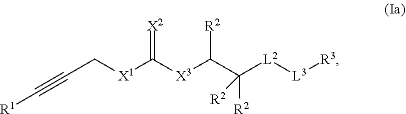

wherein L³ comprises at least 5 amino acids. In some cases, said at least 5 amino acids comprise at least 5 hydroxyprolines.

In some cases, a cleavable substrate comprising the structure of Formula (I) comprises the structure of Formula (Ib):

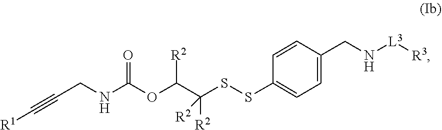

wherein L³ comprises at least 5 amino acids. In some cases, said at least 5 amino acids comprise at least 5 hydroxyprolines.

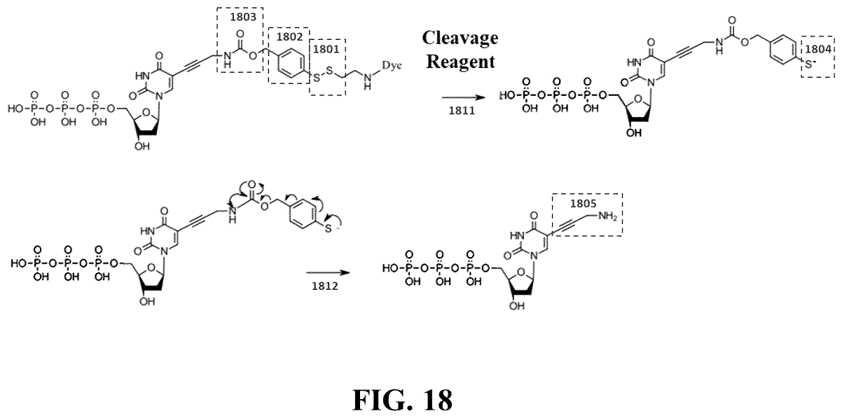

A cleavable linker may comprise a cleavable moiety separated from a first reactive moiety by an olefin, an alkyne, or an aromatic group. For such linkers, cleavage of the cleavable moiety may result in a reaction at the first reactive moiety. An example of such a system is provided in FIG. 18. In this example, a linker comprises a cleavable disulfide 1801 disposed across an aromatic group 1802 from a carbamate first reactive moiety 1803. Following disulfide cleavage 1811 (e.g., by a reducing agent such as THP), the resulting thiol 1804 may facilitate an immolation reaction at the carbamate 1812, resulting in the liberation of a thione and CO₂ and leading to the formation of a propargyl amine product 1805.

### Methods for Using the Labeling Reagents

The use of a labeled substrate comprising a linker (e.g., a detectable moiety-linker-substrate complex such as a dye-linker-nucleoside triphosphate complex) as provided herein may result in more efficient detection during chemical and enzymatic processes (e.g., nucleotide incorporation into a growing nucleic acid strand by a polymerase), compared to a detectable moiety-substrate complex lacking the linker. The increased efficiency may enable a lower amount of the labeled substrate to be used in an assay. Furthermore, the linker may comprise physical properties, such as rigidity, length, and solubility, that enhance a substrate's suitability for an assay.

Cleavable labeled nucleotide substrates (e.g., a nucleoside triphosphate-linker-dye complex) may offer a number of advantages during polymerization. The use of a cleavable, labeled nucleotide substrate provided herein may result in a lower misincorporation rate by a polymerase. The use of a cleavable labeled nucleotide substrate provided herein may result in less loss of template strands, and thus may enable longer sequencing reads. The use of a cleavable labeled nucleotide substrate provided herein may result in diminished mispair extension (e.g., during nucleic acid sequencing). The use of a cleavable labeled nucleotide substrate provided herein may result in a faster polymerization rate relative to the use of non-cleavable labeled nucleotide substrates. In some cases, a chemical or physical property of a nucleotide substrate disposed within a nucleic acid molecule may inhibit subsequent nucleotide incorporations into a growing nucleic acid strand. For example, a labeled nucleotide substrate may comprise a degree of steric bulk that blocks nucleotide binding to a template strand. However, a cleavable labeled nucleotide substrate may be cleaved to remove at least a portion of the linker and, in particular cases, the detectable moiety. Such cleavage may diminish the steric bulk of the nucleotide substrate, and further may provide the nucleotide substrate with chemical and physical properties which more greatly favor further nucleotide incorporations.

In some cases, cleavage of a cleavable labeled nucleotide may leave a scar (e.g., a residual portion of the linker) attached to the nucleotide. Analogously to the aforementioned properties of uncleaved labeled substrates, scar bearing (“scarred”) nucleotides may comprise a physical or chemical property that adversely affects (e.g., inhibits) polymerase activity.

A strategy for mitigating an adverse (e.g., an inhibitory or mispair-inducing) effect of a scar is scar-capping. A physical or chemical property of a scar may be altered by coupling the scar to a capping reagent. The altered property may be favorable (e.g., relative to the uncapped, scarred substrate) for nucleic acid polymerization. For example, the altered physical or chemical property may diminish the inhibitory effect of a scar. The altered physical or chemical property may diminish the rate of nucleotide misincorporation into a growing nucleic acid molecule comprising the capped scar.

Accordingly, a sequencing method may comprise contacting a nucleic acid molecule with a capping reagent. A capping reagent may be selective for a scar, and therefore may be added with a labeled nucleotide substrate, with a cleavage reagent, or subsequent to a cleavage reagent. A nucleic acid polymerization method may comprise a capping reagent addition prior to or following a labeled nucleotide incorporation.

Another strategy for mitigating an adverse effect of a scar is scar immolation. A scar may be configured to undergo a reaction subsequent to cleavage (e.g., an immolation reaction), which may alter a chemical or physical property of the scar. The immolation reaction may be initiated or accelerated by a reagent (e.g., a catalyst or reagent), light, or a condition (e.g., a pH range). The immolation reaction may be spontaneous. The immolation reaction may diminish the size of the scar. For example, an immolation reaction of a thiol-containing scar may result in the loss of the thiol moiety as a thiirane or thietane. As such, an immolation reaction may diminish the steric bulk of a scar. An immolation reaction may alter a chemical or physical property of a scar. For example, a thiol-containing scar may form a more polar and less acidic propargyl amine scar upon immolation.

The methods described herein can be used to reduce dye-dye quenching in multi-dye applications. Hybridization assays can also benefit from linkers that prevent quenching. Quenching effects may result in non-linearity detectable moiety number to signal. The use of a cleavable substrate may result in diminished quenching during polymerization. For example, an assay may comprise nucleotide incorporation steps followed by linker cleavage steps, thereby increasing the likelihood that a growing nucleic acid strand comprises a single dye. The use of a cleavable substrate may diminish background signals during a polymerization reaction by diminishing or limiting the number of fluorescent dyes coupled to a substrate. For example, a method may comprise cleaving the fluorophore from the most recently incorporated nucleotide prior to a subsequent nucleotide addition, thereby limiting the number of fluorophores coupled to a nucleotide during any particular stage of an assay.

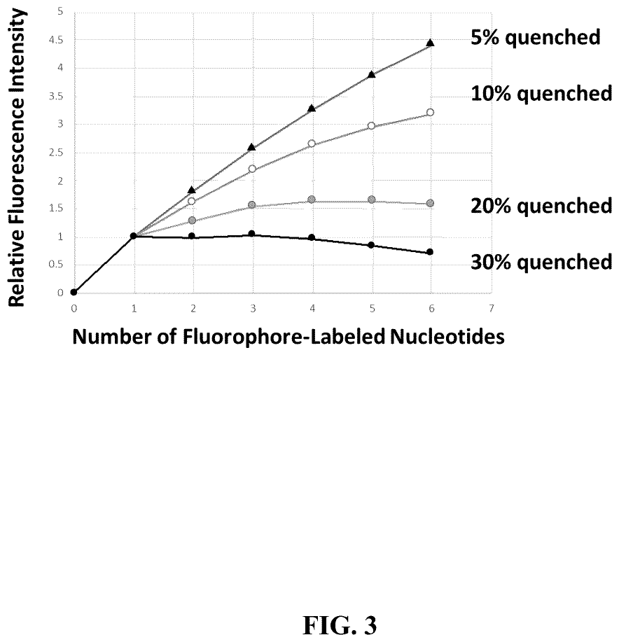

Limiting the number of fluorophores attached to a nucleic acid can greatly enhance measurement accuracy during sequencing or polymerization. In many cases, detecting the presence of a single fluorophore is more accurate than quantifying the number of fluorophores among a plurality disposed within close proximity (e.g., attached to the same nucleic acid molecule or within a single pixel resolved by an imaging technique). A number of factors can make fluorophore quantification challenging. In some cases, the fluorescence signal from a plurality of fluorophores is less than their summed individual intensities. This concept is illustrated in FIG. 3, which shows theoretical fluorescence intensities for various 1- to 6-mer fluorophore-labeled nucleotide homopolymers with 5%, 10%, 20%, and 30% quenching efficiency between fluorophores. As can be seen from the top trace in the plot, even at 5% quenching efficiency, fluorescence intensity increases sub-linearly with homopolymer size (e.g., indicated along the x-axis). For example, at 5% quenching efficiency, a 2-mer containing two fluorophores is predicted to exhibit about 1.8 times the fluorescence of a single fluorophore, a 3-mer only 2.6 times the intensity of a single fluorophore, a 4-mer only 3.3 times the intensity of a single fluorophore, a 5-mer only 3.9 times the intensity of a single fluorophore, and a 6-mer only 4.4 times the intensity of a single fluorophore. At higher quenching efficiencies, such as at 20%, the fluorescence intensity peaks at fewer than 6 fluorophores, illustrating that the addition of a new fluorophore into a molecule can decrease overall fluorescence intensity. Furthermore, multiple fluorophore numbers may provide similar fluorescence intensities that may be difficult to distinguish experimentally. For example, at 20% quenching efficiency, the predicted fluorescence intensities for the 3-, 4-, 5-, and 6-fluorophore molecules are predicted to vary by less than 10%. Thus, limiting the number of fluorophores bound to a molecule (e.g., a growing nucleic acid molecule during polymerization) may increase the accuracy of a fluorophore quantification.

The methods described herein can be used in combination with oligomers and dendrimers for signal amplification. Non-quenching linkers may allow the synthesis of very bright polymers for antibody labeling. These bright antibodies may be used for cell-surface labeling in flow cytometry or for antigen detection methods such as lateral flow tests and fluorescent immunoassays.

The optical (e.g., fluorescent) labeling reagent of the present disclosure may be used as a molecular ruler. The substrate can be a fluorescence quencher, a fluorescence donor, or a fluorescence acceptor. In some cases, the substrate is a nucleotide. The linker can be attached to the nucleotide on the nucleobase as shown below, where the dye is Atto633:

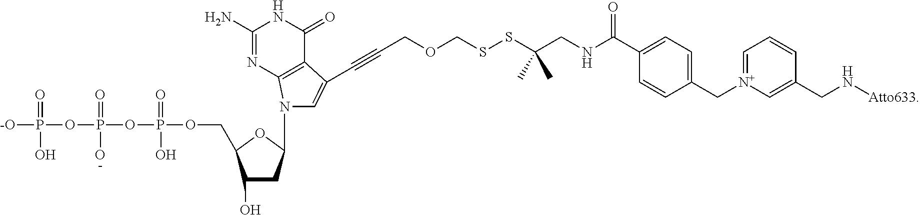

The structure shown above is an optical (e.g., fluorescent) labeling reagent comprising a cleavable (via the disulfide bond) moiety and a fluorescent dye attached via a pyridinium linker to a dGTP analog (dGTP-SS-py-Atto633). Additional examples of optical labeling reagents are provided throughout the disclosure.

The dye-labeled nucleotides described herein can be used in a sequencing by synthesis method using a mixture of dye-labeled and natural nucleotides in a flow-based scheme. Such methods often use a low percentage of labeled nucleotides compared to natural nucleotides. However, using a low percentage of labeled nucleotides compared to natural nucleotides in flow mixtures (e.g., less than 20%) can have multiple drawbacks: (a) since a small fraction of the template provides sequence information, the method requires a high template copy number; (b) variability in DNA polymerase extension rates between labeled and unlabeled nucleotides can result in context-dependent labeling fractions, thus increasing the difficulty of distinguishing a single base incorporation from multiple base incorporations; and (c) the low fraction of labeling moieties can result in high binomial noise in the populations of labeled product. Methods for flow-based sequencing using mostly natural nucleotides are further described in U.S. Pat. No. 8,772,473, which is incorporated herein by reference in its entirety for all purposes.

The semi-rigid linkers provided herein may allow a labeled fraction of dye-labeled nucleotide to natural nucleotide in each flow to be sufficiently high (e.g., 20-100% labeling) to avoid or reduce the effect of the aforementioned disadvantages of such schemes. This higher percentage labeling can result in greater optical (e.g., fluorescent) signal and thus a lower template requirement. If 100% labeling is used, the binomial noise and context variation may be essentially eliminated. The key technical barrier overcome by the solution described herein is that the dye-labeled nucleotides on adjacent or nearby nucleotides must show minimal quenching. The overall result of the combined advantages may be more accurate DNA sequencing.

The present disclosure provides a method for sequencing a nucleic acid molecule. The method can comprise contacting the nucleic acid molecule with a primer under conditions sufficient to hybridize the primer to the nucleic acid molecule, thereby generating a sequencing template. The sequencing template may then be contacted with a polymerase (e.g., as described herein) and a solution (e.g., a nucleotide flow) comprising a plurality of optically (e.g., fluorescently) labeled nucleotides (e.g., as described herein). Each optically labeled nucleotide of the plurality of optically labeled nucleotides may comprise the same chemical structure (e.g., each labeled nucleotide may comprise a dye of a same type, a linker of a same type, and a nucleotide or nucleotide analog of a same type). An optically labeled nucleotide of the plurality of optically labeled nucleotides may be complementary to the nucleic acid molecule at a plurality of positions adjacent to the primer hybridized to the nucleic acid molecule. Accordingly, one or more optically labeled nucleotides of the plurality of optically labeled nucleotides may be incorporated into the sequencing template. Where the nucleic acid molecule includes a homopolymeric region, multiple nucleotides (e.g., a combination of labeled and unlabeled nucleotides) may be incorporated. Incorporation of multiple nucleotides adjacent to one another may be facilitated by the use of non-terminated nucleotides. The solution comprising the plurality of optically labeled nucleotides may then be washed away from the sequencing template (e.g., using a wash flow, as described herein). An optical (e.g., fluorescent) signal from the sequencing template may be measured. Where two or more labeled nucleotides are incorporated into a homopolymeric region, the intensity of the measured optical (e.g., fluorescent) signal may be greater than an optical (e.g., fluorescent) signal that may be measured if a single optically (e.g., fluorescently) labeled nucleotide of the plurality of optically (e.g., fluorescently) labeled nucleotides had been incorporated into the sequencing template. Such a method may be particularly useful for sequencing of homopolymers or portions of nucleic acids that are homopolymeric (i.e., have a plurality of the same base in a row). An optically labeled nucleotide of the plurality of optically labeled nucleotides may comprise a dye (e.g., fluorescent dye) and a linker connected to the dye and a nucleotide (e.g., as described herein). The linker may comprise (i) one or more water soluble groups and (ii) two or more ring systems, wherein at least two of the two or more ring systems are connected to each other by no more than two sp³ carbon atoms, such as by no more than two atoms. The linker may comprise a non-proteinogenic amino acid comprising a ring system of the two or more ring systems. For example, the linker may comprise a hydroxyproline or an amino acid constructed from, e.g., a diamine and a dicarboxylic acid or an amino thiol and a thiol carboxylic acid. The linker may be configured to establish a functional length between the dye and the nucleotide of at least about 0.5 nanometers.

The intensity of the measured optical (e.g., fluorescent) signal may be proportional to the number of optically (e.g., fluorescently) labeled nucleotides incorporated into the sequencing template (e.g., where 100% labeling fraction is used). In other words, quenching may not significantly impact the signal emitted. For example, the intensity may be linearly proportional to the number of optically (e.g., fluorescently) labeled nucleotides incorporated into the sequencing template. The intensity of the measured optical (e.g., fluorescent) signal may be linearly proportional with a slope of approximately 1.0 when plotted against the number of optically (e.g., fluorescently) labeled nucleotides incorporated into the sequencing template. Where less than 100% of substrates are labeled (e.g., less than 100% of nucleotides in a nucleotide flow are labeled), an optical (e.g., fluorescent) signal emitted by substrates (e.g., nucleotides or nucleotide analogs) incorporated into a plurality of growing nucleic acid strands (e.g., a plurality of growing nucleic acid strands coupled to sequencing templates coupled to a support, as described herein) may be proportional to the length of a homopolymer region of the growing nucleic acid strands. Similarly, where less than 100% of substrates are labeled (e.g., less than 100% of nucleotides in each of successive nucleotide flows are labeled), an optical (e.g., fluorescent) signal emitted by substrates (e.g., nucleotides or nucleotide analogs) incorporated into a plurality of growing nucleic acid strands (e.g., a plurality of growing nucleic acid strands coupled to sequencing templates coupled to a support, as described herein) may be proportional to the length of a heteropolymeric and/or homopolymer region of the growing nucleic acid strands. In some such cases, the intensity of a measured optical (e.g., fluorescent) signal may be linearly proportional to the length of a heteropolymeric and/or homopolymeric region into which substrates have incorporated. For example, a measured optical (e.g., fluorescent) signal may be linearly proportional with a slope of approximately 1.0 when optical (e.g., fluorescent) signal is plotted against the length in substrates of a heteropolymeric and/or homopolymeric region into which substrates have incorporated

The solution comprising the plurality of optically (e.g., fluorescently) labeled nucleotides may also contain un-labeled nucleotides (e.g., the labeling fraction may be less than 100%). For example, at least about 20% of nucleotides in the solution may be optically labeled, and at least about 80% of nucleotides in the solution may not be optically labeled. In some cases, the majority of the nucleotides in the solution may be optically labeled (e.g., between about 50-100%).

In some cases, two or more optically (e.g., fluorescently) labeled nucleotides of the plurality of optically (e.g., fluorescently) labeled nucleotides are incorporated into the sequencing template (e.g., into a homopolymeric region). In some cases, three or more optically (e.g., fluorescently) labeled nucleotides of the plurality of optically (e.g., fluorescently) labeled nucleotides are incorporated into the sequencing template. The number of optically labeled nucleotides incorporated into the sequencing template during a given nucleotide flow may depend on the homopolymeric nature of the nucleic acid molecule. In some cases, a first optically (e.g., fluorescently) labeled nucleotide of the plurality of optically (e.g., fluorescently) labeled nucleotides is incorporated within four positions of a second optically (e.g., fluorescently) labeled nucleotide of the plurality of optically (e.g., fluorescently) labeled nucleotides.

An optically (e.g., fluorescently) labeled nucleotide may comprise a cleavable group to facilitate cleavage of the optical (e.g., fluorescent) label (e.g., as described herein). In some cases, a method may further comprise, subsequent to incorporation of the one or more optically (e.g., fluorescently) labeled nucleotides and washing away of residual solution, cleaving optical (e.g., fluorescent) labels of the one or more optically (e.g., fluorescently) labeled nucleotides incorporated into the sequencing template (e.g., as described herein). The cleavage flow may be followed by an additional wash flow.

In some cases, a nucleotide flow and wash flow may be followed by a “chase” flow comprising unlabeled nucleotides and no labeled nucleotides. The chase flow may be used to complete the sequencing reaction for a given nucleotide position or positions of the sequencing template (e.g., across a plurality of such templates immobilized to a support). The chase flow may precede detection of an optical signal from a template. Alternatively, the chase flow may follow detection of an optical signal from a template. The chase flow may precede a cleavage flow. Alternatively, the chase flow may follow a cleavage flow. The chase flow may be followed by a wash flow.

The methods provided herein can also be used to sequence heteropolymers and/or heteropolymeric regions of a nucleic acid molecule (i.e., portions that are not homopolymeric). Accordingly, the methods described herein can be used to sequence a nucleic acid molecule having any degree of heteropolymeric or homopolymeric nature.

Regarding homopolymers, a nucleotide flow at a homopolymer region may incorporate several nucleotides in a row. Contacting a sequencing template comprising a nucleic acid molecule (e.g., a nucleic acid molecule hybridized to an unextended primer) comprising a homopolymer region with a solution comprising a plurality of nucleotides (e.g., a combination of labeled and unlabeled nucleotides), where each nucleotide of the plurality of nucleotides is of a same type, may result in multiple nucleotides of the plurality of nucleotides being incorporated into the sequencing template. In some cases, at least 2, at least 3, at least 4, at least 5, at least 6, at least 7, at least 8, at least 9, or at least 10 nucleotides are incorporated (i.e., in a homopolymeric region of a nucleic acid molecule). The plurality of nucleotides incorporated into the sequencing template may comprise a plurality of labeled nucleotides (e.g., optically labeled, such as fluorescently labeled), as described herein. In such an instance, one or more of said nucleotides incorporated into a homopolymer region may be labeled and may either occupy adjacent or non-adjacent positions to other labeled nucleotides incorporated into the homopolymeric region. The intensity of a signal obtained from a nucleic acid molecule may be proportional to the number of incorporated labeled nucleotides (e.g., where a labeling fraction of 100% is used). For example, the intensity of an optical signal (e.g., fluorescent signal) obtained from a nucleic acid molecule containing two labeled nucleotides may be of greater intensity than the optical signal obtained from a nucleic acid molecule containing one labeled nucleotide. Furthermore, the intensity of a signal obtained from a nucleic acid molecule may depend on the relative positioning of labeled nucleotides within a nucleic acid molecule. For example, a nucleic acid molecule containing two labeled nucleotides in non-adjacent positions may provide a different signal intensity than a nucleic acid molecule containing two labeled nucleotides in adjacent positions. Quenching in such systems may be optimized by careful selection of linkers and dyes (e.g., fluorescent dyes). In some cases, a plot of optical signal (e.g., fluorescence) vs. homopolymer length can be linear. For example, measured optical signal for an ensemble of growing nucleic acid strands including homopolymeric regions into which labeled nucleotides are incorporated may be approximately linearly proportional to the nucleotide length of the homopolymeric region.

In another aspect, the present disclosure provides a method for sequencing a nucleic acid molecule. The method can comprise contacting the nucleic acid molecule with a primer under conditions sufficient to hybridize the primer to the nucleic acid molecule, thereby generating a sequencing template. The nucleic acid molecule may then be contacted with a polymerase and a first solution comprising a plurality of first optically (e.g., fluorescently) labeled nucleotides (and, optionally, a plurality of first unlabeled nucleotides). Each first optically (e.g., fluorescently) labeled nucleotide of the plurality of first optically (e.g., fluorescently) labeled nucleotides is of a same type. A first optically (e.g., fluorescently) labeled nucleotide of the plurality of first optically (e.g., fluorescently) labeled nucleotides may be complementary to the nucleic acid molecule to be sequenced at a position adjacent to the primer. A first optically (e.g., fluorescently) labeled nucleotide of the plurality of first optically (e.g., fluorescently) labeled nucleotides may thus be incorporated into the sequencing template to generate an extended primer. The first solution comprising the plurality of first optically (e.g., fluorescently) labeled nucleotides may then be washed away from the sequencing template (e.g., using a wash solution). A first optical (e.g., fluorescent) signal emitted by the sequencing template may then be measured (e.g., as described herein). The sequencing template may then be contacted with a polymerase and a second solution comprising a plurality of second optically (e.g., fluorescently) labeled nucleotides (and, optionally, a plurality of second unlabeled nucleotides). Each second optically (e.g., fluorescently) labeled nucleotide of the plurality of second optically (e.g., fluorescently) labeled nucleotides may be of a same type. A second optically (e.g., fluorescently) labeled nucleotide of the plurality of second optically (e.g., fluorescently) labeled nucleotides may be complementary to the nucleic acid molecule to be sequenced at a position adjacent to the extended primer. A second optically (e.g., fluorescently) labeled nucleotide of the plurality of second optically (e.g., fluorescently) labeled nucleotides may thus be incorporated into the sequencing template. The second solution comprising the plurality of second optically (e.g., fluorescently) labeled nucleotides may then be washed away from the sequencing template. A second optical (e.g., fluorescent) signal emitted by the sequencing template may then be measured. In some cases, the intensity of the second optical (e.g., fluorescent) signal may be greater than the intensity of the first optical (e.g., fluorescent) signal.

A first optically labeled nucleotide of the plurality of first optically labeled nucleotides may comprise a first dye (e.g., fluorescent dye) and a first linker connected to the first dye and a first nucleotide (e.g., as described herein). Similarly, a second optically labeled nucleotide of the plurality of second optically labeled nucleotides may comprise a second dye (e.g., fluorescent dye) and a second linker connected to the second dye and a second nucleotide (e.g., as described herein). The first linker may comprise (i) one or more water soluble groups and (ii) two or more ring systems, wherein at least two of the two or more ring systems are connected to each other by no more than two sp³ carbon atoms, such as by no more than two atoms. For example, at least two of the two or more ring systems may be connected to each other by an sp² carbon atom. The linker may comprise a non-proteinogenic amino acid comprising a ring system of the two or more ring systems. For example, the first linker may comprise one or more hydroxyproline moieties (e.g., as described herein). The first linker may be configured to establish a functional length between the first dye and the first nucleotide of at least about 0.5 nanometers. Similarly, the second linker may comprise (i) one or more water soluble groups and (ii) two or more ring systems, wherein at least two of the two or more ring systems are connected to each other by no more than two sp³ carbon atoms, such as by no more than two atoms. For example, at least two of the two or more ring systems may be connected to each other by an sp² carbon atom. The linker may comprise a non-proteinogenic amino acid comprising a ring system of the two or more ring systems. For example, the second linker may comprise one or more hydroxyproline moieties (e.g., as described herein). The second linker may be configured to establish a functional length between the second dye and the second nucleotide of at least about 0.5 nanometers. The first linker and the second linker may have the same structure. Alternatively, the first linker and the second linker may have different structures. The first linker and the second linker may comprise a shared structural motif, such as a shared cleavable component (e.g., as described herein). The first linker and/or the second linker may comprise a cleavable group configured to be cleaved with a cleavage reagent (e.g., as described herein).

Cleavage of the cleavable group of the first linker and/or second linker may generate a scar. The scar may comprise a thiol. The scar may undergo an immolation reaction to yield an immolated scar. The immolated scar may comprise a primary amine or a primary hydroxyl moiety. The primary amine moiety may comprise a propargyl amine. The primary hydroxyl moiety may comprise a propargyl alcohol. The scar may be capped with a capping reagent. The capping reagent may comprise a disulfide configured to react with the scar thiol.

The first solution comprising the plurality of first optically (e.g., fluorescently) labeled nucleotides may also contain first un-labeled nucleotides. For example, about 20% of the nucleotides of the first solution may be un-labeled. In some cases, at least 20% of the nucleotides of the first solution may be optically labeled, such as at least 50% or at least 80%. The un-labeled nucleotides may comprise the same nucleotide moiety (e.g., canonical nucleotide moiety) as the optically labeled nucleotides. Similarly, the second solution comprising the plurality of first optically labeled nucleotides may also contain second un-labeled nucleotides. For example, about 20% of the nucleotides of the second solution may be un-labeled. In some cases, at least 20% of the nucleotides of the second solution may be optically labeled, such as at least 50% or at least 80%. The un-labeled nucleotides may comprise the same nucleotide moiety (e.g., canonical nucleotide moiety) as the optically labeled nucleotides.

The plurality of first optically (e.g., fluorescently) labeled nucleotides may be different than the plurality of second optically (e.g., fluorescently) labeled nucleotides. For example, the plurality of first optically (e.g., fluorescently) labeled and the plurality of second optically (e.g., fluorescently) labeled nucleotides may comprise the same optical (e.g., fluorescent) label (e.g., the same dye) and different nucleotides. Alternatively, the plurality of first optically (e.g., fluorescently) labeled and the plurality of second optically (e.g., fluorescently) labeled nucleotides may comprise different optical (e.g., fluorescent) labels (e.g., different dyes) and the same nucleotides. In some cases, the plurality of first optically (e.g., fluorescently) labeled and the plurality of second optically (e.g., fluorescently) labeled nucleotides may comprise different optical (e.g., fluorescent) labels (e.g., different dyes) and different nucleotides. The first dye of the first plurality of optically labeled nucleotides and the second dye of the second plurality of optically labeled nucleotides may emit signal at approximately the same wavelength or range of wavelengths (e.g., whether the first and second dyes have the same or different chemical structures). For example, the first dye and the second dye may both emit signal in the green region of the visible portion of the electromagnetic spectrum.

In some cases, two or more first optically (e.g., fluorescently) labeled nucleotides may be incorporated into the sequencing template (e.g., in a homopolymeric region of the nucleic acid molecule). In some cases, two or more second optically (e.g., fluorescently) labeled nucleotides may be incorporated into the sequencing template.

Additional optically (e.g., fluorescently) labeled nucleotides may also be provided and incorporated into the sequencing template (e.g., in successive nucleotide flows, as described herein). For example, the method may further comprise contacting the sequencing template with a polymerase and a third solution comprising a plurality of third optically (e.g., fluorescently) labeled nucleotides, wherein each third optically (e.g., fluorescently) labeled nucleotide of the plurality of third optically (e.g., fluorescently) labeled nucleotides is of a same type, and wherein a third optically (e.g., fluorescently) labeled nucleotide of the plurality of third optically (e.g., fluorescently) labeled nucleotides is complementary to the nucleic acid molecule at a position adjacent to the further extended primer hybridized to the nucleic acid molecule, thereby incorporating a third optically (e.g., fluorescently) labeled nucleotide of the plurality of third optically (e.g., fluorescently) labeled nucleotides into the sequencing template; washing the third solution comprising the plurality of third optically (e.g., fluorescently) labeled nucleotides away from the sequencing template; and measuring a third optical (e.g., fluorescent) signal emitted by the sequencing template. In some cases, the intensity of the third optical signal may be greater than the intensity of the first optical (e.g., fluorescent) signal and the intensity of the second optical (e.g., fluorescent) signal. This process may be repeated with a fourth solution, etc. The third and fourth solutions may comprise optically (e.g., fluorescently) labeled nucleotides having different nucleotides than the first and second solutions, such that each canonical nucleotide (A, C, G, and U/T) may be provided in sequence to the sequencing template. A cycle in which each canonical nucleotide is provided to the sequencing template may be repeated one or more times to sequence and/or amplify the nucleic acid molecule.

A third optically labeled nucleotide of the plurality of third optically labeled nucleotides may comprise a third dye (e.g., fluorescent dye) and a third linker connected to the third dye and a third nucleotide (e.g., as described herein). The third linker may comprise a cleavable group, cleavage of which may generate a scar coupled to the nucleotide. The third linker may comprise (i) one or more water soluble groups and (ii) two or more ring systems, wherein at least two of the two or more ring systems are connected to each other by no more than two sp³ carbon atoms, such as by no more than two atoms. For example, at least two of the two or more ring systems may be connected to each other by an sp² carbon atom. The linker may comprise a non-proteinogenic amino acid comprising a ring system of the two or more ring systems. For example, the third linker may comprise one or more hydroxyproline moieties (e.g., as described herein). The third linker may be configured to establish a functional length between the third dye and the third nucleotide of at least about 0.5 nanometers. The third linker and the first linker may have the same or different structures. Similarly, the third linker and the second linker may have the same or different structures. The third dye may have the same or a different structure as the first dye. Similarly, the third dye may have the same or a different structure as the second dye. The third dye and the first and/or second dye may emit at approximately the same wavelength or range of wavelengths (e.g., whether these dyes have the same or different chemical structures). Further, the third nucleotide may be of a same or different type as the first nucleotide, or the third nucleotide may be of a same or different type as the second nucleotide.

The method may further comprise, subsequent to washing a given solution (e.g., nucleotide flow) away (e.g., using a wash solution), cleaving the optical (e.g., fluorescent) label of its respective nucleotides. For example, after the first solution is washed away, the optical (e.g., fluorescent) label of the first optically (e.g., fluorescently) labeled nucleotide incorporated into the sequencing template may be cleaved (e.g., using a cleavage reagent to cleave a cleavable group of a linker of the first optically labeled nucleotide, as described herein). For example, the fluorescent dye(s) of the first optically labeled nucleotide(s) incorporated into the sequencing template may be cleaved prior to contacting the sequencing template with second optically labeled nucleotides (e.g., in a second nucleotide flow, as described herein). Accordingly, signal may be detected from one or more first optically labeled nucleotides prior to incorporation of one or more second optically labeled nucleotides into the sequencing template. Separation of the fluorescent dye(s) of the first optically labeled nucleotide(s) incorporated into the sequencing template may provide a scar comprising a portion of the linker coupled to the first optically labeled nucleotide, or a derivative thereof. The scar may undergo an immolation reaction, yielding an immolating scar which may comprise different physical (e.g., size) and chemical (e.g., pKa) properties. Similarly, after the second solution (e.g., second nucleotide flow) is washed away, the optical (e.g., fluorescent) label of the second optically (e.g., fluorescently) labeled nucleotide incorporated into the sequencing template may be cleaved. Similarly, a scar generated by the cleavage of the second optically labeled nucleotide may undergo an immolation reaction. The immolation reaction may be spontaneous or may be initiated by a reagent, light, energy (e.g., an electrical potential) or a change in condition (e.g., a change in pH or temperature). All or a portion of the first and second linkers may be cleaved during the respective cleaving processes.

The method may further comprise contacting a nucleotide with a capping reagent. The capping reagent may couple to a scar, for example by covalently binding to the scar. The capping reagent may be added with a labeled nucleotide, with a cleavage reagent, subsequent to a cleavage reagent, or subsequent to a reagent, light-input, energy-input, or change in condition for a scar immolation reaction. The capping reagent may be added subsequent to a labeled nucleotide. The capping reagent may be added with an unlabeled nucleotide. For example, a method may comprise first contacting a nucleic acid with a labeled nucleotide, and then subsequently contacting the nucleic acid with a capping reagent and an unlabeled nucleotide of the same canonical type as the labeled nucleotide. Such a method may increase the likelihood of complete extension across homopolymeric regions of a template nucleic acid. The capping reagent may remain stably bound to the scarred nucleotide through subsequent nucleotide additions and cleavage steps.

In another aspect, provided herein is a method for sequencing a nucleic acid molecule. The method can comprise providing a solution comprising a plurality of optically (e.g., fluorescently) labeled nucleotides, wherein each optically (e.g., fluorescently) labeled nucleotide of the plurality of optically (e.g., fluorescently) labeled nucleotides is of a same type. A given optically (e.g., fluorescently) labeled nucleotide of the plurality of fluorescently labeled nucleotides may comprise an optical (e.g., fluorescent) dye that is connected to a nucleotide via a semi-rigid water-soluble linker having a defined molecular weight. The linker connecting the dye and nucleotide may provide a functional length of at least about 0.5 nanometers (nm) between the dye and nucleotide. The nucleic acid molecule may then be contacted with a primer under conditions sufficient to hybridize the primer to a nucleic acid molecule to be sequenced to generate a sequencing template. The sequencing template may then be contacted with a polymerase and the solution containing the plurality of optically (e.g., fluorescently) labeled nucleotides, wherein an optically (e.g., fluorescently) labeled nucleotide of the plurality of optically (e.g., fluorescently) labeled nucleotides is complementary to the nucleic acid molecule to be sequenced at a position adjacent to the primer. One or more optically (e.g., fluorescently) labeled nucleotides of the plurality of optically (e.g., fluorescently) labeled nucleotides may thus be incorporated into the sequencing template. The solution comprising the plurality of optically (e.g., fluorescently) labeled nucleotides may be washed away from the sequencing template (e.g., using a wash solution). An optical (e.g., fluorescent) signal emitted by the sequencing template may then be measured.

The linker may comprise (i) one or more water soluble groups and (ii) two or more ring systems, wherein at least two of the two or more ring systems are connected to each other by no more than two sp³ carbon atoms, such as by no more than two atoms (e.g., as described herein). For example, at least two of the two or more ring systems may be connected to each other by an sp² carbon atom. The linker may comprise a non-proteinogenic amino acid comprising a ring system of the two or more ring systems. For example, the linker may comprise one or more hydroxyproline moieties (e.g., as described herein). The linker may establish a functional length between the fluorescent dye and the nucleotide of at least about 0.5 nanometers (e.g., as described herein).

The measured optical (e.g., fluorescent) signal may be proportional to the number of optically (e.g., fluorescently) labeled nucleotides that were incorporated into the sequencing template. For example, where 100% labeling fraction is used (e.g., all nucleotides in the solution are labeled), quenching may not diminish the emitted signal. In such a system, the measured optical (e.g., fluorescent) signal can be linearly proportional to the number of optically (e.g., fluorescently) labeled nucleotides that were incorporated into the sequencing template. The measured optical (e.g., fluorescent) signal may be linearly proportional with a slope of approximately 1.0 when plotted against the number of optically (e.g., fluorescently) labeled nucleotides that were incorporated into the sequencing template. Where less than 100% of nucleotides are labeled (e.g., less than 100% of nucleotides in the solution are labeled), an optical (e.g., fluorescent) signal emitted by nucleotides incorporated into a plurality of growing nucleic acid strands (e.g., a plurality of growing nucleic acid strands coupled to sequencing templates coupled to a support, as described herein) may be proportional to the length of a homopolymer region of the growing nucleic acid strands. Similarly, where less than 100% of nucleotides are labeled, an optical (e.g., fluorescent) signal emitted by nucleotides incorporated into a plurality of growing nucleic acid strands (e.g., a plurality of growing nucleic acid strands coupled to sequencing templates coupled to a support, as described herein) may be proportional to the length of a heteropolymeric and/or homopolymer region of the growing nucleic acid strands. In some such cases, the intensity of a measured optical (e.g., fluorescent) signal may be linearly proportional to the length of a heteropolymeric and/or homopolymeric region into which nucleotides have incorporated. For example, a measured optical (e.g., fluorescent) signal may be linearly proportional with a slope of approximately 1.0 when optical (e.g., fluorescent) signal is plotted against the length in nucleotides of a heteropolymeric and/or homopolymeric region into which nucleotides have incorporated

In some cases, the solution containing an optically (e.g., fluorescently) labeled nucleotide also contains un-labeled nucleotides. The un-labeled nucleotides may comprise the same nucleotide moiety (e.g., the same canonical nucleotide). In some embodiments, about 5%, about 10%, about 20%, about 30%, about 40%, about 50%, about 60%, about 70%, about 80%, about 90%, or about 100% of nucleotides in the solution are fluorescently labeled. In some cases, at least about 5%, at least about 10%, at least about 20%, at least about 30%, at least about 40%, at least about 50%, at least about 60%, at least about 70%, at least about 80%, at least about 90%, or more of nucleotides in the solution are fluorescently labeled. In some cases, at least about 5%, at least about 10%, at least about 20%, at least about 30%, at least about 40%, at least about 50%, at least about 60%, at least about 70%, at least about 80%, at least about 90%, or more of nucleotides in the solution are not fluorescently labeled.

A plurality of labeled nucleotides can be incorporated at locations along a nucleic acid molecule in proximity to each other. In some cases, a first optically (e.g., fluorescently) labeled nucleotide is incorporated within 4 positions, within 3 positions, within 2 positions, or next to a second optically (e.g., fluorescently) labeled nucleotide (e.g., a second optically labeled nucleotide of a same or different nucleotide type). In some cases, the method further comprises cleaving the optical (e.g., fluorescent) labels from the nucleotides after measuring the optical (e.g., fluorescent) signal (e.g., as described herein). Cleaving an optical (e.g., fluorescent) label may leave behind a scar (e.g., as described herein). A nucleic acid sequencing assay may be used to evaluate dye-labeled nucleotides. The assay may use a nucleic acid template having a known sequence, which sequence may include one or more homopolymeric regions. The template may be immobilized to a support (e.g., as described herein) via an adapter. A primer having a sequence at least partly complementary to the adapter or a portion thereof may hybridize to the adapter or portion thereof and provide a starting point for generation of a nucleic acid strand having a sequence complementary to that of the template via incorporation of labeled and unlabeled nucleotides (e.g., as described herein). The sequencing assay may use four distinct four nucleotide flows including different canonical nucleobases that may be repeated in cyclical fashion (e.g., cycle 1: A, G, C, U; cycle 2 A, G, C, U; etc.). Each nucleotide flow may include nucleotides including nucleobases of a single canonical type (or analogs thereof), some of which may be include optical labeling reagents provided herein. The labeling fraction (e.g., % of nucleotides included in the flow that are attached to an optical labeling reagent) may be varied between, e.g., 0.5% to 100%. Labeling fractions may be different for different nucleotide flows. Nucleotides may not be terminated to facilitate incorporation into homopolymeric regions. The template may be contacted with a nucleotide flow, followed by one or more wash flows (e.g., as described herein). The template may also be contacted with a cleavage flow (e.g., as described herein) including a cleavage reagent configured to cleave a portion of the optical labeling reagents attached to labeled nucleotides incorporated into the growing nucleic acid strand. A wash flow may be used to remove cleavage reagent and prepare the template for contact with a subsequent nucleotide flow. Emission may be detected from labeled nucleotides incorporated into the growing nucleic acid strand after each nucleotide flow.

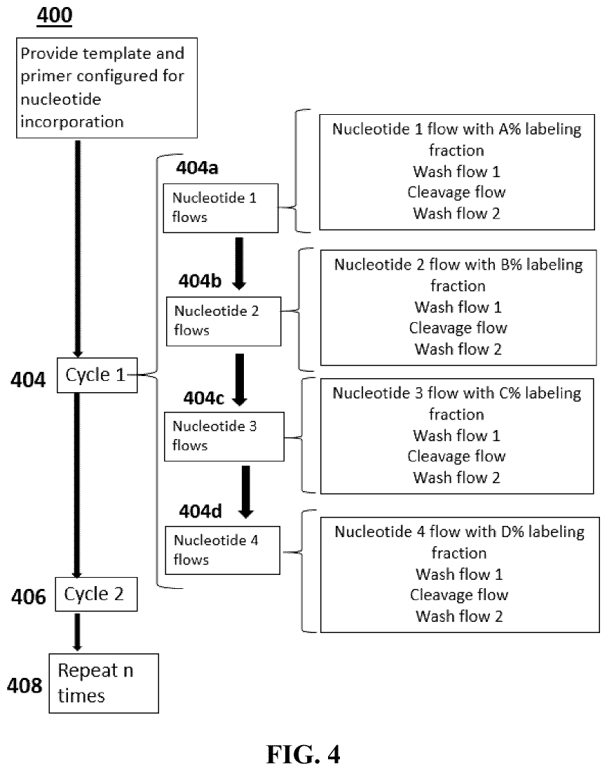

An example sequencing procedure 400 is provided in FIG. 4, in which a template and primer configured for nucleotide incorporation are provided for sequencing. A first sequencing cycle 404 is subsequently performed. First sequencing cycle 404 includes four flow processes 404a, 404b, 404c, and 404d, each of which consists of multiple flows. Nucleotides 1, 2, 3, and 4 may each include nucleobases of different canonical types (e.g., A, G, C, and U). A given nucleotide flow may include both labeled nucleotides (e.g., nucleotides labeled with an optical labeling reagent provided herein) and unlabeled nucleotides. The labeling fraction of each nucleotide flow may be different. That is, the percentages A, B, C, and D in FIG. 4 may be the same or different and may each range from 0% to 100% (e.g., as described herein). Labels and linkers used to label nucleotides 1, 2, 3, and 4 may be of the same or different types. For example, nucleotide 1 may have a linker including a cleavable linker and a hyp10 linker and a first green dye, and nucleotide 2 may have a linker including a cleavable linker but not a hyp10 linker and a second green dye. The first green dye may be the same as or different than the first green dye. The cleavable linkers associated with the different nucleotides may be the same or different. Flow process 404a may include a nucleotide flow (e.g., a flow including a plurality of nucleotides of type Nucleotide 1, A % of which may be labeled). During this flow, labeled and unlabeled nucleotides may be incorporated into the growing strand (e.g., using a polymerase enzyme). A first wash flow (“wash flow 1”) may be used to remove unincorporated nucleotides and associated reagents. A cleavage flow including a cleavage reagent may be provided to all or portions of the optical labeling reagents attached to incorporated nucleotides. For example, labeled nucleotides may include a cleavable linker portion that may by cleaved upon contact with the cleavage reagent to provide a scarred nucleotide.

The scarred nucleotide may undergo an immolation reaction that yields an immolated scar or entirely excises the scar from the nucleotide. A scar of the scarred nucleotide may be capped by a capping reagent. The capping reagent may be added in the cleavage flow, in between the cleavage flow and wash flow 2 (e.g., in a distinct capping reagent flow), in wash flow 2, or subsequent to wash flow 2. The immolation or capping of the nucleotide may increase a rate of a subsequent nucleotide incorporation. For example, incorporation of a first nucleotide adjacent to (e.g., directly next to, or within 1, 2, 3, or 4 nucleotides of) a scarred, non-immolated, non-capped nucleotide may occur at less than 90% the rate, less than 80% the rate, less than 70% the rate, less than 60% the rate, less than 50% the rate, less than 40% the rate, less than 30% the rate, less than 25% the rate, less than 20% the rate, less than 15% the rate, less than 10% the rate, less than 8% the rate, less than 6% the rate, less than 5% the rate, less than 4% the rate, less than 3% the rate, less than 2% the rate, less than 1% the rate, less than 0.75% the rate, less than 0.5% the rate, less than 0.25% the rate, less than 0.1% the rate, less than 0.05% the rate, or less than 0.010% the rate of an incorporation next to a capped or immolated nucleotide of the same canonical type. Incorporation of a first nucleotide adjacent to an immolated or capped scar may occur at least at 90% the rate, at least 80% the rate, at least 70% the rate, at least 60% the rate, at least 50% the rate, at least 40% the rate, at least 30% the rate, at least 25% the rate, at least 20% the rate, at least 15% the rate, at least 10% the rate, at least 8% the rate, at least 6% the rate, at least 5% the rate, at least 4% the rate, at least 3% the rate, at least 2% the rate, at least 1% the rate, at least 0.75% the rate, at least 0.5% the rate, at least 0.25% the rate, at least 0.1% the rate, at least 0.05% the rate, or at least 0.01% the rate of an incorporation next to an unlabeled, unscarred nucleotide of the same canonical type.

A second wash flow (“wash flow 2”) may be used to remove the cleavage reagent and cleaved materials. Nucleotide flow process 404a may also include a “chase” process in which a nucleotide flow including only unlabeled nucleotides of type Nucleotide 1 may be flowed. Such a chase process may be followed by a wash flow. The chase process and its accompanying wash flow may take place after the initial nucleotide flow and wash flow 1, or after the cleavage flow and wash flow 2. The next nucleotide flow process 404b may then begin and proceed in similar fashion. Following completion of processes 404b, 404c, and 404d, the first flow cycle 404 may be complete. A second flow cycle 406 may begin. Cycle 406 may include the same flow processes as described above with regards to the first flow cycle 404 in the same or different order. Additional flow cycles may be performed 408 until all or a portion of the template has been sequenced. Detection of incorporated nucleotides via emission detection may be performed after nucleotide flows and initial wash flows and before cleavage flows for each nucleotide flow process (e.g., flow process 404a may include a detection process between wash flow 1 and cleavage flow, etc.). A template interrogated by such a sequencing process may be immobilized to a support (e.g., as described herein). A plurality of such templates (e.g., at least about 100, 200, 500, 1000, 10000, 100,000, 500,000, 1,000,000, or more templates) may be interrogated contemporaneously in this fashion (e.g., in clonal fashion). In such a system, incorporation of nucleotides may be detected as an average over the plurality of templates, which may permit the use of labeling fractions of less than 100%.

In some cases, for any of the preceding methods, the nucleotide is guanosine (G) and the linker decreases quenching between the nucleotide and the dye (e.g., fluorescent) dye.

In some cases, for any of the preceding methods, an optically (e.g., fluorescently) labeled nucleotide comprising a linker provided herein is more efficiently incorporated into a sequencing template than another optically (e.g., fluorescently) labeled nucleotide that comprises the same nucleotide and optical (e.g., fluorescent) dye but does not include the linker. In some cases, for any of the preceding methods, an optically (e.g., fluorescently) labeled nucleotide comprising a linker provided herein is incorporated into a sequencing template with higher fidelity than another optically (e.g., fluorescently) labeled nucleotide that comprises the same nucleotide and optical (e.g., fluorescent) dye but does not include the linker.

For any of the sequencing methods provided herein, the polymerase used may be a Family A polymerase such as Taq, Klenow, or Bst polymerase. Alternatively, for any of the sequencing methods provided herein, the polymerase may be a Family B polymerase such as Vent(exo-) or Therminator™ polymerase.

In an aspect, the present disclosure provides methods for sequencing a nucleic acid molecule using the optically (e.g., fluorescently) labeled nucleotides described herein. A method may comprise providing a plurality of nucleic acid molecules, which plurality of nucleic acid molecules may comprise or be part of a colony or a plurality of colonies. The plurality of nucleic acid molecules may have sequence homology to a template sequence. The method may comprise contacting the plurality of nucleic acid molecules with a solution comprising a plurality of nucleotides (e.g., a solution comprising a plurality of optically labeled nucleotides) under conditions sufficient to incorporate a subset of the plurality of nucleotides into a plurality of growing nucleic acid strands that is complementary to the plurality of nucleic acid molecules. In some instances, at least about 20% of the subset of the plurality of nucleotides are optically (e.g., fluorescently) labeled nucleotides (e.g., as described herein). The method may comprise detecting one or more signals or signal changes from the labeled nucleotides incorporated into the plurality of growing nucleic acid strands, wherein the one or more signals or signal changes are indicative of the labeled nucleotides having incorporated into the plurality of growing nucleic acid strands.

The optically (e.g., fluorescently) labeled nucleotides of the plurality of nucleotides may be non-terminated. In such cases, the growing strands may incorporate one or more consecutive nucleotides during (e.g., a complimentary base to the plurality of nucleotides in solution is not present at a plurality of positions adjacent to the primer hybridized to the nucleic acid molecule). The one or more signals or signal changes detected from the optically (e.g., fluorescently) labeled nucleotides may be indicative of consecutive nucleotides having incorporated into the plurality of growing nucleic acid strands. Methods for determining a number of fluorophores from the detected signals or signal changes are described elsewhere herein.

Alternatively, the optically (e.g., fluorescently) labeled nucleotides may be terminated. In such cases, each growing strand may incorporate no more than one nucleotide per flow cycle until synthesis is terminated. The one or more signals or signal changes detected from the optically (e.g., fluorescently) labeled nucleotides may be indicative of nucleotides having incorporated into the plurality of growing nucleic acid strands. Prior to, during, or subsequent to detection, a terminating group of the labeled nucleotides may be cleaved (e.g., to facilitate sequencing of homopolymers, and/or to reduce potential context and/or quenching issues).

Alternatively or in addition to, the optically (e.g., fluorescently) labeled nucleotides may include a mixture of terminated and non-terminated nucleotides. In such cases, the growing strands may incorporate one or more consecutive nucleotides generating an extended primer. The solution comprising the plurality of terminated and non-terminated nucleotides may then be washed away from the sequencing template. Un-labeled nucleotides of the plurality of nucleotides may comprise nucleotide moieties of the same type as labeled nucleotides of the plurality of nucleotides (e.g., the same canonical nucleotide).

In an aspect, the present disclosure provides compositions comprising one or more fluorescently labeled nucleotides and methods of using the same. A composition may comprise a solution comprising a fluorescently labeled nucleotide (e.g., as described herein). The fluorescently labeled nucleotide may comprise a fluorescent dye that is connected to a nucleotide or nucleotide analog (e.g., as described herein) via a linker (e.g., as described herein). The linker may comprise (i) one or more water soluble groups and (ii) two or more ring systems. At least two of the two or more ring systems may be connected to each other by no more than two sp³ carbon atoms, such as by no sp³ carbon atoms. For example, at least two of the two or more ring systems may be connected to each other by no more than two atoms. For example, at least two of the two or more ring systems may be connected to each other by an sp² carbon atom. The linker may comprise a non-proteinogenic amino acid comprising a ring system of the two or more ring systems. The fluorescently labeled nucleotide may be configured to emit a fluorescent signal. The fluorescently labeled nucleotide may comprise a plurality of amino acids, such as a plurality of non-proteinogenic (e.g., non-natural) amino acids. For example, the linker may comprise a plurality of hydroxyprolines. At least one water-soluble group of the one or more water-soluble groups may be appended to a ring structure of the two or more ring systems. The one or more water soluble groups may be selected from the group consisting of a pyridinium, an imidazolium, a quaternary ammonium group, a sulfonate, a phosphate, an alcohol, an amine, an imine, a nitrile, an amide, a thiol, a carboxylic acid, a polyether, an aldehyde, a boronic acid, and a boronic ester. The linker may comprise a cleavable group (e.g., an azidomethyl group, a disulfide bond, a hydrocarbyldithiomethyl group, and a 2-nitrobenzyloxy group) that is configured to be cleaved to separate the fluorescent dye from the nucleotide.

The solution (e.g., nucleotide flow) may comprise a plurality of fluorescently labeled nucleotides, each or which may comprise a fluorescent dye of a same type, a linker of a same type, and a nucleotide of a same type. Each linker of each fluorescently labeled nucleotide of the plurality of fluorescently labeled nucleotides may have the same molecular weight (e.g., they might not comprise polymers with a range of molecular weights). The solution may also comprise a plurality of unlabeled nucleotides, in which each nucleotide of the plurality of unlabeled nucleotides is of a same type as each nucleotide of the plurality of fluorescently labeled nucleotides. The ratio of the plurality of fluorescently labeled nucleotides to the plurality of unlabeled nucleotides in the solution may be at least about 1:4 (e.g., the labeling fraction may be at least 20%). For example, the ratio may be at least 1:1 (e.g., the labeling fraction may be at least 50%). Alternatively, the solution may not comprise any unlabeled nucleotides and the labeling fraction may be 100%.

The solution (e.g., nucleotide flow) may be provided to a template nucleic acid molecule coupled to a nucleic acid strand. The template nucleic acid molecule may be immobilized to a support (e.g., as described herein). For example, the template nucleic acid molecule may be immobilized to a support via an adapter. For example, the template nucleic acid molecule may be immobilized to a support via a primer to which it is hybridized. The nucleic acid strand may be at least partially complementary to a portion of the template nucleic acid molecule. The template nucleic acid molecule and nucleic acid strand coupled thereto may be subjected to conditions sufficient to incorporate a fluorescently labeled nucleotide of the solution into the nucleic acid strand coupled to the template nucleic acid molecule. Incorporation of the fluorescently labeled nucleotide may be accomplished using a polymerase enzyme (e.g., as described herein). More than one fluorescently labeled nucleotide of the solution may be incorporated, such as into a homopolymeric region of the template nucleic acid molecule. Alternatively or in addition to, an unlabeled nucleotide may be incorporated (e.g., adjacent to the fluorescently labeled nucleotide), such as into a homopolymeric region of the template nucleic acid molecule. A signal (e.g., a fluorescent signal) may be detected from the fluorescently labeled nucleotide incorporated into the nucleic acid strand. Prior to detection of the signal, a wash solution may be used to remove fluorescently labeled nucleotides that are not incorporated into the nucleic acid strand. After detection of the signal, the fluorescently labeled nucleotide incorporated into the nucleic acid strand may be contacted with a cleavage reagent configured to cleave the fluorescent dye from the nucleotide. The cleavage reagent may be configured to cleave the linker to provide the nucleotide attached to a portion of the linker, which portion may comprise a thiol moiety, an aromatic moiety, or a combination thereof. The nucleic acid strand, such as a nucleic acid strand of a plurality of nucleic acid strands coupled to a plurality of template nucleic acid molecules, may be contacted with a chase flow comprising only unlabeled nucleotides of a same nucleotide type (e.g., before or after detection of a signal). The nucleic acid strand coupled to the template nucleic acid molecule may also be contacted with one or more additional wash flows. The nucleic acid strand coupled to the template nucleic acid molecule may be contacted with an additional solution comprising an additional fluorescently labeled nucleotide, such as an additional fluorescently labeled nucleotide including a nucleotide of a different type. The dye of the additional fluorescently labeled nucleotide may be of a same type as the dye of the fluorescently labeled nucleotide. Similarly, the linker of the additional fluorescently labeled nucleotide may be of a same type as the linker of the fluorescently labeled nucleotide.

In another aspect, the present disclosure provides a method comprising providing a fluorescent labeling reagent (e.g., as described herein). The fluorescent labeling reagent may comprise a fluorescent dye and a linker that is connected to the fluorescent dye. The linker may comprise (i) one or more water soluble groups and (ii) two or more ring systems. At least two of the two or more ring systems may be connected to each other by no more than two sp³ carbon atoms, such as by no more than two atoms. For example, at least two of the two or more ring structures may be connected to each other by an sp² carbon atom. The linker may comprise a non-proteinogenic amino acid comprising a ring system of the two or more ring systems. The fluorescent labeling reagent may be configured to emit a fluorescent signal. The fluorescent labeling reagent may comprise a plurality of amino acids, such as a plurality of non-proteinogenic (e.g., non-natural) amino acids. For example, the linker may comprise a plurality of hydroxyprolines. At least one water-soluble group of the one or more water-soluble groups may be appended to a ring structure of the two or more ring systems. The one or more water soluble groups may be selected from the group consisting of a pyridinium, an imidazolium, a quaternary ammonium group, a sulfonate, a phosphate, an alcohol, an amine, an imine, a nitrile, an amide, a thiol, a carboxylic acid, a polyether, an aldehyde, a boronic acid, and a boronic ester.

A substrate may be contacted with the fluorescent labeling reagent to generate a fluorescently labeled substrate, in which the linker connected to the fluorescent dye is associated with the substrate. The substrate may be a nucleotide or nucleotide analog (e.g., as described herein). Alternatively, the substrate may be a protein, lipid, cell, or antibody. The fluorescently labeled substrate may be configured to emit a fluorescent signal (e.g., upon excitation at an appropriate energy range), which signal may be detected (e.g., using imaging-based detection). The linker may comprise a cleavable group (e.g., an azidomethyl group, a disulfide bond, a hydrocarbyldithiomethyl group, and a 2-nitrobenzyloxy group) that is configured to be cleaved to separate the fluorescent dye from the substrate. The fluorescently labeled substrate may be contacted with a cleavage reagent configured to cleave the fluorescent labeling reagent or a portion thereof from the fluorescently labeled substrate to generate a scarred substrate. The scarred substrate may comprise a thiol moiety, a propargyl moiety, an olefin moiety, a hydroxyl moiety, an amine moiety, an aromatic moiety, or a combination thereof. A scarred substrate may comprise an aromatic moiety such as a phenyl or benzyl moiety. Prior to generating the scarred substrate, the fluorescently labeled substrate and a nucleic acid molecule may be subjected to conditions sufficient to incorporate the fluorescently labeled substrate into the nucleic acid molecule. Incorporation may be accomplished using a polymerase enzyme (e.g., as described herein). More than one fluorescently labeled substrate may be incorporated, such as into a homopolymeric region of the nucleic acid molecule. For example, an additional fluorescently labeled substrate may be incorporated into a position adjacent to the position into which the fluorescently labeled substrate is incorporated. Alternatively or in addition to, an unlabeled substrate (e.g., a nucleotide of a same type as the nucleotide of a fluorescently labeled nucleotide) may also be incorporated into the nucleic acid molecule, such as into adjacent positions of the nucleic acid molecule. Incorporation of an additional fluorescently labeled substrate may occur before or after generation of the scarred substrate. Similarly, incorporation of an unlabeled substrate may occur before or after generation of the scarred substrate.

The nucleic acid molecule, such as a nucleic acid molecule of a plurality of nucleic acid molecules, may be contacted with a chase flow comprising only unlabeled substrates of a same type (e.g., before or after detection of a signal from the nucleic acid molecule). The nucleic acid molecule may also be contacted with one or more additional wash flows. The nucleic acid molecule may be contacted with an additional solution comprising an additional fluorescently labeled substrate, such as an additional fluorescently labeled substrate including a nucleotide of a different type. The dye of the additional fluorescently labeled substrate may be of a same type as the dye of the fluorescently labeled substrate. Similarly, the linker of the additional fluorescently labeled substrate may be of a same type as the linker of the fluorescently labeled substrate.

The nucleic acid molecule may be immobilized to a support (e.g., as described herein). For example, the nucleic acid molecule may be immobilized to a support via an adapter. For example, the nucleic acid molecule may be immobilized to a support via a primer to which it is hybridized. The nucleic acid molecule may comprise a first nucleic acid strand that is at least partially complementary to a portion of a second nucleic acid strand. The second nucleic acid strand may comprise a template nucleic acid sequence, or a complement thereof.

The labeled nucleotides of the present disclosure may be used during sequencing operations that involve a high fraction of labeled nucleotides. For example, the present disclosure provides a method comprising contacting a nucleic acid molecule (e.g., a template nucleic acid molecule) with a solution comprising a plurality of nucleotides under conditions sufficient to incorporate a first labeled nucleotide and a second labeled nucleotide of the plurality of nucleotides into a growing strand that is at least partially complementary to the nucleic acid molecule. The first labeled nucleotide and the second labeled nucleotide may be of a same canonical base type. The first nucleotide may comprise a fluorescent dye (e.g., as described herein), which fluorescent dye may be associated with the first nucleotide via a linker (e.g., as described herein). The second nucleotide may comprise the same fluorescent dye (e.g., associated with the second nucleotide via a linker having the same chemical structure of the linker associating the first nucleotide and the fluorescent dye). A fluorescent dye coupled to a nucleotide (e.g., the first and/or second nucleotide) may be cleavable (e.g., upon application of a cleavage reagent). At least about 20% of the plurality of nucleotides may be labeled nucleotides. For example, at least 20% of the plurality of nucleotides may be associated with a fluorescent labeling reagent (e.g., as described herein). For example, at least about 50%, 70%, 80%, 90%, 95%, or 99% of the plurality of nucleotides may be labeled nucleotides. For example, all of the nucleotides of the plurality of nucleotides may be labeled nucleotides (e.g., the labeling fraction may be 100%). One or more signals or signal changes may be detected from the first labeled nucleotide and the second labeled nucleotide (e.g., as described herein). The one or more signals or signal changes may comprise fluorescent signals or signal changes. The one or more signals or signal changes may be indicative of incorporation of the first labeled nucleotide and the second labeled nucleotide. The one or more signals or signal changes may be resolved to determine a sequence of the nucleic acid molecule, or a portion thereof. Resolving the one or more signals or signal changes may comprise determining a number of consecutive nucleotides from the solution that incorporated into the growing strand. The number of consecutive nucleotides may be selected from the group consisting of 2, 3, 4, 5, 6, 7, or 8 nucleotides. Resolving the one or more signals or signal changes may comprise processing a tolerance of the solution. A third nucleotide may also be incorporated into the growing strand (e.g., before or after detection of the one or more signals or signal changes). The third nucleotide may be a nucleotide of the plurality of nucleotides of the solution. Alternatively, the third nucleotide may be provided in a separate solution, such as in a “chase” flow (e.g., as described herein). The third nucleotide may be unlabeled. Alternatively, the third nucleotide may be labeled. The first labeled nucleotide and the third nucleotide may be of a same canonical base type. Alternatively, the first labeled nucleotide and the third nucleotide may be of different canonical base types.

The method may further comprise cleaving the fluorescent dye coupled to the first labeled nucleotide. The fluorescent dye may be cleaved by application of a cleavage reagent configured to cleave a linker associating the first labeled nucleotide and the fluorescent dye. The nucleic acid molecule may be contacted with a second solution comprising a second plurality of nucleotides under conditions sufficient to incorporate a third labeled nucleotide of the second plurality of nucleotides into the growing strand. At least about 20% of the second plurality of nucleotides may be labeled nucleotides (e.g., as described herein). One or more second signals or signal changes may be detected from the third labeled nucleotide (e.g., as described herein). The one or more second signals or signal changes may be resolved to determine a second sequence of the nucleic acid molecule, or a portion thereof. The first labeled nucleotide and the third labeled nucleotide may be different canonical base types (e.g., A, C, U/T, or G). The third labeled nucleotide may comprise the fluorescent dye. The fluorescent dye may be coupled to the third labeled nucleotide via a linker (e.g., as described herein), which linker may have the same chemical structure as the linker connecting the fluorescent dye to the first labeled nucleotide or a different chemical structure.

Alternatively, the method may comprise contacting the nucleic acid molecule with a second solution comprising a second plurality of nucleotides under conditions sufficient to incorporate a third labeled nucleotide of the second plurality of nucleotides into the growing strand. At least about 20% of the second plurality of nucleotides may be labeled nucleotides (e.g., as described herein). One or more second signals or signal changes may be detected from the third labeled nucleotide (e.g., as described herein). The one or more second signals or signal changes may be resolved to determine a second sequence of the nucleic acid molecule, or a portion thereof. The first labeled nucleotide and the third labeled nucleotide may be different canonical base types (e.g., A, C, U/T, or G). The third labeled nucleotide may comprise the fluorescent dye. The fluorescent dye may be coupled to the third labeled nucleotide via a linker (e.g., as described herein), which linker may have the same chemical structure as the linker connecting the fluorescent dye to the first labeled nucleotide or a different chemical structure. Contacting the nucleic acid molecule with the second solution may be performed in absence of cleaving a fluorescent dye from the first labeled nucleotide or the second labeled nucleotide. This process may be repeated one or more times, such as 1, 2, 3, 4, 5, or more times, each with a different solution of nucleotides, in absence of cleaving a fluorescent dye from the first labeled nucleotide or the second labeled nucleotide. One or more of these different solutions of nucleotides may comprise at least 20% labeled nucleotides.

The present disclosure also provides a method comprising contacting a nucleic acid molecule with a solution comprising a plurality of non-terminated nucleotides under conditions sufficient to incorporate a labeled nucleotide and a second nucleotide of the plurality of non-terminated nucleotides into a growing strand that is at least partly complementary to the nucleic acid molecule, or a portion thereof. The labeled nucleotide and the second nucleotide may be of a same canonical base type. Alternatively, the labeled nucleotide and the second nucleotide may be of different canonical base types. The labeled nucleotide may comprise a fluorescent dye (e.g., as described herein), which fluorescent dye may be associated with the labeled nucleotide via a linker (e.g., as described herein). The second nucleotide may be a labeled nucleotide. For example, the second nucleotide may comprise the same fluorescent dye (e.g., associated with the second nucleotide via a linker having the same chemical structure of the linker associating the first nucleotide and the fluorescent dye). Alternatively, the second nucleotide may not be coupled to a fluorescent dye (e.g., the second nucleotide may be unlabeled). A fluorescent dye coupled to a nucleotide (e.g., the first and/or second nucleotide) may be cleavable (e.g., upon application of a cleavage reagent). The plurality of non-terminated nucleotides may comprise nucleotides of a same canonical base type. At least about 20% of said plurality of nucleotides may be labeled nucleotides. For example, at least 20% of the plurality of nucleotides may be associated with a fluorescent labeling reagent (e.g., as described herein). For example, at least about 50%, 70%, 80%, 90%, 95%, or 99% of the plurality of non-terminated nucleotides may be labeled nucleotides. For example, substantially all of the plurality of non-terminated nucleotides may be labeled nucleotides. For example, all of the nucleotides of the plurality of non-terminated nucleotides may be labeled nucleotides (e.g., the labeling fraction may be 100%). One or more signals or signal changes may be detected from the labeled nucleotide (e.g., as described herein). The one or more signals or signal changes may comprise fluorescent signals or signal changes. The one or more signals or signal changes may be indicative of incorporation of the labeled nucleotide. The one or more signals or signal changes may be resolved to determine a sequence of the nucleic acid molecule, or a portion thereof. Resolving the one or more signals or signal changes may comprise determining a number of consecutive nucleotides from the solution that incorporated into the growing strand. The number of consecutive nucleotides may be selected from the group consisting of 2, 3, 4, 5, 6, 7, or 8 nucleotides. Resolving the one or more signals or signal changes may comprise processing a tolerance of the solution. A third nucleotide may also be incorporated into the growing strand (e.g., before or after detection of the one or more signals or signal changes). The third nucleotide may be a nucleotide of the plurality of non-terminated nucleotides of the solution. Alternatively, the third nucleotide may be provided in a separate solution, such as in a “chase” flow (e.g., as described herein). The third nucleotide may be unlabeled. Alternatively, the third nucleotide may be labeled. The labeled nucleotide and the third nucleotide may be of a same canonical base type. Alternatively, the labeled nucleotide and the third nucleotide may be of different canonical base types.

The method may further comprise cleaving the fluorescent dye coupled to the labeled nucleotide. The fluorescent dye may be cleaved by application of a cleavage reagent configured to cleave a linker associating the labeled nucleotide and the fluorescent dye. The nucleic acid molecule may be contacted with a second solution comprising a second plurality of non-terminated nucleotides under conditions sufficient to incorporate a third labeled nucleotide of the second plurality of non-terminated nucleotides into the growing strand. At least about 20% of the second plurality of non-terminated nucleotides may be labeled nucleotides (e.g., as described herein). One or more second signals or signal changes may be detected from the third labeled nucleotide (e.g., as described herein). The one or more second signals or signal changes may be resolved to determine a second sequence of the nucleic acid molecule, or a portion thereof. The first labeled nucleotide and the third labeled nucleotide may be different canonical base types (e.g., A, C, U/T, or G). The third labeled nucleotide may comprise the fluorescent dye. The fluorescent dye may be coupled to the third labeled nucleotide via a linker (e.g., as described herein), which linker may have the same chemical structure as the linker connecting the fluorescent dye to the first labeled nucleotide or a different chemical structure.

Alternatively, the method may comprise contacting the nucleic acid molecule with a second solution comprising a second plurality of non-terminated nucleotides under conditions sufficient to incorporate a third labeled nucleotide of the second plurality of non-terminated nucleotides into the growing strand. At least about 20% of the second plurality of nucleotides may be labeled nucleotides (e.g., as described herein). One or more second signals or signal changes may be detected from the third labeled nucleotide (e.g., as described herein). The one or more second signals or signal changes may be resolved to determine a second sequence of the nucleic acid molecule, or a portion thereof. The first labeled nucleotide and the third labeled nucleotide may be different canonical base types (e.g., A, C, U/T, or G). The third labeled nucleotide may comprise the fluorescent dye. The fluorescent dye may be coupled to the third labeled nucleotide via a linker (e.g., as described herein), which linker may have the same chemical structure as the linker connecting the fluorescent dye to the first labeled nucleotide or a different chemical structure. Contacting the nucleic acid molecule with the second solution may be performed in absence of cleaving a fluorescent dye from the first labeled nucleotide or the second labeled nucleotide. This process may be repeated one or more times, such as 1, 2, 3, 4, 5, or more times, each with a different solution of nucleotides, in absence of cleaving a fluorescent dye from the first labeled nucleotide or the second labeled nucleotide. One or more of these different solutions of nucleotides may comprise at least 20% labeled nucleotides.

In an aspect, the present disclosure provides a method for identifying a nucleotide or nucleotide sequence of a nucleic acid using a cleavable, immolating linker. Dye- and linker-bound nucleotides can present a number of challenges to sequencing methods. In some cases, a dye, linker, or scar in a nucleic acid molecule may increase the frequency of mispair incorporations into the nucleic acid molecule or inhibit the rate of subsequent nucleotide incorporations. These challenges can be overcome by the use of linkers that form benign immolated scars upon cleavage.

The method may comprise contacting the nucleic acid molecule with a first nucleotide coupled to a cleavable linker comprising a first detectable moiety, detecting the detectable moiety, cleaving the linker, and contacting the nucleic acid molecule with a second nucleotide. The first nucleotide and second nucleotide may be provided as pluralities of nucleotides containing only labeled nucleotides, or as pluralities of nucleotides containing a mixture of labeled and unlabeled nucleotides. The second nucleotide may be provided as a plurality of nucleotides, for example containing only unlabeled nucleotides, only labeled nucleotides, or a mixture of labeled and unlabeled nucleotides. The plurality of first or second nucleotides may comprise a single canonical type of nucleotide (e.g., only adenosine and functionalized derivatives thereof), or multiple types of nucleotides (e.g., A, G, C, and U/T). The nucleic acid molecule may be contacted to a polymerase under conditions permissive for nucleotide incorporation into the nucleic acid (e.g., conditions in which the polymerase comprises activity and the nucleic acid molecule is hybridized). The detectable moiety may comprise an optically detectable moiety. The optically detectable moiety may comprise a dye (e.g., a fluorescent dye). The detecting may comprise fluorescence detection. The detection may comprise imaging (e.g., fluorescence imaging).

In some cases, the first nucleotide may be provided as a first plurality of nucleotides of a single canonical base type, that are non-terminated, such that during the first flow in which the nucleic acid molecule is contacted with the first plurality of nucleotides, if an open position in the growing nucleic acid strand is part of a homopolymer region, multiple nucleotides of the first plurality of nucleotides (including the first nucleotide) are incorporated consecutively during the first flow. The detectable moiety of at least the first nucleotide may be detected, and the linker cleaved. Then, the nucleic acid molecule may be contacted with the second nucleotide in a subsequent flow. In between the first flow and the subsequent flow, there may be one or more intermediate flows, each intermediate flow comprising a plurality of nucleotides comprising nucleotides of a single canonical base type, which are entirely unlabeled. The cleavage reaction may occur at any point subsequent to detection of the detectable moiety and incorporation of the second nucleotide, such as in between the first flow (containing the first nucleotide) and an intermediary flow, during an intermediary flow, or in between an intermediary flow and the subsequent flow (containing the second nucleotide).

The method may comprise cleaving the linker. In some cases, the cleaving comprises contacting the linker with a reducing agent. In some cases, the reducing reagent comprises one or more members selected from the group consisting of: tetrahydropyran, β-mercaptoethanol (β-ME), dithiothreitol (DTT), tris(2-carboxyethyl)phosphine (TCEP), Ellman's reagent, hydroxylamine, and cyanoborohydride. The cleaving may decouple the detectable moiety from the first nucleotide. The cleaving may generate a scar on the first nucleotide. The scar may undergo an immolation reaction. The immolation reaction may be spontaneous, or may occur upon irradiation, a change in conditions (e.g., a change in pH or temperature), or upon contact with a reagent, such as a catalyst (e.g., an enzyme). The scar or immolated scar may comprise a primary amine or a primary hydroxyl. The scar or immolated scar may terminate in a primary amine or primary hydroxyl. The scar or immolated scar may comprise a propargyl amine or a propargyl alcohol. The scar or immolated scar may be a propargyl amine or a propargyl alcohol.

For cases in which the first and second nucleotide incorporate into the nucleic acid molecule, the cleavage of the linker coupled to the first nucleotide may enhance the rate of incorporation of the second nucleotide into the primer. While scarred nucleotides may inhibit the rates of subsequent nucleic acid incorporations, an immolated scar may be less inhibitory than a non-immolated scar. For example, a linker comprising a cleavable disulfide adjacent to a carbamate may immolate to yield an amine or hydroxyl scar, while a linker comprising a cleavable disulfide adjacent to an amide or ester may not immolate following disulfide cleavage, and therefore may yield a thiol scar. In such a case, the immolated amine or hydroxyl scar may be less inhibitory towards nucleic acid polymerization than the non-immolated thiol scar. In some cases, the rate of incorporation of the second nucleotide is at least 0.0001 per second (s⁻¹), at least 0.0005 s⁻¹, at least 0.001 s⁻¹, at least 0.005 s⁻¹, at least 0.01 s⁻¹, at least 0.05 s⁻¹, at least 0.1 s⁻¹, or at least 0.5 s⁻¹. In some cases, the rate of incorporation of the second nucleotide is at least 0.1%, at least 0.2%, at least 0.5%, at least 0.8%, at least 1%, at least 1.2%, at least 1.5%, at least 2%, at least 2.5%, at least 3%, at least 4%, at least 5%, at least 6%, at least 8%, at least 10%, at least 12%, at least 15%, at least 18%, at least 20%, at least 25%, at least 30%, at least 40%, at least 50%, at least 60%, at least 70%, or at least 80% of a rate of incorporation of said second nucleotide incorporating adjacent to a nucleotide (i) of a same canonical type as said first nucleotide and (ii) that lacks said primary amine or primary hydroxyl moiety.

Furthermore, the cleavage of the linker coupled to the first nucleotide may diminish the misincorporation rate at a position adjacent to the first nucleotide. The incorporation of said second nucleotide may comprise a misincorporation rate of less than 20%. The incorporation of said second nucleotide may comprise a misincorporation rate of less than 15%. The incorporation of said second nucleotide may comprise a misincorporation rate of less than 10%. The incorporation of said second nucleotide may comprise a misincorporation rate of less than 8%. The incorporation of said second nucleotide may comprise a misincorporation rate of less than 5%. The incorporation of said second nucleotide may comprise a misincorporation rate of less than 4%. The incorporation of said second nucleotide may comprise a misincorporation rate of less than 3%. The incorporation of said second nucleotide may comprise a misincorporation rate of less than 2%. The incorporation of said second nucleotide may comprise a misincorporation rate of less than 1%. The incorporation of said second nucleotide may comprise a misincorporation rate of less than 0.5%. The incorporation of said second nucleotide may comprise a misincorporation rate of less than 0.1%.

### Methods for Constructing Labeled Nucleotides

In an aspect, the present disclosure provides methods for constructing labeled nucleotides (e.g., optically labeled nucleotides). Labeled nucleotides can be constructed using modular chemical building blocks. A nucleotide or nucleotide analog can be derivatized with, e.g., a propargylamino moiety to provide a handle for attachment to a linker or detectable label (e.g., dye). One or more detectable labels, such as one or more dyes, can be attached to a nucleotide or nucleotide analog via a covalent bond. Alternatively or in addition to, one or more detectable labels can be attached to a nucleotide or nucleotide analog via a non-covalent bond. A detectable label may be attached to a nucleotide or nucleotide analog via a linker (e.g., as described herein). A linker may be configured to couple to a nucleotide or nucleotide analogue. A linker may be configured to couple to a nucleobase of the nucleotide or nucleotide analogue. A linker may include one or more moieties. For example, a linker may include a first moiety including a disulfide bond within it to facilitate cleaving the linker and releasing the detectable label (e.g., during a sequencing process). Additional linker moieties can be added using sequential peptide bonds. Linker moieties can have various lengths and charges. A linker moiety may include one or more different components, such as one or more different ring systems, and/or a repeating unit (e.g., as described herein). Examples of linkers include, but are not limited to, aminoethyl-SS-propionic acid (epSS), aminoethyl-SS-benzoic acid, aminohexyl-SS-propionic acid, hyp10, and hyp20.

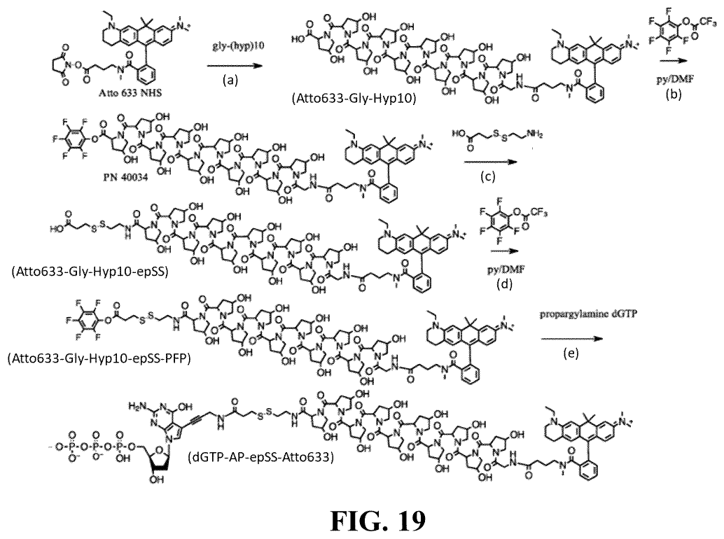

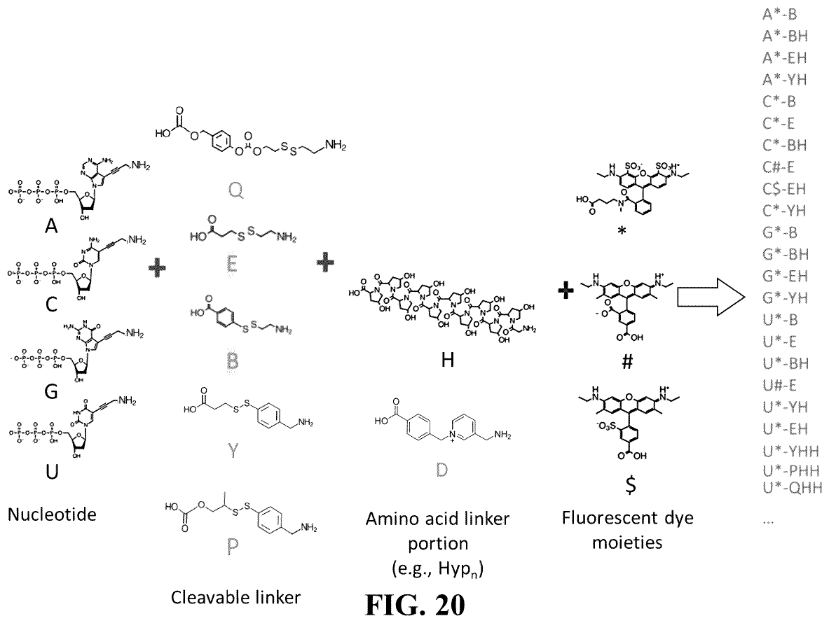

Examples of methods for constructing labeled nucleotides are shown in FIGS. 19 and 20. As shown in FIG. 20, a labeled nucleotide may be constructed from a nucleotide, a dye, and one or more linker moieties. The one or more linker moieties together comprise a linker as described herein. A nucleotide functionalized with a propargylamino moiety can be attached to a first linker moiety via a peptide bond. This first linker moiety may comprise a cleavable moiety, such as a disulfide moiety. The first linker moiety can also be attached to one or more additional linker moieties in linear or branching fashions. For example, a second linker moiety may include two or more ring systems, wherein at least two of the two or more ring systems are separated by no more than two sp3 carbon atoms, such as by no more than two atoms. For example, at least two of the two or more ring systems may be connected to each other by an sp² carbon atom. The linker may comprise a non-proteinogenic amino acid comprising a ring system of the two or more ring systems. For example, the second linker moiety may comprise a two or more hydroxyproline moieties. An amine handle on a linker moiety may be used to attach the linker and a dye, such as a dye that fluoresces in the red or green portions of the visible electromagnetic spectrum. The labeled nucleotide generated in FIG. 20 comprises a modified deoxyadenosine triphosphate moiety, a linker comprising a first linker moiety including a disulfide moiety and a second linker moiety including at least two ring systems, and a dye.

Construction of a labeled nucleotide can begin from either the nucleotide terminus or the dye terminus. Construction from the dye terminus permits the use of unlabeled, unactivated amino acid moieties, while construction from the nucleotide terminus may require amine-protected, carboxy-activated amino acid moieties.

A nucleotide or nucleotide analog of a labeled nucleotide may include one or more modifications, such as one or more modifications on the nucleobase. Alternatively, a nucleotide or nucleotide analog of a labeled nucleotide may include one or more modifications not on the nucleobase. Modifications can include, but are not limited to, covalent attachment of one or more linker or label moieties, alkylation, amination, amidation, esterification, hydroxylation, halogenation, sulfurylation, and/or phosphorylation.

A nucleotide or nucleotide analog of a labeled nucleotide may include one or more modifications that are configured prevent subsequent nucleotide additions to a position adjacent to the labeled nucleotide upon its incorporation into a growing nucleic acid strand. For example, the labeled nucleotide may include a terminating or blocking group (e.g., dimethoxytrityl, phosphoramidite, or nitrobenzyl molecules). In some instances, the terminating or blocking group may be cleavable.

### Capping Reagents

Various aspects of the present disclosure provide reagents, compositions, and methods for capping scar moieties. Linker cleavage can generate a residual scar group that affects a property (e.g., pKa or polarity) of the substrate to which it is bound. In some cases, a scar may inhibit a rate or the accuracy of an enzymatic process, such as nucleic acid polymerization. For example, a scar group disposed on a 3′ end of a nucleic acid molecule may inhibit rates of subsequent nucleotide incorporations into the nucleic acid. A property (e.g., pKa) or an adverse effect (e.g., polymerase inhibition) of a scar group may be altered by coupling the scar group to a capping reagent.

A method utilizing capping reagents may be optimized to prevent non-scar moiety capping. A capping reagent that undesirably or promiscuously reacts may adversely affect an assay by inhibiting an enzymatic process (e.g., nucleic acid polymerization), by decreasing the accuracy of the process (e.g., by increasing mispairing during nucleic acid polymerization), or by passivating reagents (e.g., by coupling to nucleoside triphosphate reagents so as to inhibit their incorporation into nucleic acid molecules). The present disclosure provides capping reagents which comprise higher reactivity towards scars than toward non-scar portions of nucleic acids (e.g., nucleobase, sugar, and triphosphate moieties), proteins (e.g., polymerases), and optional nucleic acid polymerization reagents (e.g., nucleoside triphosphates and Mg²⁺).

The capping reagent may covalently (e.g., for a bond with) or non-covalently couple to the scar group. A capping reagent may covalently couple to a nucleophilic moiety on a scar, such as a hydroxyl or thiol. A capping reagent may reversibly or irreversibly couple to a scar. Examples of reversibly-binding capping reagents (“reversible capping reagents) include

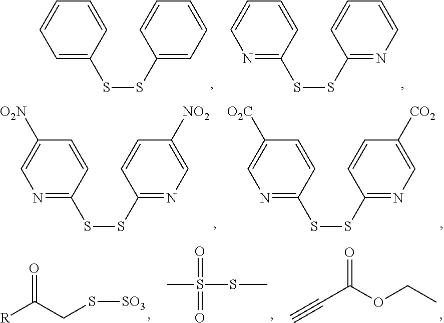

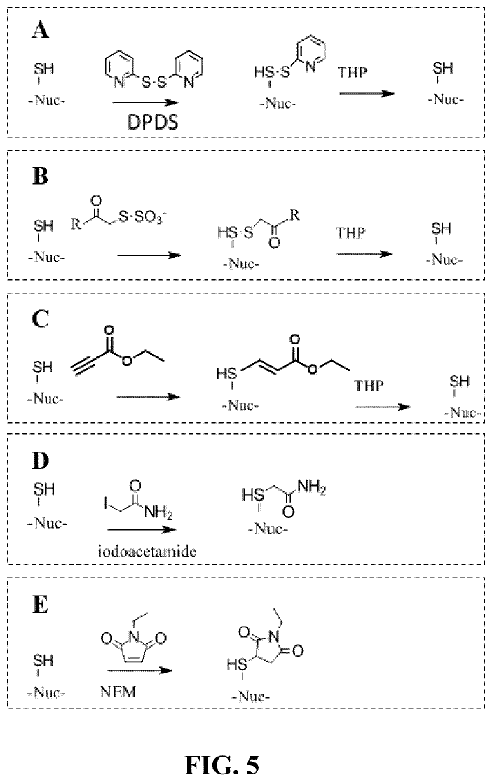

and optionally substituted (e.g., alkylated, halogenated, or carboxylated) variants thereof. A reversible thiol capping reagent may comprise a disulfide, a thiosulfate, or an alkyne, and may cap a thiol scar through a thiol-disulfide exchange, as is illustrated in FIG. 5 Panels A-B, or a thiol-yne reaction, as is illustrated in FIG. 5 Panel C. Reversible capping of a thiol scar may convert the thiol into a disulfide. The disulfide may subsequently be cleaved by a reducing agent, such as THP. In some cases, a single reagent may cleave a cleavable linker and remove a reversible capping reagent. For example, a reducing reagent such as THP may remove a thiolate (e.g., a pyridine thiolate derived from a dipyridyldisulfide capping reagent or a benzenethiolate derived from a dibenzyldisulfide capping reagent).

A capping reagent or a portion thereof (e.g., a methyl or acetyl group) may irreversibly couple to a scar. In some cases, irreversible coupling denotes formation of a stable bond in the conditions of and upon contact with the reagents for a particular assay. For example, a hydroxyl scar methylating reagent may be an irreversible capping reagent in a nucleic acid polymerization assay if none of the conditions or reagents of the assay are configured to remove a methyl group from a methoxide moiety. An irreversible thiol capping reagent may comprise an iodoacetyl or pyrrole dione moiety. Examples of irreversible thiol capping reagents include

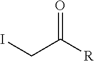

(wherein R may comprise O, S, optionally substituted alkyl, optionally substituted alkenyl, optionally substituted alkynyl, optionally substituted amine, optionally substituted alkoxide, cycloalkyl, optionally substituted heterocycloalkyl, optionally substituted aryl, and optionally substituted heteroaryl),

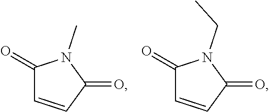

and optionally substituted (e.g., alkylated, halogenated, or carboxylated) variants thereof. An irreversible thiol capping reagent may comprise a substitutable halogen (e.g., iodide in iodoacetamide) or an electrophilic olefin (e.g., the double bonded carbons of a pyrrole dione), and may form a carbon-sulfur bond between the thiol scar and the capping reagent or a portion thereof, as is illustrated in FIG. 5 Panels D and E.

Aspects of the present disclosure provide methods for sequencing nucleic acids with labeled nucleotides (e.g., nucleoside triphosphates) and capping reagents. A method may comprise incorporating a labeled nucleotide into a nucleic acid strand, contacting the labeled nucleotide with a capping reagent to generate a capped moiety, and incorporating an additional nucleotide adjacent to the labeled nucleotide. The nucleic acid strand may be hybridized to a nucleic acid molecule, such as a template strand. The labeled nucleotide may be a terminated nucleotide (e.g., may comprise a 3′-capped deoxyribosyl moiety that prevents subsequent nucleotide incorporations). In such cases, the labeled nucleotide may be converted to a non-terminated nucleotide (e.g., may be contacted with a reagent that removes a 3′-cap). For example, the method may comprise incorporating the labeled nucleotide into the nucleic acid strand, wherein the labeled nucleotide is a terminated nucleotide, detecting the labeled nucleotide, converting the labeled nucleotide into a non-terminated nucleotide, and incorporating the additional nucleotide adjacent to the labeled nucleotide. In some cases, the additional nucleotide may be a terminated nucleotide. The labeled nucleotide and the additional nucleotide may each be non-terminated.

Incorporation of the labeled nucleotide may comprise contacting the nucleic acid strand with a plurality of nucleotides. The plurality of nucleotides may comprise a single type of canonical nucleotide. The plurality of nucleotides may also comprise more than one type of canonical nucleotide. In such cases, a labeled nucleotide of a first canonical type may comprise a different detectable label than a labeled nucleotide of a second canonical type. For example, a plurality of nucleotides may comprise an adenosine triphosphate coupled to a blue dye (e.g., ATTO 390) and a cytidine triphosphate coupled to a red dye (e.g., ATTO 590). The plurality of nucleotides may comprise a mixture of labeled and unlabeled nucleotides (e.g., the plurality of nucleotides may consist of 80% labeled adenine nucleoside triphosphates and 20% unlabeled adenosine triphosphates). The ratio of labeled to unlabeled nucleotides may be at least 1:40 (labeled to unlabeled nucleotides), at least 1:30, at least 1:25, at least 1:20, at least 1:15, at least 1:10, at least 1:8, at least 1:5, at least 1:4, at least 1:3, at least 2:5, at least 1:2, at least 2:3, at least 1:1, at least 3:2, at least 2:1, at least 5:2, at least 3:1, at least 4:1, at least 5:1, at least 8:1, at least 10:1, at least 15:1, at least 20:1, at least 25:1, or at least 30:1. The ratio of labeled to unlabeled nucleotides may be at most 1:40, at most 1:30, at most 1:25, at most 1:20, at most 1:15, at most 1:10, at most 1:8, at most 1:5, at most 1:4, at most 1:3, at most 2:5, at most 1:2, at most 2:3, at most 1:1, at most 3:2, at most 2:1, at most 5:2, at most 3:1, at most 4:1, at most 5:1, at most 8:1, at most 10:1, at most 15:1, at most 20:1, at most 25:1, at most 30:1, or at most 40:1. The incorporation of the labeled nucleotide may comprise incorporation of multiple nucleotides from among the plurality of nucleotides. For example, the nucleic acid strand may be hybridized to the nucleic acid molecule, and the nucleic acid molecule may comprise a homopolymeric region complementary to a canonical nucleotide type of the plurality of nucleotides. In some cases, the incorporation of multiple nucleotides comprises incorporation of 2 or more labeled nucleotides (e.g., coupled to linkers comprising detectable moieties). In such cases, the 2 or more labeled nucleotides may be detected, and the detection may identify the number of nucleotides incorporated into the nucleic acid strand. Linker cleavage may cleave linkers coupled to different canonical types of nucleotides or may only cleave a linker coupled to a specific type of canonical nucleotide. For example, the plurality of nucleotides may comprise a deoxyadenosine triphosphate coupled to a first linker configured for cleavage by a first reagent and a deoxyguanosine triphosphate coupled to a second linker configured for cleavage by a second reagent.

The incorporating the labeled nucleotide into the nucleic acid strand may identify a nucleotide of the nucleic acid molecule. For example, incorporation of a labeled guanosine into the nucleic acid strand may identify a cytosine in the nucleic acid molecule. The method may comprise a plurality of cycles, thereby enabling identification of multiple nucleotides in the nucleic acid molecule. For example, the method may comprise multiple labeled nucleotide incorporations, and may thereby identify a sequence or a portion of a sequence (e.g., 5 nucleotides within a 10 nucleotide sequence) of the nucleic acid molecule.

In some cases, the method may comprise cleaving a linker coupled to the labeled nucleotide, which may generate a scar moiety coupled to the labeled nucleotide. The linker may comprise a detectable moiety, such as a fluorescent dye. The method may comprise detecting the detectable moiety. The detection may be subsequent to the incorporation of the labeled nucleotide into the nucleic acid strand. The detection may be prior to the incorporation of the labeled nucleotide into the nucleic acid strand. For example, a polymerase may comprise second or minute timescale nucleotide incorporation rates, thereby enabling detection of the labeled nucleotide subsequent to polymerase binding but prior to the incorporation. In some cases, a method may include contacting the labeled nucleotide to the nucleic acid strand under conditions in which the polymerase lacks activity (e.g., in the presence of Ca²⁺), detecting the labeled nucleotide coupled to the nucleic acid strand and optionally bound to the polymerase, and then changing the conditions (e.g., washing out Ca²⁺ and adding Mg²⁺) to facilitate the incorporation of the labeled nucleotide into the nucleic acid strand. Cleavage of the linker may decouple the detectable moiety from the labeled nucleotide. The capping reagent may couple to the scar moiety coupled to the labeled nucleotide.

Capping the labeled nucleotide may increase the rate of incorporation of the additional nucleotide adjacent to the labeled nucleotide. The additional nucleotide may comprise or be coupled to a detectable moiety by a linker. The method may comprise detecting the detectable moiety coupled to the additional nucleotide, for example following its incorporation. A linker coupled to the additional nucleotide may be cleavable. Cleavage of the linker coupled to the additional nucleotide may also decouple a capping reagent or portion of a capping reagent from the labeled nucleotide.

### Dark Tap Methods

A method consistent with the present disclosure may comprise incorporating a first nucleotide coupled to a linker comprising a detectable moiety into a nucleic acid strand; detecting the detectable moiety; cleaving the linker, thereby generating a scar moiety on the first nucleotide; contacting the nucleic acid strand with a mixture comprising a capping reagent and a second nucleotide, wherein the capping reagent is configured to couple to the scar moiety on the first nucleotide. The first and second nucleotides may be provided as pluralities of nucleotides, which pluralities may comprise single or multiple types of canonical nucleotides (e.g., only deoxyadenosine triphosphate or a combination of deoxyadenosine triphosphates and thymidine triphosphates). The pluralities of nucleotides may comprise about 100% labeled (e.g., coupled to linkers comprising detectable moieties) nucleotides, combinations of labeled and unlabeled nucleotides (e.g., each canonical type of nucleotide present in a plurality of nucleotides comprises 50% labeled and 50% unlabeled nucleotides), or in the case of the second nucleotide, as a plurality of about 100% unlabeled nucleotides. Contacting a nucleic acid with only unlabeled nucleotides may be referred to as a “dark tap” or “dark tap step,” as a nucleotide may lack a detectable moiety to identify its incorporation into the nucleic acid. Contacting a nucleic acid with a labeled nucleotide may be referred to as a “bright tap” or “bright tap step,” as a nucleotide may comprise a detectable moiety which may provide a means for identifying its incorporation into the nucleic acid molecule.

In some cases, the nucleic acid strand is hybridized or partially hybridized to a second nucleic acid. A nucleotide incorporation into the nucleic acid strand may identify a nucleotide of the second nucleic acid strand. Multiple nucleotide incorporations into the nucleic acid strand may identify a sequence or a portion of a sequence of the second nucleic acid strand. A method may comprise a series of “bright taps” and “dark taps.” For example, the method may comprise alternating “bright taps” comprising labeled deoxyadenosine triphosphate and labeled deoxyguanosine triphosphate molecules with “dark taps” comprising unlabeled deoxycytidine triphosphate and unlabeled thymidine triphosphate molecules. Such a method may identify at least a portion of thymidine and cytidine nucleotides in a sequence. In some cases, such as the sequencing of a characterized genome, identification of only one, two, or three canonical nucleotide types may be sufficient to unambiguously identify a nucleic acid sequence. For example, some human genes can often be identified with 20 or fewer base pair identifications.

A method consistent with the present disclosure may comprise incorporating a first nucleotide coupled to a linker comprising a detectable moiety into a nucleic acid strand; detecting the detectable moiety; cleaving the linker, thereby generating a scar moiety on the first nucleotide; contacting the nucleic acid strand with a mixture comprising a capping reagent and a second nucleotide, wherein the capping reagent is configured to couple to the scar moiety on the first nucleotide; wherein the second nucleotide is unlabeled and is of the same canonical base type as the first nucleotide. Such a method may diminish sequencing misphasing. Some labeled nucleotides may incorporate with less than 100% efficiency (e.g., in fewer than 100% of occurrences in which they properly base pair with the template strand at the position directly adjacent to the 3′ end of the growing strand). Furthermore, when a template strand hybridized to the nucleic acid strand comprises a homopolymeric region, the incorporation of the first nucleotide may block subsequent nucleotide incorporations. Follow a “bright tap” with a “dark tap” comprising the same canonical type of nucleotide may complete an extension through a complete homopolymer region.

### Computer Systems

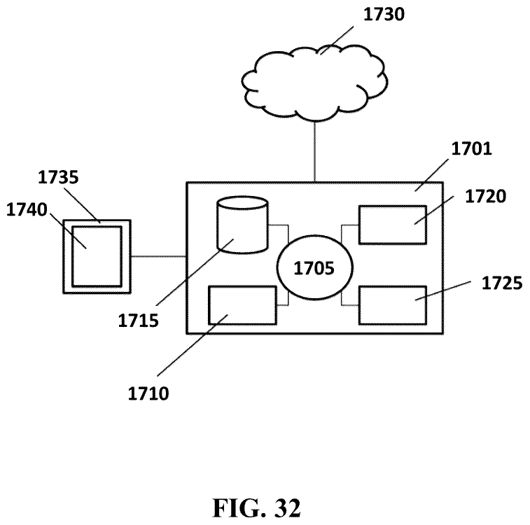

The present disclosure provides computer systems that are programmed to implement methods of the disclosure. FIG. 32 shows a computer system 1701 that is programmed or otherwise configured to perform nucleic acid sequencing. The computer system 1701 can determine sequence reads based at least in part on intensities of detected optical signals. The computer system 1701 can regulate various aspects of the present disclosure, such as, for example, performing nucleic acid sequencing, sequence analysis, and regulating conditions of transient binding and non-transient binding (e.g., incorporation) of nucleotides. The computer system 1701 can be an electronic device of a user or a computer system that is remotely located with respect to the electronic device. The electronic device can be a mobile electronic device.

The computer system 1701 includes a central processing unit (CPU, also “processor” and “computer processor” herein) 1705, which can be a single core or multi core processor, or a plurality of processors for parallel processing. The computer system 1701 also includes memory or memory location 1710 (e.g., random-access memory, read-only memory, flash memory), electronic storage unit 1715 (e.g., hard disk), communication interface 1720 (e.g., network adapter) for communicating with one or more other systems, and peripheral devices 1725, such as cache, other memory, data storage and/or electronic display adapters. The memory 1710, storage unit 1715, interface 1720 and peripheral devices 1725 are in communication with the CPU 1705 through a communication bus (solid lines), such as a motherboard. The storage unit 1715 can be a data storage unit (or data repository) for storing data. The computer system 1701 can be operatively coupled to a computer network (“network”) 1730 with the aid of the communication interface 1720. The network 1730 can be the Internet, an internet and/or extranet, or an intranet and/or extranet that is in communication with the Internet. The network 1730 in some cases is a telecommunication and/or data network. The network 1730 can include one or more computer servers, which can enable distributed computing, such as cloud computing. The network 1730, in some cases with the aid of the computer system 1701, can implement a peer-to-peer network, which may enable devices coupled to the computer system 1701 to behave as a client or a server.

The CPU 1705 can execute a sequence of machine-readable instructions, which can be embodied in a program or software. The instructions may be stored in a memory location, such as the memory 1710. The instructions can be directed to the CPU 1705, which can subsequently program or otherwise configure the CPU 1705 to implement methods of the present disclosure. Examples of operations performed by the CPU 1705 can include fetch, decode, execute, and writeback.

The CPU 1705 can be part of a circuit, such as an integrated circuit. One or more other components of the system 1701 can be included in the circuit. In some cases, the circuit is an application specific integrated circuit (ASIC).

The storage unit 1715 can store files, such as drivers, libraries, and saved programs. The storage unit 1715 can store user data, e.g., user preferences and user programs. The computer system 1701 in some cases can include one or more additional data storage units that are external to the computer system 1701, such as located on a remote server that is in communication with the computer system 1701 through an intranet or the Internet.

The computer system 1701 can communicate with one or more remote computer systems through the network 1730. For instance, the computer system 1701 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1701 via the network 1730.

Methods as described herein can be implemented by way of machine (e.g., computer processor) executable code stored on an electronic storage location of the computer system 1701, such as, for example, on the memory 1710 or electronic storage unit 1715. The machine executable or machine readable code can be provided in the form of software. During use, the code can be executed by the processor 1705. In some cases, the code can be retrieved from the storage unit 1715 and stored on the memory 1710 for ready access by the processor 1705. In some situations, the electronic storage unit 1715 can be precluded, and machine-executable instructions are stored on memory 1710.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 1701, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1701 can include or be in communication with an electronic display 1735 that comprises a user interface (UI) 1740 for providing, for example, results of nucleic acid sequence and optical signal detection (e.g., sequence reads, intensity maps, etc.). Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1705. The algorithm can, for example, implement methods and systems of the present disclosure, such as determine sequence reads based at least in part on intensities of detected optical signals.

### EXAMPLES

### Example 1: A Structure of a Labeling Reagent

Described herein is an example of a semi-rigid, water-soluble linker of a defined molecular weight that can efficiently accomplish a dye-dye or dye-quencher separation. A semi-rigid structure can be achieved through a series of linked, aromatic, or non-aromatic ring systems connected by zero or one linkages with sp³ bonding, and zero or more sp or sp² bonds. Water-solubility can be achieved with the inclusion (e.g., in each subunit) of at least one of the moieties selected from the group: hydroxyl, pyridinium, imidazolium, sulfonate, amino, thiol, carboxyl, and quaternary ammonium. A linker can be a multifunctional reagent (e.g., heterobifunctional, heterotrifunctional, homobifunctional, or homotrifunctional) that allows attachment of a dye (e.g., fluorescent dye) at a first site and a biological ligand (e.g., a nucleotide) at a second site, and which optionally contains additional sites configured to couple to additional species. An example of a general formula for such a linker is shown below:

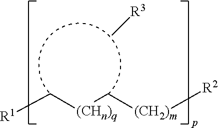

in which p is a number of repeating units selected from 1-100; each R³ is a water-soluble moiety independently selected from, for example, pyridinium and sulfonate; R¹ and R² are attachment groups such as amino and carboxy moieties; each n is independently 1 or 2; each m is independently selected from 1 and 2; and each q is independently selected from 4-8. In the structure above, m represents the number of sp³ carbons linking ring moieties to one another. A ring moiety may be an aliphatic or an aromatic ring.

Multiple such subunits may be connected to one another. For example, a linker may be represented by the below formula:

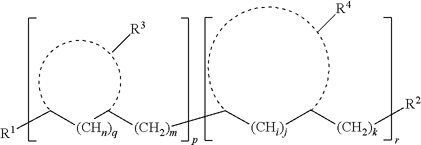

in which p and r are each a number of repeating units independently selected from 1-100; each R³ and R⁴ is a water-soluble moiety independently selected from, for example, pyridinium and sulfonate; R¹ and R² are attachment groups such as amino and carboxy moieties; each of n and i is independently selected from 1 or 2; each of m and k is independently selected from 1 and 2; and each of q and j is independently selected from 4-8. In the structure above, m and k each represent the number of sp³ carbons linking ring moieties to one another. A ring moiety may be an aliphatic or an aromatic ring. In some cases, ring moieties in the left portion of the structure are aliphatic and ring moieties in the right portion of the structure are aromatic, or vice versa.

A linker may attach to a substrate and a detectable moiety. For example, R¹ may attach to a nucleotide and R² may attach to a fluorescent dye. Furthermore, R¹ may comprise a cleavable group, such as a disulfide. For example, an R¹ moiety may comprise

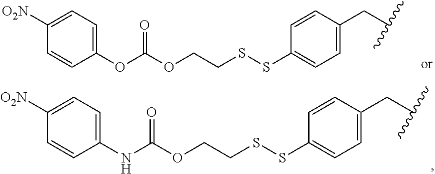

which may couple to a propargyl alcohol or propargyl amine group on a substrate.

Note that the above structures do not capture all embodiments of the disclosure. For example, the linker does not have to be a polymer of “P-repeating” units. Similarly, the water-soluble functional group can be a constituent component of the ring rather than attached to the ring.

### Example 2: Synthesis of a Capped Nucleotide Labeling Reagent

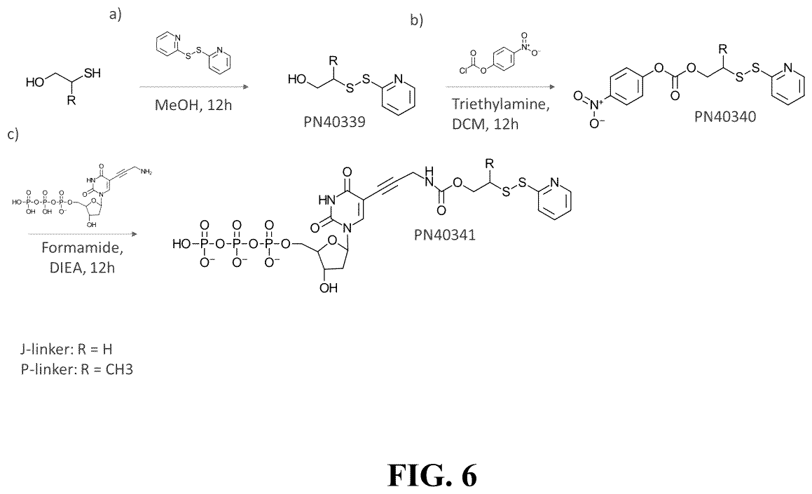

This example illustrates a synthesis method for a labeled nucleotide, as shown in FIG. 6. While this method provides a capped nucleotide, the pyridinyl moiety on the disulfide can be swapped for a wide range of moieties, for example an oligopeptide or polypeptide linker coupled to a detectable moiety. The modularity of this method can thus facilitate large library generation through variations of the disulfide reagent in step a).

The first step of this method (FIG. 6 step (a)) comprises a thiol-disulfide exchange to generate the disulfide of the labeled nucleotide product. The disulfide may serve as a cleavable group of the mature linker. 2,2′-Dipyridyldisulfide (0.389 g, 2.0 mmol) was dissolved in 2 mL methanol, and then combined with 2-Mercapto-1-propanol (0.108 g, 1.0 mmol). The resulting reaction mixture was left at room temperature overnight. The reaction mixture was then spotted onto a prep-TLC plate and eluted in a 2:1 hexanes:ethyl acetate solution. The silica gel with the product spot was scraped off and washed with ethyl acetate, which was evaporated to collect product (2-(2-Pyridyldithio)-1-propanol). Step (a) can accommodate a wide range of thiol and disulfide substituents.

The second step (FIG. 6 step (b)) comprises reactive carbonate formation on the disulfide linker, thereby enabling coupling to a range of moieties on a substrate, including amines (e.g., propargyl amines), and thiols. In the synthesis shown, the product from step (a) (2-(2-Pyridyldithio)-1-propanol, 0.062 g, 0.31 mmols) was dissolved in 1 mL dichloromethane and combined with triethylamine (0.047 mL, 0.34 mmols). p-(N-Hydroxyhydroxyamino)benzenyl chloroformate (0.092 g, 0.46 mmol) was dissolved in 1 mL dichloromethane and added to the reaction mixture, which was stirred overnight under nitrogen. The reaction mixture was then spotted onto a prep-TLC plate and eluted in a 2:1 hexanes:ethyl acetate solution. The silica gel with the product spot was scraped off and washed with ethyl acetate, which was evaporated to collect product (PN40340).

The third step (FIG. 6 step (c)) comprises nucleophilic substitution by a nucleophile coupled to the substrate (in this case a nucleotide propargylamine) at the carbonate of the product of step (b). In the synthesis shown, A 0.1M solution of PN40340 was prepared in formamide, 0.15 mL of which was combined with 0.05 mL of a 0.1M solution of dUTP-AP in formamide with 17 uL of diisopropylethylamine. The reaction mixture was left overnight, and then purified by HPLC to yield the labeled nucleotide product. The substrate and the reactive moiety may be varied in this step. For example, the substrate may be any nucleotide, nucleoside, amino acid, lipid, or metabolite. The reactive moiety on the substrate may be an alcohol (thereby yielding a carbonate linker) or a thiol (thereby yielding a thiocarbonate) along with a range of other nucleophiles. Thus, the synthesis presented in FIG. 6 is highly modular and able to accommodate a wide range of substrates, linkers, and detectable moieties. Furthermore, the reagents for the steps in a-c may be varied so as to generate linkers that immolate upon disulfide cleavage or linkers that do not react further following disulfide cleavage. The rate of the immolation reaction may also be tailored based on choice of reagents. For example, a multiply alkylated thiol in step a may generate a linker with a relatively fast immolation rate, while the use of less substituted thiols (e.g., 2-mercaptoethan-1-ol) may generate a linker with a relatively slow immolation rate.

### Example 3: Labeled Nucleotide Linker Cleavage and Immolation

This example covers cleavage and immolation of linkers coupled to nucleotide substrates. A linker may comprise a cleavable group that generates a scar moiety on its substrate upon cleavage. The linker may be configured such that the scar moiety generated from cleavage undergoes spontaneous immolation. The immolated scar may comprise favorable features relative to the non-immolated scar. For example, an immolated scar may comprise less steric bulk, a higher pH, and a lower Lewis basicity than the post-cleavage scar from which it forms, which can increase its performance in an enzyme-based assay, such as nucleic acid sequencing-by-synthesis. In order to be suitable for an assay, a linker may also need to have fast cleavage and immolation kinetics. The present example provides two linkers with similar structures but drastically different immolation kinetics, demonstrating that linkers can be optimized for cleavage and immolation.

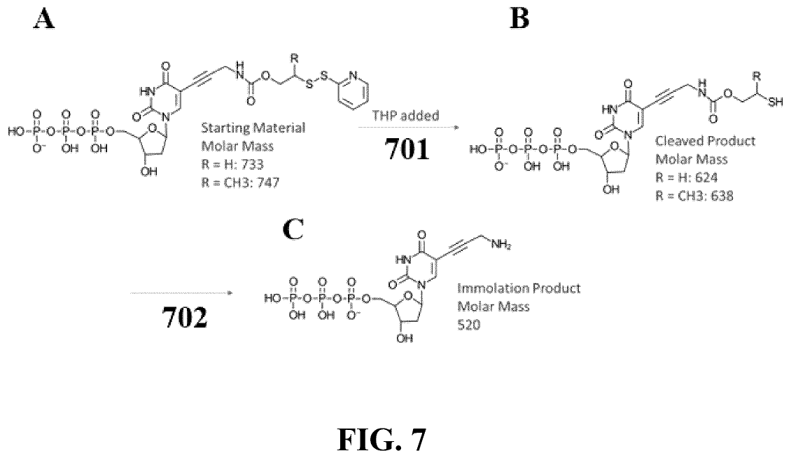

FIG. 7 provides a chemical scheme for linker cleavage and immolation in two labeled uridine triphosphate substrates. Panel A provides the structures of the two labeled substrates, one with a methyl group at the variable position ‘R’ (“P-linker”) and one with hydrogen at the variable position ‘R’ (“J-linker”). A cleavage step 701 can be initiated by the reducing agent THP, reducing and cleaving the linker disulfides to yield the cleavage products shown in panel B. Alternate schemes may utilize different reducing agents, such as methyl viologen or hydrazine. Following cleavage, the products of panel B undergo immolation reactions 702 yielding propargyl amine-functionalized uridine triphosphate.

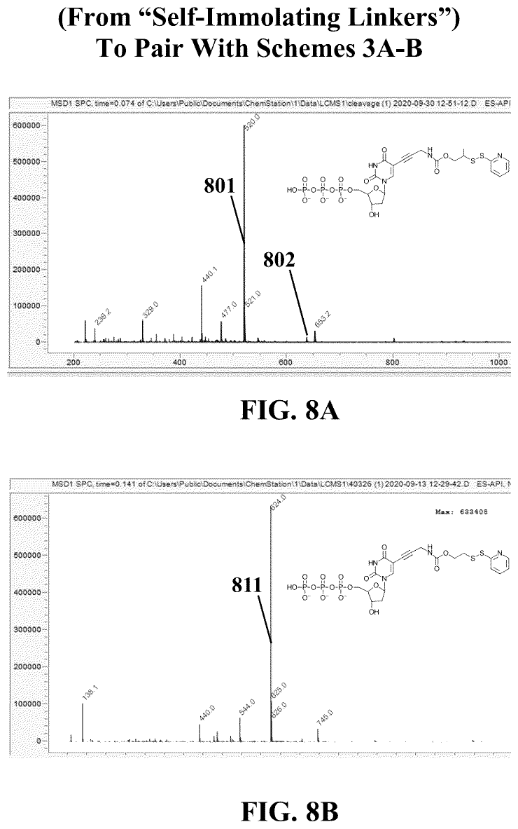

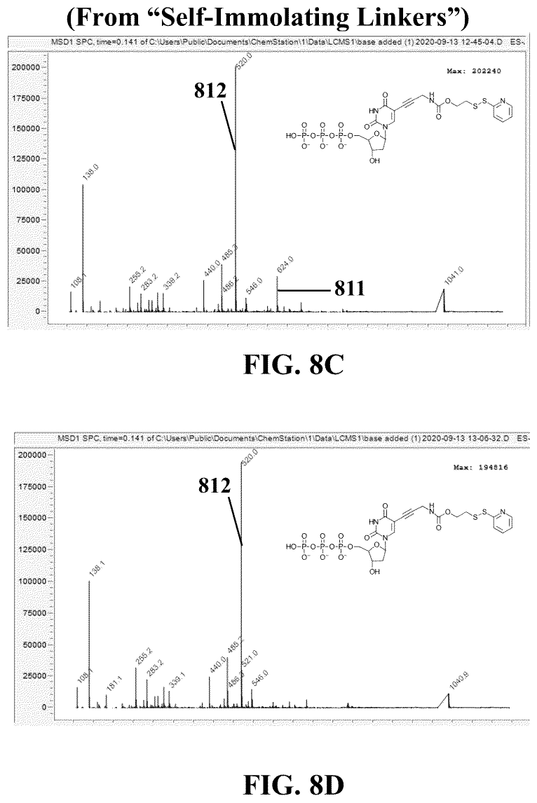

FIG. 8 provides mass spectra of the cleavage and immolation products of the P-linker and J-linker reactions shown in FIG. 7. FIGS. 8A and 8B, show the products of the P-linker and J-linkers immediately following THP addition (step 701 in FIG. 7). In FIG. 8A, which shows the products of the P-linker cleavage and immolation reactions, the largest peak 801 (at an m/z of 520) corresponds to the immolation product uridine triphosphate (shown in FIG. 7 panel C), while the post-cleavage thiol product (shown in FIG. 7 panel B), with a mass spectrometric signal 802 at an m/z of 636, is nearly entirely absent. Conversely, for the J-linker reaction products shown in FIG. 8B, the largest peak 811 (at an m/z of 624) corresponds to the post-cleavage thiol product, while the immolation product is undiscernible. For both the P-linker and J-linker, the starting, pre-cleavage reagents are nearly entirely absent, indicating that disulfide cleavage is nearly spontaneous in the presence of THP

FIG. 8C provides a mass spectrum of the J-linker reaction products 10 minutes after THP addition (to initiate cleavage). Relative to the immediate-post cleavage spectrum of FIG. 8B, the peak corresponding to the post-cleavage, pre-immolation product 811 (at an m/z of 624) has nearly entirely disappeared, while a peak corresponding to the post-immolation product 812 (at an m/z of 520) is the largest peak, showing that the final, post-immolation product is the predominant species present. The ratio of peaks 811 and 812 indicate a first order rate constant of roughly 0.2 min⁻¹ (or a half-life of roughly 3.5 minutes).

FIG. 8D shows the mass spectrum of the J-linker 30 minutes post THP addition. In this spectrum, the peak corresponding to the post-cleavage, pre-immolation product is no longer discernible, while the peak corresponding to the product 812 is the largest peak present, thus indicating that the cleavage and immolation reactions both proceed to completion after this time.

### Example 4: Fluorophore Quenching in Multi-Label Nucleic Acid Homopolymers

This example covers fluorescence quenching in multiply labeled nucleic acid molecules. Quenching poses a major challenge to many forms of fluorescence analysis. In nucleic acid sequencing, multiple fluorophores coupled to a single nucleic acid molecule may participate in self-quenching, thereby lowering the total fluorescence emission from the nucleic acid molecule and adding to the challenge of fluorophore quantitation.

Two labeled uridine nucleotides were tested for homopolymer synthesis performance and self-quenching activity. Both labeled uridine nucleotides contained the hyp10 linker, which includes the sequence Gly-Hyp-Hyp-Hyp-Hyp-Hyp-Hyp-Hyp-Hyp-Hyp-Hyp from the N-terminal end. The first, U*-YHH (shown in FIG. 9A) contained two consecutive hyp10 linkers, while the second U*-YH (shown in FIG. 9B) contained a relatively shorter linker with only one hyp10. The labeled nucleotides were provided for primer extension across 1 to 6 consecutive adenosine regions of a template strand. FIGS. 9C-9D provide imaging results of electrophoretic separation of nucleic acid molecules comprising 1 to 6 consecutive uridines. Columns A1-A6 correspond to template strands containing 1 to 6 consecutive adenosine nucleotides, respectively (e.g., the A3 template strand contains 3 consecutive adenosines directly 5′ of the primer hybridized region). The 1-, 2-, 3-, 4-, 5-, and 6-mer uridine homopolymer products are indicated as 901, 902, 903, 904, 905, and 906 respectively in FIG. 9C and as 911, 912, 913, 914, 915, and 916 in FIG. 9D. As can be seen from these images, partial extension products (e.g., a 4-mer uridine extension product against the A5 template) were also observed. The brightness of each spot corresponds both to the amount of each product formed, and to the degree to which quenching was minimized by the labeled nucleotide linkers.

FIG. 9E provides relative fluorescence intensity (y-axis) vs number of labeled uridine nucleotides incorporated into the primer (x-axis). Both the U*-YHH and U*-YH nucleotides provide sub-linear fluorescence responses. For U*-YH, the fluorescence intensity maximizes for a 3-mer structure, with 2- through 6-mer homopolymers exhibiting relatively similar levels of fluorescence. U*-YHH provides higher levels of fluorescence and exhibits fluorescence intensity increases with each added labeled nucleotide. These data demonstrate the high level of challenge associated with designing low self-quenching efficiency labeled nucleotides, and that labeled nucleotides of the present disclosure can overcome this challenge by sufficiently diminishing quenching so as to allow various length homopolymer regions to be distinguished.

### Example 5: Polymerase Inhibition by Scarred Nucleotides

This example covers potential inhibitory effects imparted by labeled nucleotide scars. A nucleotide may be coupled to a detectable moiety (e.g., a fluorescent dye) by a linker. Following incorporation of the nucleotide into a growing nucleic acid strand, the linker may be cleaved, which can yield a scar (e.g., a portion of the linker) coupled to the incorporated nucleotide. The scar may inhibit subsequent nucleotide additions to the growing nucleic acid strand and may increase the frequency of mispair additions. A strategy for diminishing scar adverse effects is capping scars in a growing nucleic acid strand.

In this example, a set of labeled nucleotide reagents were contacted to a primer hybridized to a template strand comprising the 3′ sequence TCAGTGGTTG under conditions suitable for extension. Fluorescently labeled adenosine, cytidine, and guanosine nucleotides were of the structure indicated in FIG. 10A, while labeled uridine nucleotides were of the structure provided in FIG. 10B. Different canonical nucleotide types were provided in separate, sequential flows, starting with uridine (U), followed by adenosine (A), cytidine (C), and finally guanosine (G), before repeating the cycle beginning again with U. Flows contained anywhere from 10% to 100% labeled nucleotides, with the remaining nucleotides provided in their natural, unlabeled form. In between each flow, the fluorescent labels were detected, cleaved, and optionally capped. Each labeled nucleotide flow was then followed by a “dark tap” flow consisting of 100% unlabeled nucleotides in order to maintain phasing.

FIG. 10C summarizes the sequencing results of an assay utilizing flows with 100% labeled U, 10% labeled A, 20% labeled C, 10% labeled G, and no capping reagents. From top to bottom, the plots in FIGS. 10C-10E provide measured fluorescence intensities following U, A, C, and G flows, respectively, with individual cycles progressing from left to right. As can be seen from the plots, the initial U incorporation (top left feature in the top plot) and subsequent linker cleavage inhibited the next three incorporations of C, A, and then G. While A, C, and G incorporations minimally inhibited subsequent labeled reagent incorporations, this may be due to the relatively low fractions of labeled nucleotides in the U, C, and G flows.

FIG. 10D summarizes the sequencing results of an assay utilizing flows with 100% labeled U, 10% labeled A, 100% labeled C, 10% labeled G, and no capping reagents. The first nucleotide incorporation with 100% labeled U inhibited the second nucleotide incorporation with 100% labeled C. In spite of the 100% C labeling, the signal from the 2^{nd} nucleotide incorporation (indicated with the dashed circle) yielded a relatively low fluorescence signal. The signals corresponding to the third and fourth nucleotide incorporations (top and bottom solidly-circled features) were also suppressed, indicating that scarred nucleotides inhibit the next three incorporation steps.

FIG. 10E summarizes the sequencing results of an assay utilizing flows with 100% labeled U, 10% labeled A, 20% labeled C, 10% labeled G, and the capping reagent DPDS. provided in only the U “dark tap” nucleotide flows. As can be seen from the circled signal corresponding to incorporation of an A, the capping reagent provided with the U-flow cycles increased the incorporation efficiency for the next flow (in this case A). However, flows not directly following U did not exhibit improved incorporation efficiencies. This was likely due to capping reagent cleavage from the nucleotide scars, as the linker cleavage reagent was likely also active in removing caps from scar moieties. Since each labeled nucleotide flow was followed with a cleavage reagent, scars likely only remained capped through labeled A incorporation, and thus would not have been present during C and G incorporations. This limitation could potentially be removed by providing a capping reagent in each “dark tap” step, rather than only in the “dark tap” steps following labeled uridine incorporation.

### Example 6: Capping Reagent Effects on Extension Efficiency

This example covers the effects of scar capping on extension efficiency. Cleaving a linker coupling a nucleic acid to a dye may improve the efficiency of subsequent nucleotide additions into the nucleic acid. In some cases, the efficiency can be further improved by capping a scar left over from linker cleavage.

Extension and sequencing performance were tested for a set of labeled nucleotides and capping reagents. The labeled nucleotides used in this example consisted of the dye-linkers as shown in FIG. 9A on each of the four nucleotides. A set of sequencing assays were performed by extending a primer against a template nucleic acid sequence in the presence of alternating flows of single fluorescently labeled nucleotides provided as cycles of U, A, C, and then G. In between each flow, the fluorescent labels were detected, cleaved, and optionally capped with one of ethyl propiolate (EP), iodoacetamide (IAc), methyl methanethiosulfonate (MMTS), or dipyridyl disulfide (DPDS). Each labeled nucleotide flow was then followed by a “dark tap” flow consisting of 100% unlabeled nucleotides to maintain phasing.

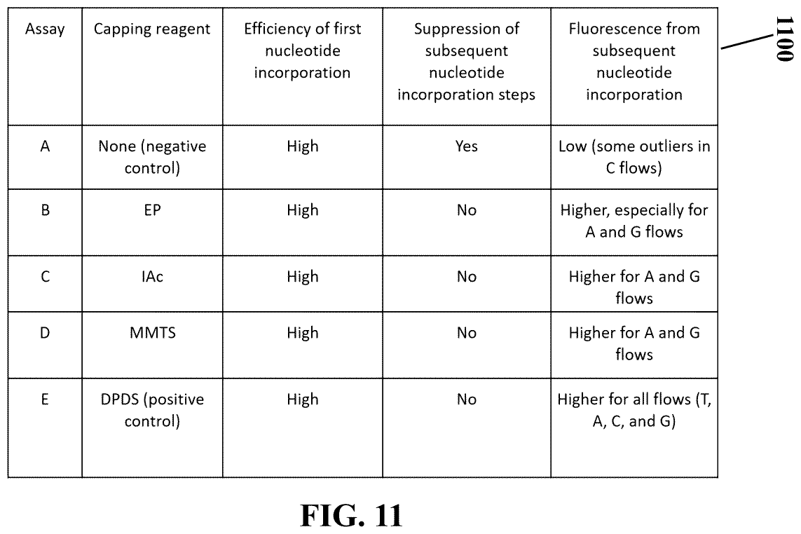

FIG. 11 summarizes the results of the sequencing assays in table 1100. Table 1100 provides the results of uridine, adenosine, cytidine, and guanosine incorporation steps performed without any capping agents or in the presence of different capping agents. Assay A illustrates results from sequencing assays performed without capping reagents. Following a high-efficiency first nucleotide incorporation step with labeled uridine, subsequent nucleotide incorporation steps were suppressed, including subsequent uridine additions. Comparatively, assays B-E, which included EP, IAc, MMTS, and DPDS capping reagents, respectively, exhibited high intensities for subsequent incorporation steps (e.g., lack of suppression). In all four assays, the relative intensities from the second nucleotide incorporation, a cytidine, were comparable to the initial, uridine incorporation (data not shown). Fluorescence intensities from subsequent nucleotide incorporations were also higher in the assays with capping reagents (assays B-E) than without capping reagents (assay A), with the DPDS assay (assay E) exhibiting the highest intensities. These results show that capping reagents can minimize the inhibitory effects of scar moieties and enable high rates of labeled nucleotide incorporations, with DPDS being the most effective capping reagent.

### Example 7: Homopolymer Extension with Scarred and Capped Reagents

This example covers homopolymer extension during nucleic acid synthesis with labeled reagents. Limiting the number of labels coupled to a nucleic acid can minimize quenching, thereby enabling label number quantitation (e.g., determining the length of a homopolymeric region by identifying the number of adjacent nucleotides disposed within a growing nucleic acid strand). One such strategy for limiting the number of labels coupled to a nucleic acid is to introduce label cleavage steps. However, the scars generated through cleavage can cause further problems, such as the inhibition of subsequent nucleotide incorporations. As disclosed herein, capping a scar can mitigate adverse its adverse effects. This example compares homopolymer extension with uncleaved, scarred, and capped-cleaved nucleotides.

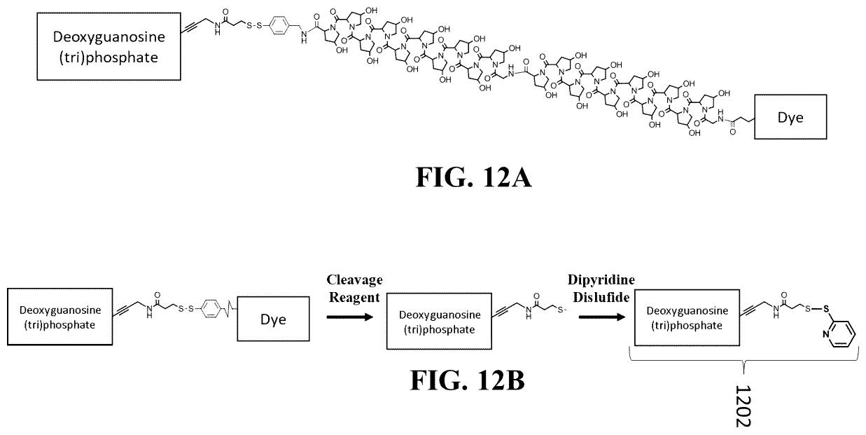

Three sequencing assays were performed by extending a primer over 1- to 6-mer cytidine regions of template nucleic acids. The first assay utilized pure labeled guanosine triphosphates (GTP) with fluorescent dyes on 20-mer hydroxyproline linkers, as shown in FIG. 12A. The second assay utilized a 1:1 mixture of labeled and unlabeled GTP. The third assay utilized a 1:1 mixture of labeled GTP and scar-capped GTP, see structure 1202 in FIG. 12B, generated by disulfide cleavage and dipyridine disulfide capping of the labeled GTP, as illustrated in FIG. 12B. The fluorescence of each product was measured following extension over the 1- to 6-mer cytidine template nucleic acid regions.

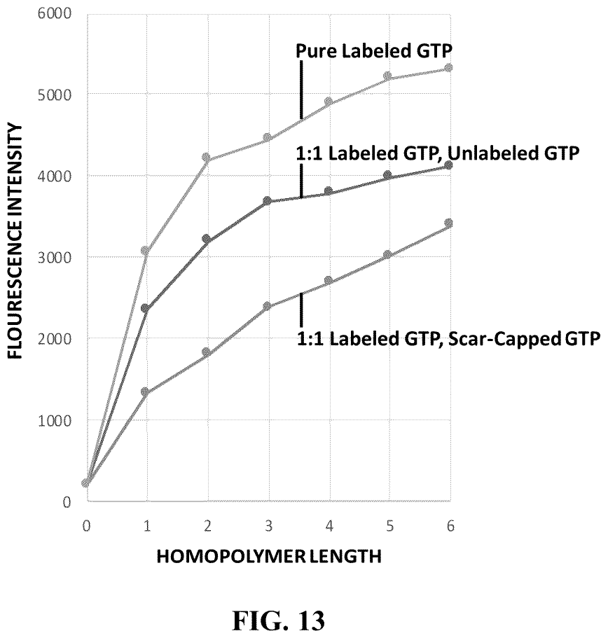

The results of the assays are summarized in FIG. 13, which provides fluorescence readout (y-axis) vs. template nucleic acid oligocytidine region length (i.e., 0, 1, 2, 3, 4, 5, or 6 consecutive cytidines). The highest fluorescence intensities were observed for the products of the pure labeled GTP assay, even for the longer oligoguanosine products (e.g., the 4-, 5-, and 6-mers), indicating that the hydroxyproline linkers created sufficient spacing between dyes to minimize quenching. The assay utilizing 1:1 labeled and unlabeled GTP generated products with the second highest fluorescence intensities. While the intensity vs product-length trend mirrored that of the pure labeled GTP assays, the fluorescence intensities were greater than half of those from the pure labeled GTP assays, indicating that the labeled and unlabeled GTP mixture exhibited lower quenching. The assays utilizing the labeled GTP, scar-capped GTP mixture exhibited the lowest fluorescence intensities. However, these assays also exhibited the most linear trend between oligoguanosine product length and fluorescence intensity, as well as the greatest fluorescence intensity differences between the 3-, 4-, 5-, and 6-mer oligoguanosine products. These results demonstrate the viability of all three methods for sequencing nucleotide homopolymers.

### Example 8: Cleavable Linker Structure and Scar Immolation Kinetics

This example covers cleavable linker immolation kinetics and sequencing assay performance. Linker cleavage can generate scar moieties that inhibit polymerase activity. The present disclosures provide linkers that immolate upon cleavage, yielding smaller and less inhibitory scars. In this example, labeled uridine nucleotides coupled to fluorescent dyes by cleavable, immolating linkers were incorporated into a homopolymeric region of a nucleic acid strand. The rates of subsequent nucleotide additions were then measured in the presence and absence of cleavage reagents.

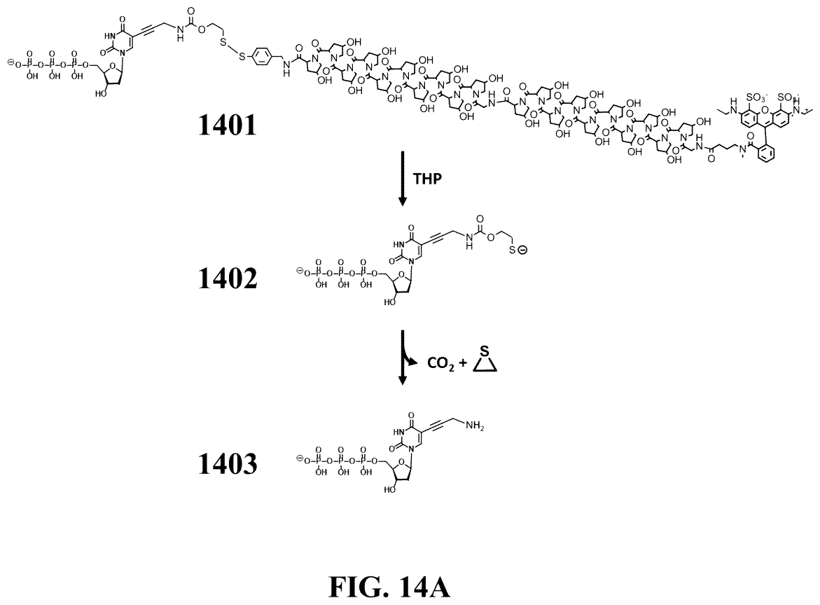

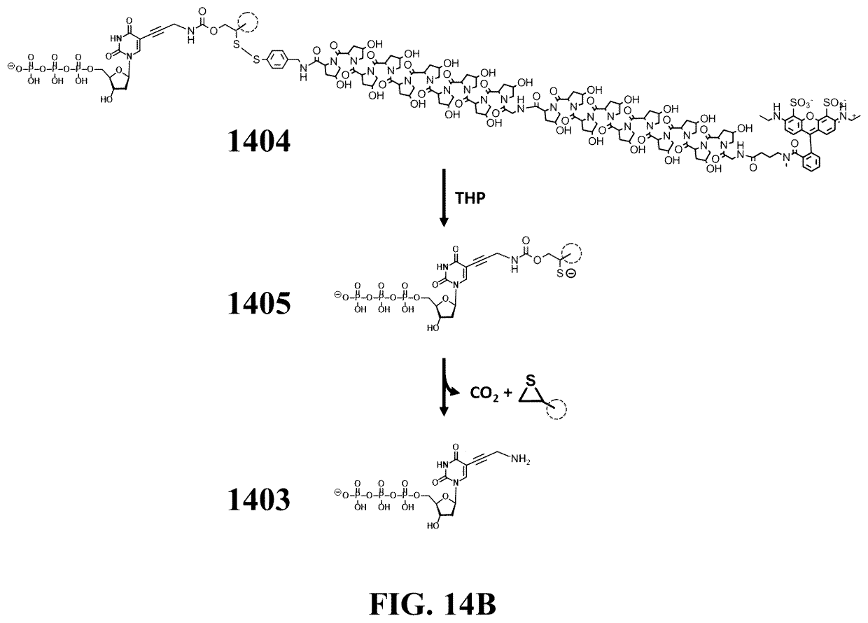

The structures of the two labeled nucleotides (provided in FIGS. 14A and 14B as 1401 and 1404) differ by a single methyl substituent present in 1404 (circled) and absent in 1401. The disulfides of both labeled nucleotides can be cleaved by reducing agents, such as THP, yielding the thiolates shown as 1402 in FIG. 14A and 1405 in FIG. 14B. The thiolate cleavage products can then undergo spontaneous immolation reactions, liberating CO₂ and thiirane in the case of 1402 immolation, and liberating CO₂ and methyl thiirane in the case of 1405 immolation. Both immolation reactions yield the propargyl amine product 1403.

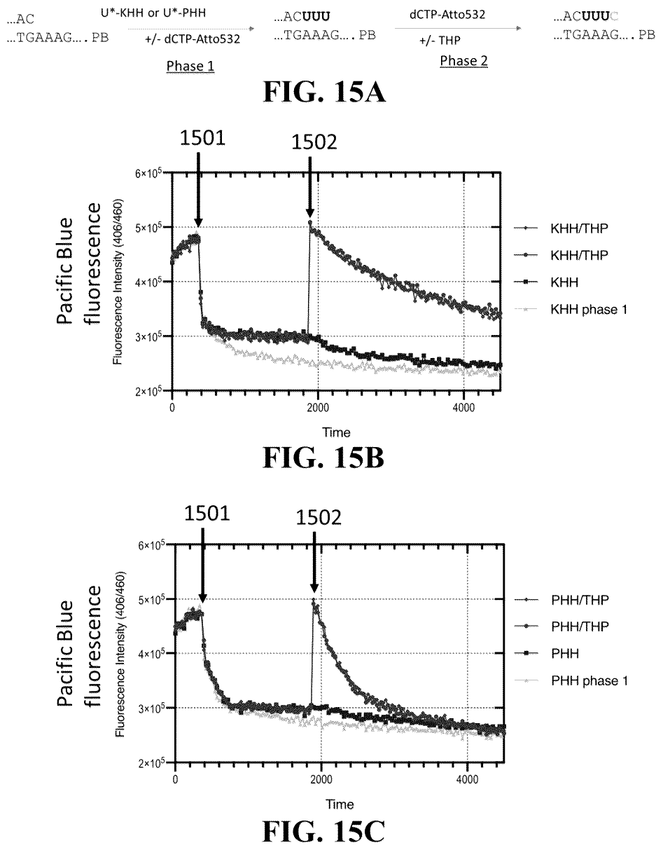

Both labeled nucleotides (1401 and 1404 from FIGS. 14A and 14B) were provided in a sequencing assay measuring extension over a template region comprising the sequence AAAG. The assay scheme is provided in FIG. 15A. After hybridizing a primer to the template strand, extension was performed in the presence of the labeled uridine nucleotides, leading to three consecutive uridine incorporations. Extension was then performed in the presence of dye-labeled dCTP. In some cases, this step included the disulfide cleaving reagent THP.

The results of the assays are shown in FIGS. 15B and 15C, with FIG. 15B providing results obtained with the labeled nucleotide 1401 in FIG. 14A, and FIG. 15C providing results obtained with the labeled nucleotide 1404 in FIG. 14B. Labeled uridine addition 1501 is quickly followed by a change in fluorescence. In the absence of THP, the resulting fluorescence level changes minimally in the presence of dCTP. In the presence of THP, labeled dCTP addition 1502 results in a fluorescence spike, followed by an apparent first order decay, representing uridine linker immolation (subsequent to rapid disulfide cleavage). The results indicate that the labeled nucleotide 1404 in FIG. 14B undergoes a faster immolation reaction (with a half-life on the order of 5 minutes) than the labeled nucleotide 1401 in FIG. 14A (which exhibits a half-life on the order of 20 minutes).

### Example 9: Cleavable Linker Capping and Immolation

This example covers the effects of scar capping and immolation on polymerase activity. Cleaving a linker coupling a nucleic acid to a dye may diminish fluorescence quenching in an interrogated nucleic acid, which can thereby increase the accuracy of subsequent fluorescent nucleotide detections. However, residual scars from linker cleavage can adversely affect polymerase activity, for example by increasing mispair frequency or decreasing turnover rate. These effects can be diminished by capping or immolating a residual scar to modify one or more of its properties (e.g., pKa).

In this example, a set of sequencing assays were performed by extending a primer against the same template nucleic acid starting with AGTCTTTGGGTT in the presence of alternating flows of single fluorescently labeled nucleotides provided as cycles of U, A, C, and then G. In between each flow, the fluorescent labels were detected, cleaved, and optionally capped with dipyridyl disulfide (DPDS). Each labeled nucleotide flow was then followed by a “dark tap” flow consisting of 100% unlabeled nucleotides to maintain phasing. Furthermore, some assays utilized nucleotides with immolating linkers.

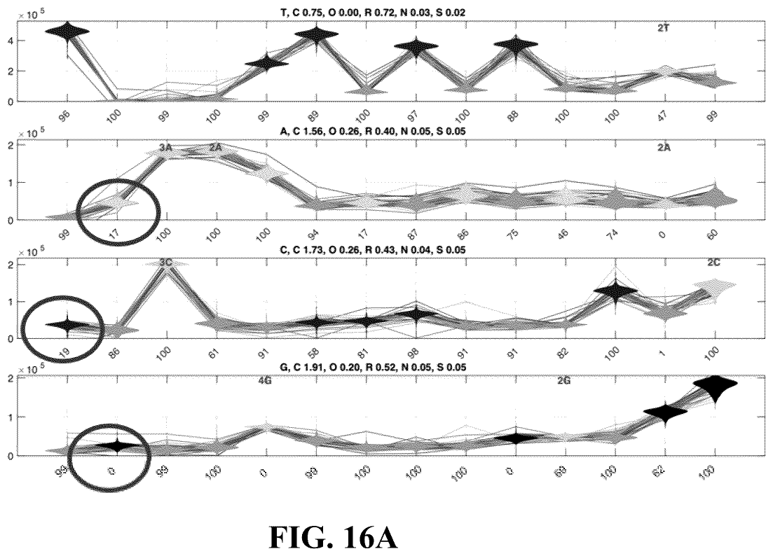

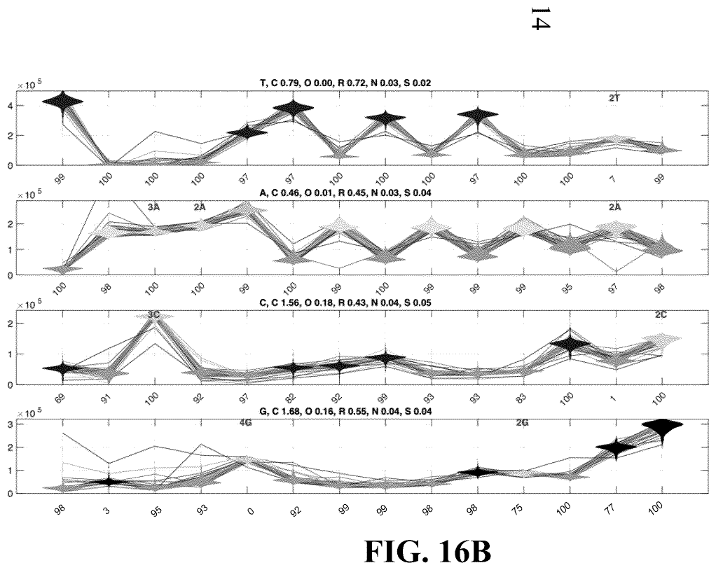

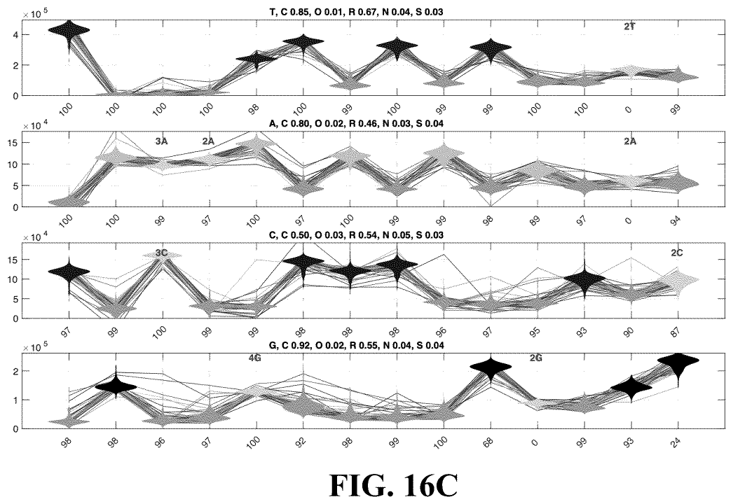

The results of the assays are provided in FIGS. 16A-16C. In each figure, the y-axes provide fluorescence intensity following labeled nucleotide incorporation, and the four plots (from top to bottom) separately provide the results of uridine, adenosine, cytidine, and guanosine incorporation steps, with individual cycles progressing from left to right. In particular, a closer look is taken at the first 4 flow-cycles of U-A-C-G, where nucleotides are expected to be incorporated in the order of T-C (cycle 1; U, A, C, G flows being a complete single cycle), A-G (cycle 2), AAA-CCC (cycle 3), AA (cycle 4). Here nucleotides incorporated in each U-A-C-G flow-cycle are separated with square brackets.

FIG. 16A summarizes the sequencing results of an assay utilizing flows with 100% labeled U, 10% labeled A, 20% labeled C, 10% labeled G, no capping reagent, and non-immolating linkers. In this assay, labeled nucleotide incorporation exhibited noticeable inhibition toward subsequent nucleotide incorporations. For example, following the initial uridine incorporation (the feature at the top left of the top plot), the next three nucleotide incorporations (circled) provided low fluorescence intensities. Intensities of labeled nucleotides further downstream, however, seemed to be little affected. The combined results indicated that the residual linker scar on the incorporated uridine inhibited labeled nucleotide incorporation at three subsequent positions, but incorporation of unlabeled nucleotides in these positions seemed unaffected.

FIG. 16B provides sequencing results for an assay utilizing flows with 100% labeled U, 10% labeled A, 20% labeled C, 10% labeled G. In this assay, the capping reagent DPDS was provided in the U “dark tap” nucleotide flows. Given that the DPDS capping reagent is removed from thiols by disulfide cleaving reagents (including the THP utilized in this assay), scars were capped during A incorporations, and were uncapped for U, C, and G incorporations. The effects of the capping can be seen in the 2^{nd} plot from the top (corresponding to A-incorporations) in FIG. 16B. Compared to FIG. 16A, and further compared to C (3^{rd} plot from the top) and G (4^{th} plot from the top) incorporations, labeled A incorporation was greatly enhanced throughout the entire flow cycle.

FIG. 16C provides sequencing results for an assay utilizing flows with 100% labeled U, 10% labeled A, 20% labeled C, 10% labeled G. In this assay, the labeled U nucleotides contained a spontaneously immolating linker that converted thiol scars to smaller propargyl amine scars upon cleavage. The labeled A, C, and G, nucleotides contained non-immolating linkers. Compared to FIGS. 16A and 16B, fluorescence intensities corresponding to labeled A, C, and G incorporations are enhanced, indicating that immolation of the labeled U linker improves subsequent nucleotide incorporations. Unlike the capping reagent provided in the assay summarized in FIG. 16B, immolation is spontaneous, and does not require repeated reagent additions, as is the case with the capping reagent additions in the assay for FIG. 16B.

### Example 10: Cleavable Linker Capping and Immolation

This example covers nucleotide incorporation efficiency with capped and immolated scarred nucleotides. Removing fluorophores from detected positions during nucleic acid synthesis can increase the accuracy of subsequent labeled nucleotide incorporations. However, fluorophore removal can also result in the formation of scar moieties on incorporated nucleotides, which can inhibit polymerase activity, and diminish the rates of subsequent labeled nucleotide incorporations. Two strategies for diminishing adverse scar moiety effects are designing linkers to immolate upon cleavage and capping scar moieties following linker cleavage.

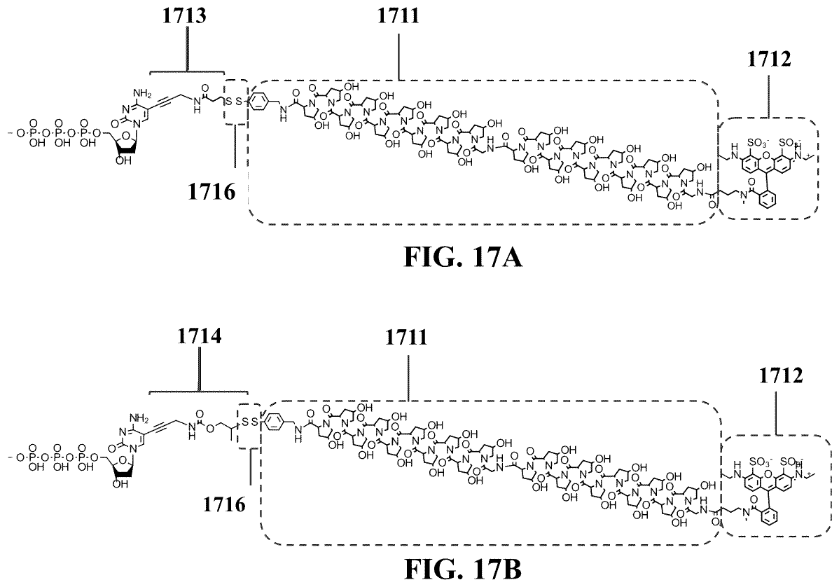

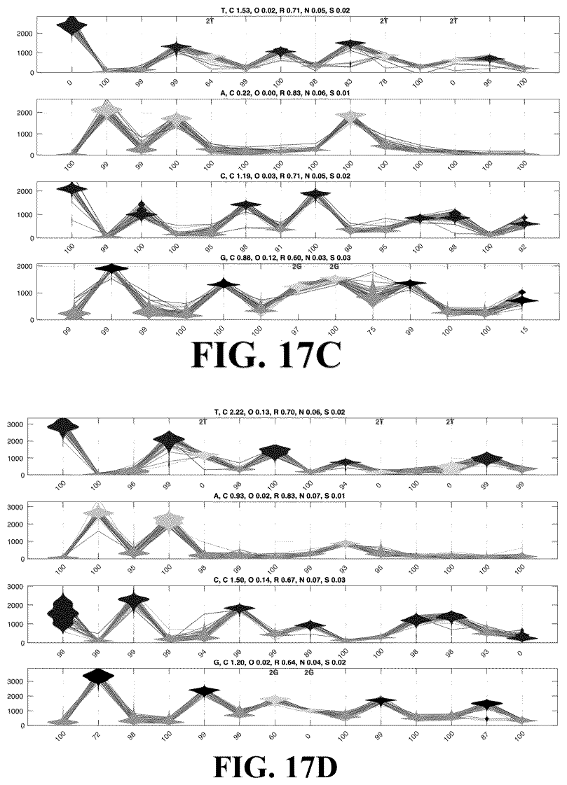

In this example, two types of labeled nucleotides were utilized for nucleic acid sequencing and synthesis, with the structures shown in FIGS. 17A and 17B. Both nucleotides (cytidines shown in these examples) contained cleavable disulfide moieties 1716 configured to release 20-mer hydroxyproline moieties 1711 coupled to dyes 1712. For the labeled nucleotide of FIG. 17A (“Y-type”), the disulfide is coupled to the nucleotide by a propargyl amide 1713 linker that does not immolate under the conditions used in this example The labeled nucleotide shown FIG. 17B (“P-type”) instead contains a propargyl carbamate group 1714 coupling the disulfide to the nucleotide, which can spontaneously immolate upon disulfide cleavage.

FIGS. 17C and 17D provide fluorescence results from nucleic acid sequencing assays utilizing the Y-type and P-type labeled nucleotides, respectively. Both assays utilized labeled nucleotide flows with single canonical nucleotide types, in cycles of T, A, C, and then G (corresponding to the top, 2^{nd} from top, 3^{rd} from top, and bottom plots on each figure). Each labeled nucleotide flow was followed by linker cleavage, and then an unlabeled nucleotide flow of the same canonical nucleotide type to ensure proper phasing. For the Y-type nucleotide assay, the unlabeled nucleotide flows included the thiolate capping reagent DPDS.

FIG. 17C provides the results of the Y-type nucleotide assay, utilizing a non-immolating linker and the capping reagent DPDS. In this assay, labeled nucleotide incorporations do not discernibly inhibit subsequent nucleotide additions. Following the initial labeled thymidine incorporation (top left feature in the top plot), subsequent incorporations display high fluorescence intensities with narrow variations. These results indicate that capped nucleotide scars enable high incorporation rates for new labeled nucleotides.

FIG. 17D provides the results of the P-type nucleotide assay, utilizing an immolating linker and no capping reagents. As in the Y-type nucleotide assay, the results of FIG. 17D reflect high labeled nucleotide incorporation rates over homopolymeric regions, indicating that the P-type linker can minimally inhibit subsequent labeled nucleotide incorporations.

### Example 11: Synthesis of dGTP-AP-SS-hyp10-Atto633

Described herein is a method for constructing the labeled nucleotide dGTP-AP-SS-hyp10-Atto633. FIG. 19 illustrates an example method for the synthesis of a fluorescently labeled dGTP reagent, with the full structures of the dye and linker. The method involves formation of a covalent linkage between Gly-Hyp10 and the fluorophore Atto633 (process (a)), esterification to couple Atto633-Gly-Hyp10 with pentafluorophenol (process (b)), substitution with the linker molecule epSS (process(c)), esterification to form Atto633-Gly-Hyp10-epSS-PFP (process (d)), and substitution with dGTP to provide the fluorescently labeled nucleotide (process (e)). Details of the synthesis are provided below.

Preparation of Atto633-Gly-Hyp10. (FIG. 19 process (a)) A stock solution of Gly-Hyp10 (also referred to herein as “hyp10”) in bicarbonate is prepared by dissolving 25 milligrams (mg) of the 11 amino acid peptides in 500 microliters (μL) of 0.2 molar (M) sodium bicarbonate in a 1.5 milliliter (mL) Eppendorf tube. 7 mg of Atto633-NHS is weighed into another Eppendorf tube and dissolved in 200 μL of dimethylformamide (DMF). A volume of 300 μL of the peptide solution is added to the solution containing Atto633-NHS. The resulting solution is mixed and heated to 50° C. for 20 minutes (min). The extent of the reaction is followed with reverse-phase thin layer chromatography (TLC). A 1 μL aliquot of the reaction solution is removed and dissolved in 40 μL water and spotted on reverse phase TLC. A co-spot with Atto633 acid is included, and Atto633 is also run alone. The plate is eluted with a 2:1 solution of acetonitrile 0.1M triethylammonium acetate (TEAA). Atto633 acid and Atto633-NHS both have an R_{f} of zero, while Gly-Hyp10 has an R_{f} of 0.4. The product is purified by injecting the solution onto a C18 reverse phase column using the gradient 20%→50% acetonitrile vs. 0.1M TEAA over 16 minutes at 2.5 mL/min. The desired product is the major product, Atto633-Gly-Hyp10, eluting at 15.2 minutes. The fractions containing the desired material are collected in Eppendorf tubes and dried, yielding a blue solid. A major peak was observed on ESI mass spec: m/z calculated for C₈₇H₁₁₅N₁₄O₂₄⁺, [M]⁺=1739.8; found: 1740.6.

Preparation of Atto633-Gly-Hyp10-PFP. (FIG. 19 process (b)) Atto633-Gly-Hyp10 is suspended in 100 μL DMF in a 1.5 mL Eppendorf tube. Pyridine (20 μL) and pentafluorophenyl trifluoroacetate (PFP-TFA, 20 μL) are added to the tube. The reaction mixture is warmed to 50° C. in a heat block for 20 min. The reaction is monitored by removing 1 μL aliquots and adding to 1 mL of dilute HCl (0.4%). When the reaction is complete the aqueous solution is colorless. After 10 min the dilute HCl solution is light blue. Additional PFP-TFA (30 μL) is added. After another 100 min at 50° C. a retest of precipitation gives a colorless solution. The remaining reaction mixture is precipitated into 1 mL dilute HCl in 20 μL portions. 20 μL is added to 1 mL dilute HCl, the tube spun down, and aqueous solution discarded. The process is repeated until all of the product is precipitated. The residue is thoroughly dried. After drying, the solid is washed twice with 1 mL methyl tert-butyl ether (MTBE). The product is a dark blue powder. The product gives a major peak on electrospray ionization (ESI)-mass spectrometry (MS): m/z calculated for C₉₃H₁₁₅F₅N₁₄O₂₄²⁺, [M+H]²⁺=1906.8/2=953.4; found: 953.4.

Preparation of Atto633-Gly-Hyp10-epSS. (FIG. 19 process (c)) Atto633-Gly-Hyp10-PFP (1.6 micromoles (μmol)) is dissolved in 100 μL DMF in an Eppendorf tube. A solution of aminoethyl-SS-propionic acid (Broadpharm; 6 mg in 200 μL 0.1 M bicarbonate) is mixed with the Atto633-gly-hyp10-PFP and heated to 50° C. in a heat block for 20 min. Atto633-Gly-Hyp10-epSS is purified from the resulting reaction mixture by reverse phase HPLC using a gradient of 20%→50% acetonitrile over 16 min. Atto633-Gly-Hyp10 elutes at 15 min and Atto633-Gly-Hyp10-epSS elutes at 15.6 min. The fractions containing the product, Atto633-Gly-Hyp10-epSS, are combined and dried. The product has a major peak on ESI-MS: m/z calculated for C₉₂H₁₂₄N₁₅O₂₅S₂⁺, [M]⁺=1902.8; Found: 1902.6.

Preparation of Atto633-Gly-Hyp10-epSS-PFP. (FIG. 19 process (d)) Atto633-Gly-Hyp10-epSS is dissolved in 100 μL DMF in an Eppendorf tube. Pyridine (20 μL) and PFP-TFA (20 μL) are added and the mixture is heated to 50° C. in a heat block for 20 min. A test aliquot (1 μL) in dilute HCl gives a colorless solution and a blue precipitate. The reaction is precipitated in 20 μL aliquots in 1 mL dilute HCl, the tube spun down, and the aqueous solution discarded. The process is repeated until all of the PFP ester is precipitated. The residue is thoroughly dried under vacuum and washed with MTBE.

Preparation of dGTP-AP-epSS-Atto633. (FIG. 19 process (e)) A solution of aminopropargyl dGTP (Trilink; 1 μmol in 100 μL of 0.2 M bicarbonate) is added to 50 μL of a DMF solution comprising Atto633-gly-hyp10-epSS-PFP. The mixture is heated to 50° C. for 10 min. The product, dGTP-AP-epSS-Atto633, is purified by reverse-phase HPLC using a gradient of 20%→50% acetonitrile 16 min. The product elutes at 15.3 min. Preparative HPLC provides 0.65 μmol. The product gives a major peak on ESI-MS: m/z calculated for C₁₀₆H₁₃₉N₂₀O₃₇P₃S₂₂⁻, [M−H]²⁻, 1220.4; found: 1220.6.

While synthesis of dGTP-Atto633-Gly-Hyp10-epSS-PFP is described, a skilled practitioner will recognize that other fluorescently labeled nucleotides can be produced in a similar manner using appropriate starting materials.

### Example 12: Preparation of Dye-Labeled Nucleotides

A set of dye-labeled nucleotides designed for excitation at about 530 nm is prepared. Excitation at 530 nm may be achieved using a green laser, which may be readily available, high-powered, and stable. There are many commercially available fluorescent dyes with excitation at or near 530 nm that are inexpensive and have a variety of properties (hydrophobic, hydrophilic, positively charged, negatively charged). Synthetic routes to such dyes may be shorter and cheaper than those for longer wavelength dyes. Moreover, certain green dyes may have significantly less self-quenching than red dyes, potentially allowing for the use of higher labeling fractions (e.g., as described herein).

A viable reagent set for use in, e.g., a sequencing application consists of each of four canonical nucleotides or analogs thereof with cleavable green dyes that perform well in sequencing. An optimal set may be prepared by varying each component of a labeled nucleotide structure to obtain an array of candidate labeled nucleotides with varying properties. The resultant nucleotides are evaluated (e.g., as described below), and certain labeled nucleotides are optimized for concentration and labeling fraction (the ratio of labeled to unlabeled nucleotide in a flow).

FIG. 20 shows a variety of components that may be used in the construction of labeled nucleotides. A nucleotide can be modified with a cleavable linker moiety, a semi-rigid linker moiety such a linker moiety comprising one or more amino acids, and a fluorescent dye moiety. The nucleotides shown in FIG. 20 are propargylamino functionalized nucleotides (A, C, G, and U), but any other useful nucleotide or nucleotide analog with any other useful chemical handle can be used. Cleavable linker moieties include, for example, the structures shown as “E”, “B”, “Y”, “P”, and “Q”. Each cleavable linker moiety includes a cleavable group (e.g., as described herein). For example, cleavable linker moieties E, B, Y, P, and Q include disulfide bonds. A linker moiety (e.g., a semi-rigid linker moiety) may comprise one or more amino acid moieties, including, for example, one or more hydroxyproline moieties (e.g., as described herein). For example, a linker moiety may comprise a hydroxyproline linker (hype). The “H” linker moiety illustrated in FIG. 20 is hyp10 moiety. In some cases, a fluorescently labeled nucleotide may comprise multiple hyp10 moieties in the same or different regions of the chemical structure. For example, a linker moiety may comprise 2 or more hyp10 moieties (e.g., a hyp20 or hyp30 moiety, each of which may include 10 hydroxyproline moieties and, in some cases, another moiety such as a glycine moiety, as described herein) in sequence, which moieties may be separated by one or more other moieties or features. In some cases, a linker moiety may comprise the “D” moiety shown in FIG. 20. A linker may include multiple different portions including multiple different amino acid sequences including 2 or more amino acids (e.g., as described herein). In some cases, a fluorescently labeled nucleotide may comprise a branched or dendritic structure (e.g., as described herein) comprising multiple linker moieties (e.g., multiple sets of hydroxyproline moieties connected at different branch points to a central structure), which linker moieties may be the same or different. A fluorescently labeled nucleotide may also include one or more fluorescent dye moieties. A fluorescent dye moiety may be a structure shown in FIG. 20 as “*”, “#”, or “$” or any other useful structure. Throughout the application, these labels are used to refer to specific dye structures. However, wherever such labels are used, any other dye moiety may be substituted, including any other fluorescent dye moiety described herein. In some cases, a dye may be represented as “*”, which symbol is intended to represent any useful dye moiety or combination of dye moieties (e.g., dye pairs). Such dyes may fluoresce at or near 530 nm, or in any other useful range of the electromagnetic spectrum (e.g., as described herein). For example, red-fluorescing dyes may also be utilized. Additional examples of dye moieties are included throughout the application. There are numerous possible variations of fluorescently labeled nucleotides. Some example combinations are included in FIG. 20. For example, a fluorescently labeled nucleotide may be U*-YH (e.g., a fluorescently labeled uracil-containing nucleotide comprising a Y cleavable linker and a hyp10 moiety and a * fluorescent dye moiety), U*-YHH (e.g., a fluorescently labeled uracil-containing nucleotide comprising a Y cleavable linker and two hyp10 moieties and a * fluorescent dye moiety), U #-E (e.g., a fluorescently labeled uracil-containing nucleotide comprising an E cleavable linker and a #fluorescent dye moiety and lacking a hyp10 or similar moiety), a G*-B (e.g., a fluorescently labeled guanine-containing nucleotide comprising a B cleavable linker and a * fluorescent dye moiety and lacking a hyp10 or similar moiety), etc. Labeled nucleotides may be prepared according to synthetic routes and principles described herein.

### Example 13: Use of DPDS Increases Usable Sequencing Reads

This example illustrates that, beyond providing improvements in extension efficiency (see e.g., Example 6), the use of DPDS as a capping reagent also leads to an increased percentage of usable sequence reads for downstream analysis. Specifically, the addition of DPDS increases the percentage of high quality sequence reads, increases the synchronicity of reads, and improves homopolymer identification.

Table 1 illustrates the results of sequencing assays utilizing DPDS as a capping reagent. For instance, in the two samples where DPDS was included (e.g., samples 2 and 4), there was a lower percentage of lag phasing (e.g., 0.23 and 0.26% template lag, respectively) compared with corresponding samples (e.g., 0.31 and 0.30% template lag, respectively, for samples 1 and 3) that were not processed with DPDS. There was, however, almost no effect on template lead phasing. In addition, the samples processed with DPDS exhibited higher percentages of “flows without 3 zeros” (e.g., higher quality flows). This refers to higher quality flows that do not have more than 2 consecutive detection events that fail to record any signal; repetitive 0 signals are indicative of sequencing errors. Flows with multiple (i.e., ≥3), consecutive 0 signals are typically discarded in post-sequencing analysis, so a decrease in this type of error leads to additional usable sequence reads. For example, sample 2 exhibited almost a 10% increase in higher quality flows compared with sample 1 (i.e., 58.4% of flows in sample 2 do not include 3 zeros). Sample 4 similarly showed an improvement in the percentage of higher quality reads compared to sample 3 (e.g., ˜90% of flows in sample 4 vs ˜84% of flows in sample 3). Although there was a slight increase in template droop rates in samples 2 and 4 vs samples 1 and 3, the addition of DPDS did not greatly affect coverage or error rates.

TABLE 1
Sequencing with and without DPDS

| | Number | Number | With | Template | % Flows | Template | Template | |
|---|---|---|---|---|---|---|---|---|
| Sample | of Flows | of reads | DPDS | lag % | w/o 3 zeros | lead % | droop % | BER80 |
| 1 | 444 | 1323 | No | 0.31 | 49.1 | 0.11 | 0.41 | 0.35 |
| 2 | 444 | 1174 | Yes | 0.23 | 58.4 | 0.10 | 0.44 | 0.33 |
| 3 | 300 | 3869 | No | 0.30 | 83.9 | 0.17 | 0.63 | 0.14 |
| 4 | 300 | 3140 | Yes | 0.26 | 90.1 | 0.16 | 0.70 | 0.15 |

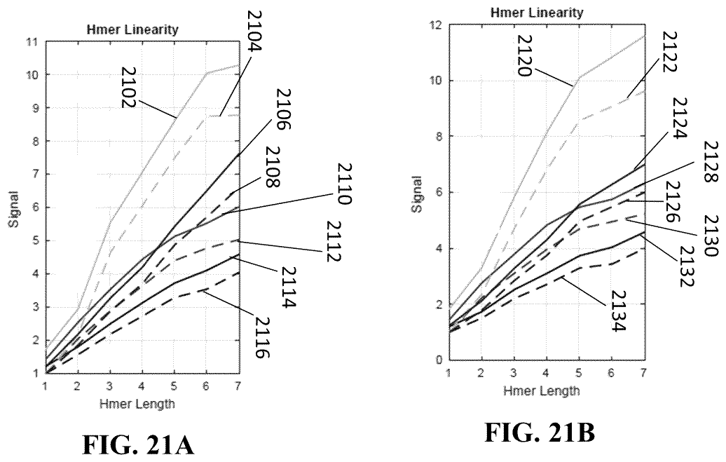

FIGS. 21A and 21B show exemplary rates of homopolymer detection (e.g., the linearity between recorded signal and homopolymer length) in sequencing assays performed with and without DPDS, respectively. A homopolymer can be of varying lengths and comprise a sequence of identical nucleotides (e.g., one nucleotide, two nucleotides, three nucleotides, four nucleotides, five nucleotides, six nucleotides, seven nucleotides, eight nucleotides, nine nucleotides, and ten nucleotides, wherein the nucleotides are all the same, i.e., all A, all T, all C, all G, etc.). Each nucleic acid template sequence evaluated in these figures is identical in sequence, except for the inclusion of a homopolymeric sequence of variable length at a predetermined locus (i.e., a sequence of 0, 1, 2, 3, 4, 5, 6, or 7 nucleotides of a same type).

Linearity measurements indicate the relationship between homopolymer length and amount of signal recorded for each nucleotide type. FIG. 21A illustrates a scenario where DPDS was used in the sequencing assay, and FIG. 21B represents data where DPDS was not included in the sequencing assay. For each nucleotide type (A, T, C, and G), the solid line in the “Hmer linearity” panels represents the amount of in phase signal and the dashed line represents the amount of out of phase signal for each length of homopolymer. The numbers on the right vertical axis indicate the difference between the amount of signal that is in and out of phase for each homopolymer type (e.g., for homopolymers of each nucleotide type). For example, in FIG. 21A, the difference between the in and out of phase reads (e.g., lines 2102 and 2104, respectively) for homopolymers of base A is 1.38, for base C homopolymers it is 0.95 (see lines 2106 and 2108), for base T homopolymers it is 0.82 (see lines 2110 and 2112), and for base G homopolymers it is 0.63 (see lines 2114 and 2116). In FIG. 21B, where the sequencing is performed without the addition of DPDS, the difference between the amount of signal recorded from in and out of phase reads is 1.51 for homopolymers of base A (e.g., lines 2120 and 2122), 0.91 for homopolymers of base C (see lines 2124 and 2126), 0.86 for homopolymers of base T (2128 and 2130), and 0.62 for homopolymers of base G (see lines 2132 and 2134). These data demonstrate that there is an increase of in phase sequencing reads, specifically for A homopolymer sequences, when DPDS is used as a capping reagent during sequencing.

### Example 14: Homopolymer Extension with Variable Length Linkers

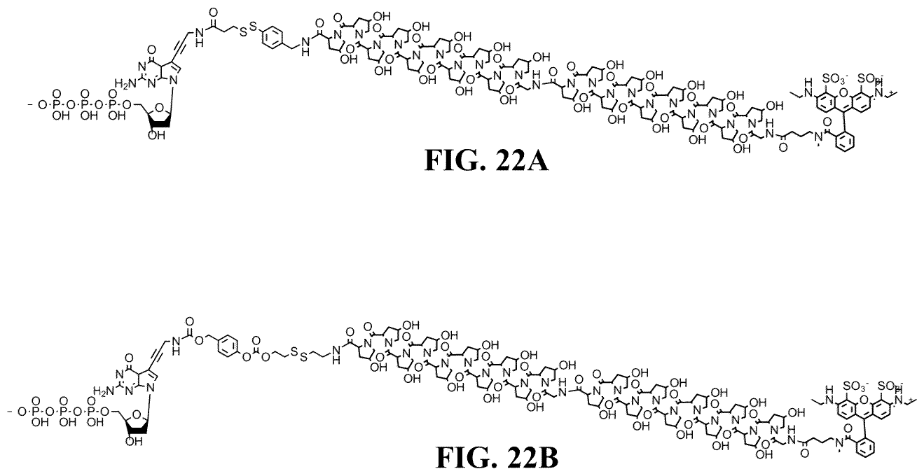

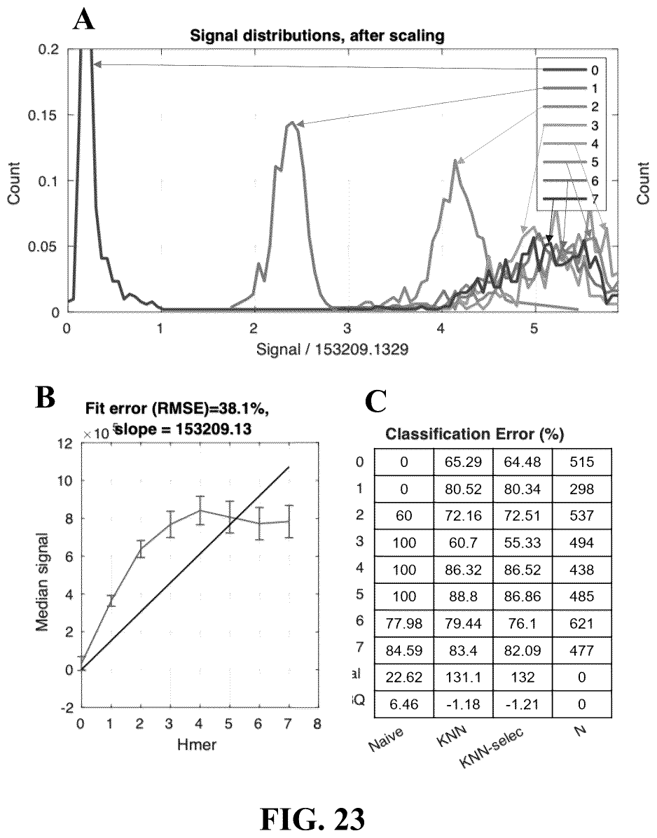

This example covers extension over homopolymeric regions using substrates coupled to varying length linkers. While linker rigidity (e.g., that conferred by some oligopeptide moieties) can be important for spacing dyes to diminish quenching, some substrate conformational flexibility is often requisite for polymerase activity. These characteristics can be balanced by varying the lengths of cleavable, flexible and rigid oligopeptide portions of a linker. In this example, two different propargyl amine functionalized guanosine nucleotides connected by different length cleavable moieties to (Hyp10)₂-dye units (FIGS. 22A-22B) were compared in homopolymer extension assays performed by extending a primer over templates containing 0- to 7-mer cytidine regions followed by an identifying sequence.

FIG. 23 provides extension results for the substrate of FIG. 22A, comprising a linker with a 9 atom spacing between the propargyl amine functionalized guanosine and the (Hyp10)₂ oligopeptide moiety, over variable length homopolymeric cytidine regions. Corrected fluorescence intensities of the oligoguanidine-containing extension products are provided in FIG. 23 panel A, showing clean and narrow bands for lower guanosine numbers. FIG. 23 panel B provides the data from FIG. 23 panel A plotted as corrected fluorescence intensities as a function of oligocytidine template length and shows that the substrate of FIG. 22A can cleanly distinguish between 0- through 3-mer length regions. FIG. 23 panel C provides classification errors for homopolymer length identifications based on the fluorescence data of FIG. 23 panels A and B.

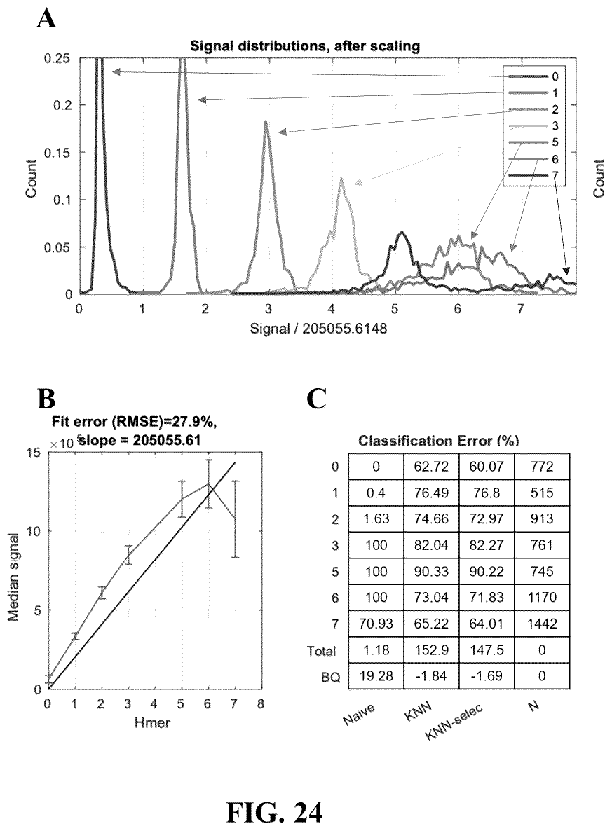

FIG. 24 provides homopolymer extension results for the substrate shown in FIG. 22B, comprising a longer linker with a 15 atom spacing between the propargyl amine functionalized guanosine and the (Hyp10)₂ oligopeptide moiety. In FIG. 24 panel A, narrow, distinct bands are readily distinguishable for each of the 0- through 3- and 5- through 7-mer homopolymer regions queried with the FIG. 22B substrate. This is reflected in the fluorescence intensity plot in FIG. 24 panel B, which is linear over the 0- through 6-mer region, showing that the substrate can cleanly distinguish homopolymer regions of up to six nucleotides in length. FIG. 24 panel C provides classification errors for the various homopolymer length assignments.

### Example 15: Homopolymer Extension with Variable Length Linkers

This example covers the effect of varying nucleobases in nucleic acid synthesis with labeled substrates. While the foregoing examples explore how linker-structure affects nucleotide extension efficiency, labeled nucleotide incorporation can also depend on nucleobase type. Two nucleotides (e.g., ATP and GTP) sharing the same linker may exhibit markedly different incorporation rates and homopolymer extension efficiencies, and therefore, an optimized reagent set may provide different linkers for different nucleotides.

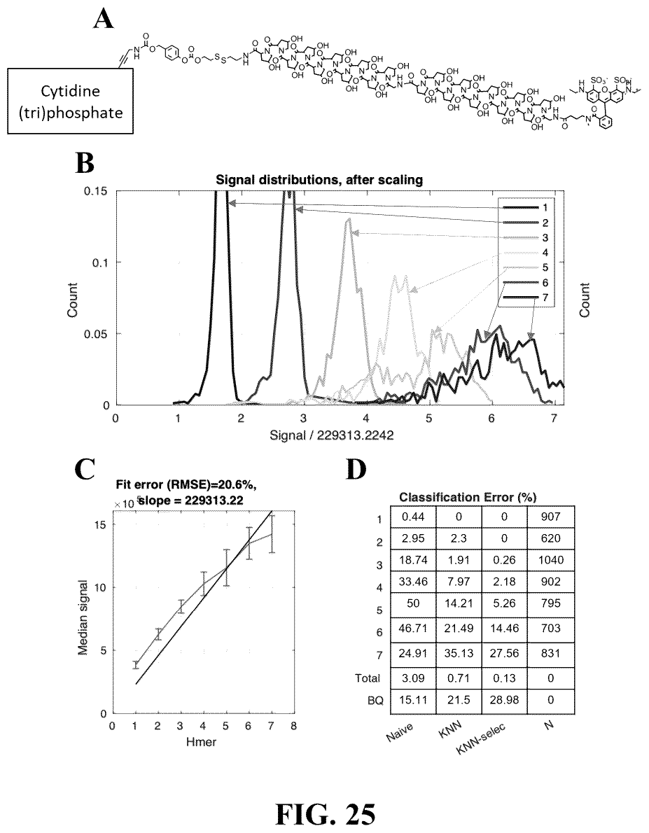

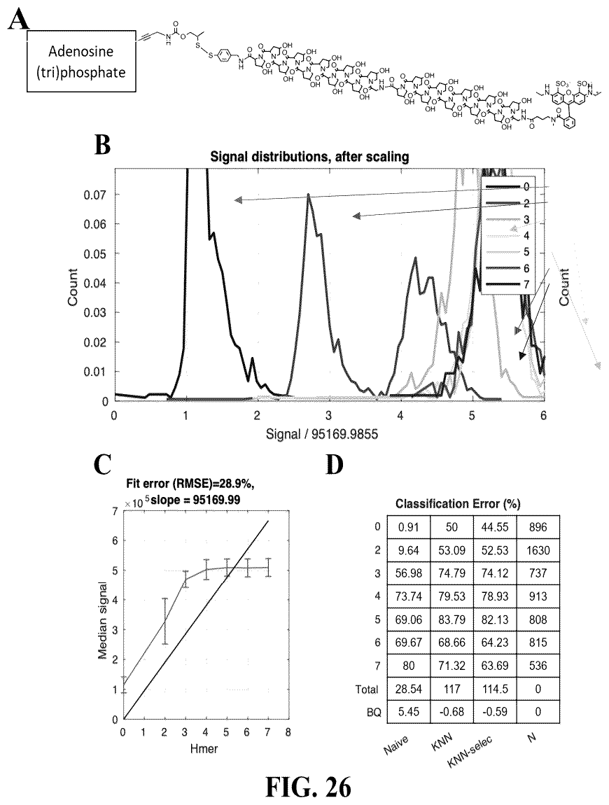

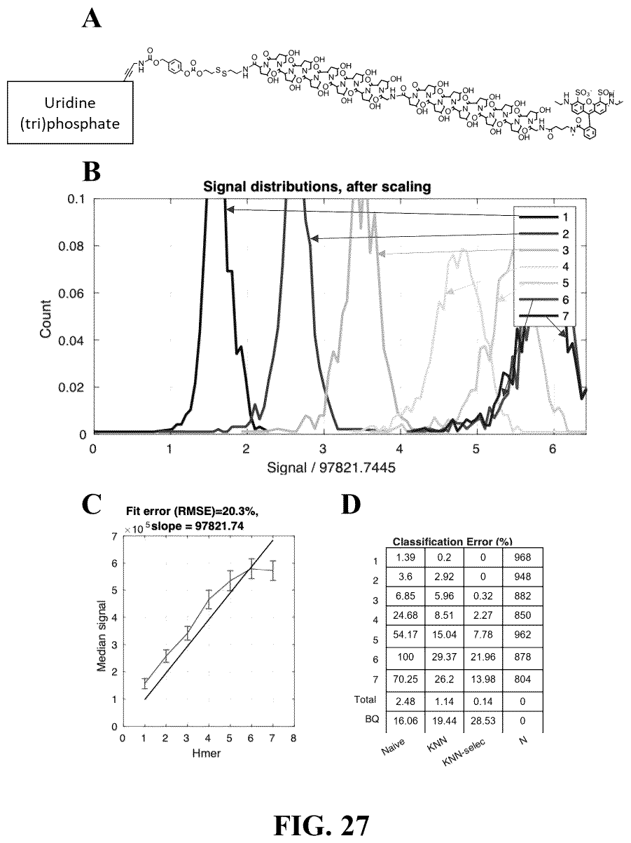

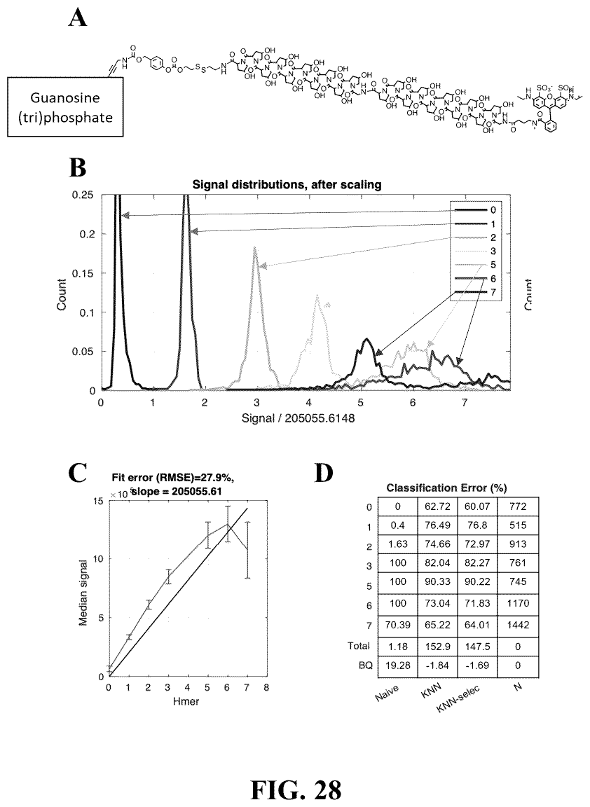

To explore these dependencies, homopolymer extension assays were performed with CTP, UTP, and GTP sharing a common linker (panel A of FIGS. 25, 27, and 28) and with ATP comprising a distinct linker with a modified cleavable region (FIG. 26 panel A). Panel B of FIGS. 25-28 provides corrected fluorescence intensities of the for extension over homopolymeric regions of varying lengths. Panel C of FIGS. 25-28 provides corrected fluorescence intensities as a function of homopolymer template length, with error bars summarizing inter-assay variation. Panel D of FIGS. 25-28 provides classification errors.

As can be seen from Panels B and C of FIGS. 25-28, the cleavable carbonate linker of FIGS. 25, 27, and 28 provided greater linearity between template homopolymeric region length and fluorescence intensity, enabling homopolymer length identification of up to six units. Among the three nucleotide substrates comprising the carbonate linker, GTP provided the narrowest bands for template lengths of fewer than 4 units, while CTP and UTP performed better for longer homopolymer templates.

### Example 16: Cleavable Linker Synthesis, Cleavage, and Post-Cleavage Decomposition

This example outlines the synthesis, cleavage, and post-cleavage reaction for a labeled nucleotide substrate. A synthesis scheme for the labeled dUTP substrate is described below. FIG. 30 provides a cleavage and post-cleavage decomposition mechanism for the labeled dUTP substrate.

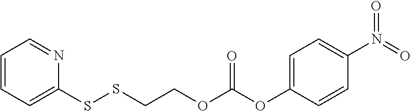 [SMILES: O=C(OCCSSc1ccccn1)Oc1ccc([N+](=O)[O-])cc1]

(NPEC, shown above) conversion to PN40338: p-(TBS-oxymethyl)phenol (45 mg, 0.19 mmol) was dissolved in DMF, to this solution a solution of NPEC (67 mg, 0.19 mmol) in DMF and triethylamine (96 mg, 0.95 mmol) were added. The mixture was left at room temperature for 5 hours, after which the solvent was evaporated. The resulting residue was purified by flash chromatography (hexanes:ethyl acetate 2:1) to yield PN40338 (40 mg, 47% yield) as a colorless oil where PN40338 comprises:

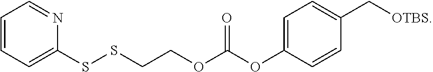 [SMILES: CC(C)(C)[Si](C)(C)OCc1ccc(OC(=O)OCCSSc2ccccn2)cc1]

PN40338 conversion to PN40374: 1 mL of 1.25M HCl in ethanol was added to a flask containing PN40338 (40 mg, 89 umol). Reaction completion was confirmed by TLC, and the solvent was evaporated to yield PN40374 (30 mg, 89 umol) as a colorless oil, where PN40374 comprises:

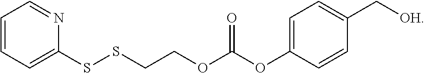 [SMILES: O=C(OCCSSc1ccccn1)Oc1ccc(CO)cc1]

PN40374 conversion to PN40375: PN40374 (30 mg, 89 umol) was dissolved in DCM, to this solution, p-Nitrobenzenyl chloroformate (30 mg, 0.15 mmol) and triethylamine (36 mg, 0.36 mmol) were added. The reaction mixture was spotted onto a prep TLC plate (20×20 cm, 2000 micron), which was eluted in a mixture of 2:1 hexanes:ethyl acetate. The product spot was removed from the plate, and the silica gel was washed with ethyl acetate. This solvent was evaporated to yield PN40375 (20 mg, 50 umol), comprising:

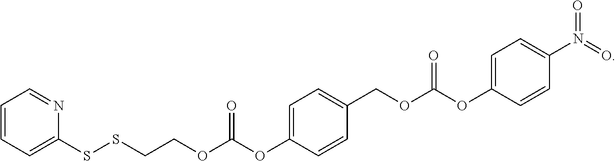 [SMILES: O=C(OCCSSc1ccccn1)Oc1ccc(COC(=O)Oc2ccc([N+](=O)[O-])cc2)cc1]

A preferable workup for this reaction would be flash chromatography, particularly for a larger reaction.

PN40375 conversion to PN40377: a portion of the PN40375 obtained from the previous reaction was dissolved in formamide. Some of the material was insoluble in formamide, so the solution was decanted and the remaining solid was dissolved in DMF. A solution of dUTP-AP (8 umol) was dried down and suspended in formamide. This solution was combined with both solutions of PN40375 as well as 40 uL DIEA. The mixture was left at room temperature overnight, and then purified by HPLC to produce PN40377 comprising:

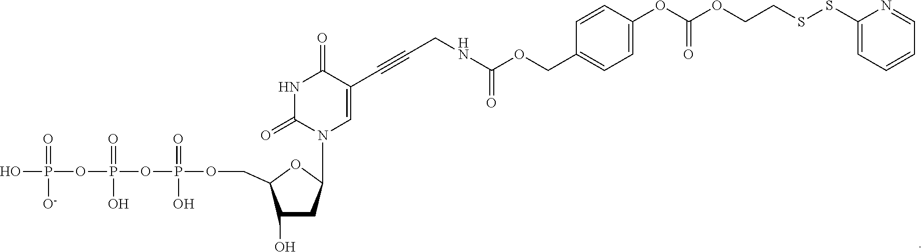 [SMILES: O=C(NCC#Cc1cn([C@H]2C[C@@H](O)[C@@H](COP(=O)(O)OP(=O)(O)OP(=O)([O-])O)O2)c(=O)[nH]c1=O)OCc1ccc(OC(=O)OCCSSc2ccccn2)cc1]

PN40377 conversion to PN40379: PN40377 (0.5 umol) was dissolved in 300 uL water. Acetic acid (1 uL) and cysteamine (11 umol) were added. The reaction mixture was kept at room temperature for two hours, then purified by HPLC. 0.2 umol PN40379 (dUTP-Q) were recovered, where PN40379 comprises:

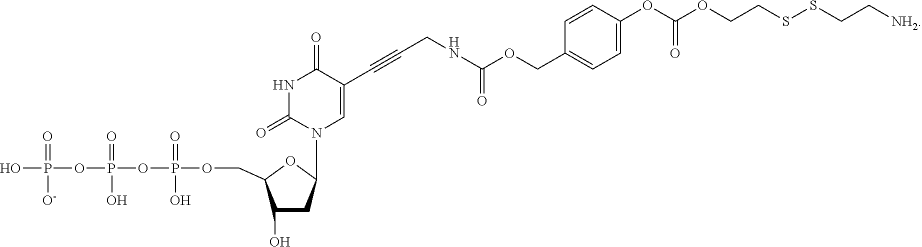 [SMILES: NCCSSCCOC(=O)Oc1ccc(COC(=O)NCC#Cc2cn([C@H]3C[C@@H](O)[C@@H](COP(=O)(O)OP(=O)(O)OP(=O)([O-])O)O3)c(=O)[nH]c2=O)cc1]

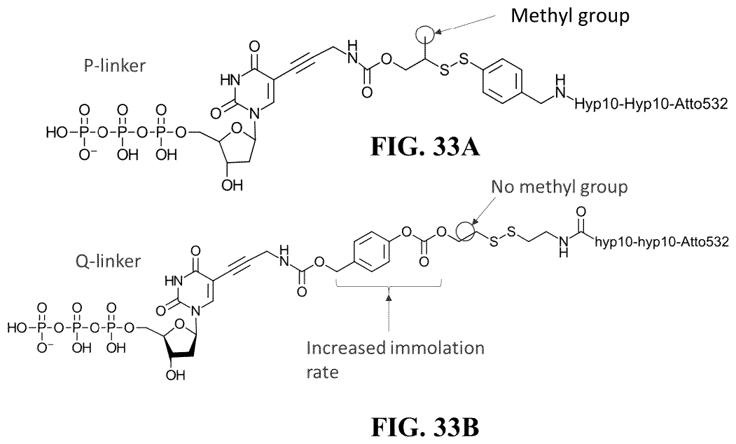

PN40379 conversion to the labeled dUTP product: 0.2 umol PN40379 were dissolved in water and combined with a solution of Atto532-hyp10-hyp10-PFP (0.6 umol) in DMF. A total of 150 uL of 1M bicarbonate were added in portions over several hours. The reaction mixture was purified by HPLC, yielding 0.06 umol of U*QHH (e.g., structure 3001 in FIG. 30). A further example of a Q-linker is provided in FIG. 20 and FIG. 33B.

FIG. 30 provides a reaction scheme for a cleavage and post-cleavage reaction of the labeled dUTP, synthesized as described above. Cleavage of the labeled dUTP substrate 3001 may be achieved with a reducing agent, such as THP, resulting in cleavage 3010 of the disulfide to form thiolate scars on the dUTP substrate and the (Hyp10)₂-dye moiety (see structure 3002). The dUTP thiolate scar can spontaneously decompose 3011 to liberate a thiirane and a molecule of CO₂. The resulting phenolate scarred dUTP 3003 can further decompose 3012 to liberate 4-methylenecyclodien-1-one and an additional molecule of CO₂, yielding dUTP with a propargyl amine scar 3004.

### Example 17: Cleavable Linker Synthesis, Cleavage, Post-Cleavage Decomposition, and Assays

As described herein, a labeling reagent may include a cleavable moiety comprising a cleavable group. The inclusion of a cleavable moiety in a labeling reagent may facilitate separation of the labeling reagent or a portion thereof from a substrate to which it is coupled.

In this example, three different propargyl amine functionalized uridine nucleotides connected by different cleavable moieties to (Hyp10)₂-dye units were compared in homopolymer extension assays performed by extending a primer over templates containing 0- to 7-mer adenosine regions followed by an identifying sequence.

To compare the performance of three labeled uracil-containing nucleotides, sequencing assays were performed using U*-YHH (e.g., a uracil-containing nucleotide labeled with a labeling agent comprising a * dye, a Y cleavable linker, and two hyp10 moieties), U*-PHH (e.g., a uracil-containing nucleotide labeled with a labeling agent comprising a * dye, a P cleavable linker, and two hyp10 moieties; shown in FIG. 33A), and U*-QHH (e.g., a uracil-containing nucleotide labeled with a labeling agent comprising a * dye, a Q cleavable linker, and two hyp10 moieties; shown in FIG. 33B). The U*-PHH linker self-immolates, but incorporation is slow as evidenced by poor multiple incorporations, the results of which were then corroborated by solution phase assays. The U*-QHH linker self-immolates and is rapidly incorporated; and the homopolymer assay results are as good as or better than that of the Y-linker.

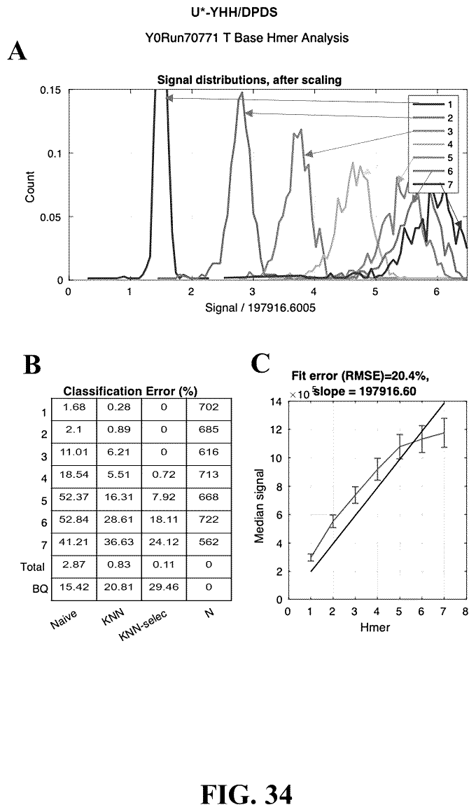

FIG. 34 provides extension results for the substrate U*-YHH/DPDS over variable length homopolymeric adenosine regions. The U*-YHH linker leaves a thiol that is subsequently capped with DPDS. Panel A provides corrected fluorescence intensities of the oligodeoxyuridine phosphate-containing extension products. Panel B provides the data from Panel A plotted as corrected fluorescence intensities as a function of adenosine template length. Panel C provides classification errors for homopolymer length identifications based on the fluorescence data of Panels A and B.

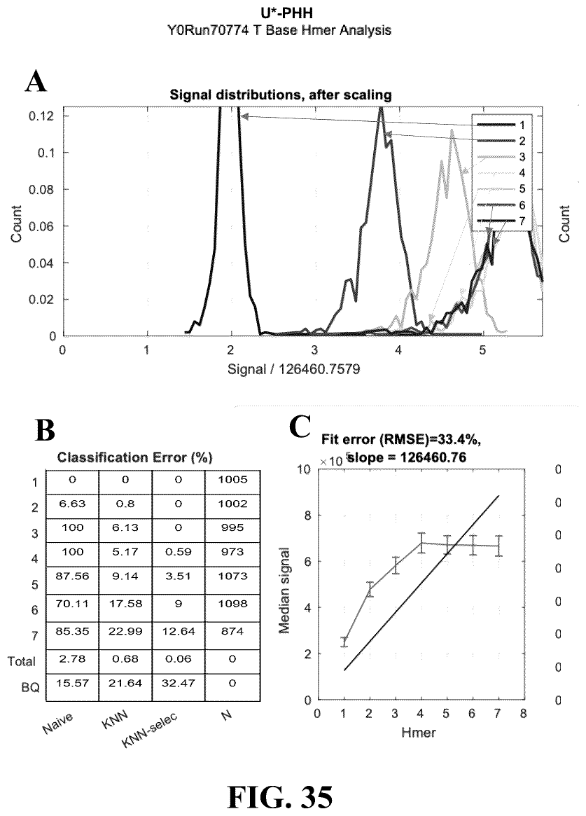

FIG. 35 provides extension results for the substrate of FIG. 33A, comprising a linker with a 10 atom spacing between the propargyl amine functionalized uridine and the (Hyp10)₂ oligopeptide moiety, over variable length homopolymeric adenosine regions. Corrected fluorescence intensities of the oligoguanidine-containing extension products are provided in FIG. 35 Panel A. FIG. 35 Panel B provides the data from FIG. 35 Panel A plotted as corrected fluorescence intensities as a function of adenosine template length and shows that the substrate of FIG. 33A can cleanly distinguish between 0- through 3-mer length regions. FIG. 35 Panel C provides classification errors for homopolymer length identifications based on the fluorescence data of FIG. 35 Panels A and B.

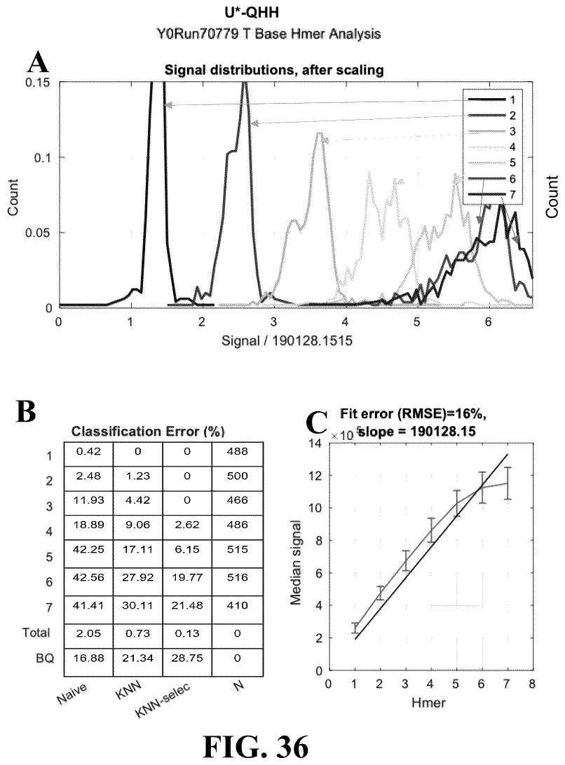

FIG. 36 provides homopolymer extension results for the substrate shown in FIG. 33B, comprising a longer linker with a 15 atom spacing between the propargyl amine functionalized guanosine and the (Hyp10)₂ oligopeptide moiety. In FIG. 36 Panel A, narrow, distinct bands are readily distinguishable for each of the 0- through 5-mer homopolymer regions queried with the FIG. 33B substrate. This is reflected in the fluorescence intensity plot in FIG. 36 Panel B, which is linear over the 0- through 5-mer region, showing that the substrate can cleanly distinguish homopolymer regions of up to five nucleotides in length. FIG. 36 Panel C provides classification errors for the various homopolymer length assignments.

### Example 18: Sequencing Assays with Cleavable Linkers

In this example, a sequencing assay was performed by extending a primer against a template nucleic acid in the presence of alternating flows of single fluorescently labeled nucleotides provided as cycles of U, C, A, and then G.

In between each flow, the fluorescent labels were detected and cleaved. Each labeled nucleotide flow was then followed by a “dark tap” flow consisting of 100% unlabeled nucleotides to maintain phasing. Capping reagent DPDS was not provided during this test. The labeled nucleotides used were A*-PHH, C*-PHH, G*-PHH, and U*-QHH.

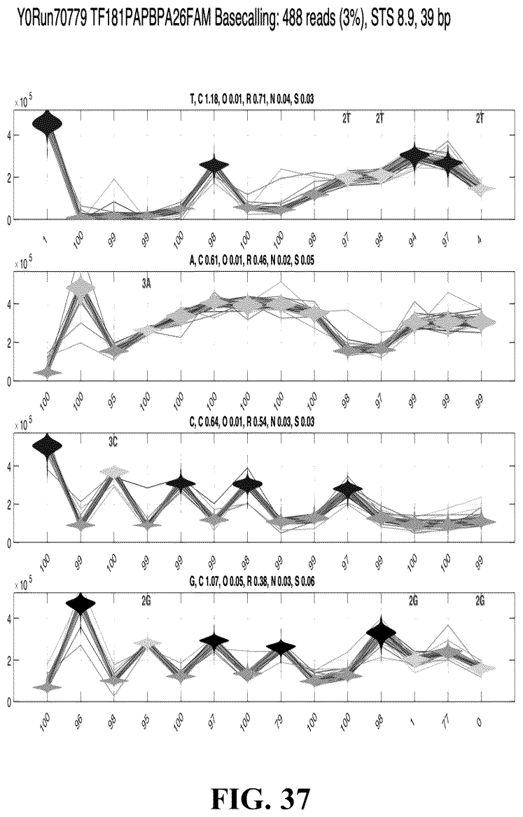

The results of the assay are provided in FIG. 37. In FIG. 37, the y-axes provide fluorescence intensity following labeled nucleotide incorporation, and the four plots (from top to bottom) separately provide the results of uridine, adenosine, cytidine, and guanosine incorporation steps, with individual cycles progressing from left to right.

FIG. 37 summarizes the sequencing results of an assay utilizing flows with 100% labeled U, 100% labeled C, 100% labeled A, 100% labeled G, and no capping reagent. It can be seen that labeled nucleotide incorporation, and cleavage of the dye, does not noticeably inhibit subsequent nucleotide incorporations.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

1.-135. (canceled)

136. A method for sequencing, comprising:
(i) incorporating a nucleotide into a growing nucleic acid strand hybridized to a nucleic acid molecule, wherein said nucleotide is coupled to a dye via a cleavable linker;
(ii) detecting a signal or change thereof from said dye;
(iii) cleaving said cleavable linker to generate a reactive moiety on said nucleotide;
(iv) contacting a capping reagent to said reactive moiety to generate a capped moiety; and
(v) incorporating an additional nucleotide adjacent to said nucleotide in said growing nucleic acid strand.

137. The method of claim 136, wherein said nucleotide is a terminated nucleotide.

138. The method of claim 136, wherein said nucleotide is a non-terminated nucleotide.

139. The method of claim 136, wherein said incorporating in (i) comprises contacting said growing nucleic acid strand with a reaction mixture comprising a plurality of nucleotides of a same canonical base type.

140. The method of claim 139, wherein said contacting incorporates at least two nucleotides of said plurality of nucleotides into said growing nucleic acid strand, wherein said at least two nucleotides are coupled to dyes via cleavable linkers, and wherein said at least two nucleotides comprises said nucleotide.

141. The method of claim 139, wherein said plurality of nucleotides comprises a subset of labeled nucleotides and a subset of unlabeled nucleotides.

142. The method of claim 136, wherein said cleaving in (iii) comprises cleaving a disulfide group of said cleavable linker.

143. The method of claim 136, wherein said cleaving in (iii) comprises reduction of said cleavable linker via one or more reducing agents selected from the group consisting of tetrahydropyran, β-mercaptoethanol (β-ME), dithiothreitol (DTT), tris(2-carboxyethyl)phosphine (TCEP), Ellman's reagent, hydroxylamine, glutathione, and cyanoborohydride.

144. The method of claim 136, wherein said reactive moiety comprises a thiol.

145. The method of claim 136, wherein said capping reagent comprises a reversible capping reagent.

146. The method of claim 145, wherein said reversible capping reagent is selected from the group consisting of 4-4′-dipyridyl disulfide, 2,2′-dithiobis(5-nitropyridine), 6,6′-dithiodinicotinic acid, and dipyridyl disulfide (DPDS).

147. The method of claim 136, wherein said capping reagent comprises an irreversible capping reagent.

148. The method of claim 147, wherein said irreversible capping reagent is selected from the group consisting of a haloacetamide, a maleimide, and a propioloate.

149. A method for sequencing, comprising:
(a) incorporating a nucleotide into a growing nucleic acid strand hybridized to a nucleic acid molecule, wherein said nucleotide is coupled to a dye via a cleavable linker;
(b) detecting a signal or change thereof from said dye;
(c) cleaving said cleavable linker to generate a reactive moiety on said nucleotide;
(d) contacting said growing nucleic acid strand with a mixture comprising a capping reagent and an additional nucleotide, wherein said capping reagent is configured to couple to said reactive moiety to generate a capped moiety.

150. The method of claim 149, wherein said mixture comprises a plurality of nucleotides of a same canonical nucleotide type.

151. The method of claim 150, wherein said plurality of nucleotides comprises a subset of labeled nucleotides and a subset of unlabeled nucleotides.

152. The method of claim 149, wherein said mixture comprises a plurality of nucleotides of at least two canonical nucleotide types, and wherein the at least two canonical nucleotide types are coupled to different detectable moieties comprising different fluorescent dyes.

153. The method of claim 149, wherein said mixture comprises a plurality of nucleotides of a same canonical base type, wherein said mixture does not comprise a labeled nucleotide, wherein said plurality of nucleotides comprises said additional nucleotide.

154. The method of claim 149, wherein said nucleotide and said additional nucleotide comprise a same type of canonical nucleobase.

155. The method of claim 149, wherein said additional nucleotide comprises a detectable moiety, and further comprising (e) detecting an additional signal from said detectable moiety of said additional nucleotide, and (f) using said signal from (b) and said additional signal from (e) to determine a sequence of said nucleic acid molecule.
